(19) United States
(12) Patent Application Publication
(10) Pub. No.: US 2024/0034735 A1
(43) Pub. Date: Feb. 1, 2024

(54) NOVEL COMPOUNDS

(71) Applicant: AC Immune SA, Lausanne (CH)

(72) Inventor: Jérôme MOLETTE, Lausanne (CH)

(73) Assignee: AC Immune SA, Lausanne (CH)

(21) Appl. No.: 18/222,115

(22) Filed: Jul. 14, 2023

(30) Foreign Application Priority Data
Jul. 14, 2022 (EP) 22 185 021.7

(51) Int. Cl. C07D 487/04; A61P 25/28; A61P 25/16

(52) U.S. Cl. CPC C07D 487/04; A61P 25/28; A61P 25/16

(57) ABSTRACT

The present invention relates to novel compounds for the treatment, alleviation or prevention of a group of diseases, disorders and abnormalities which are responsive to the modulation or inhibition of the activation of a component of the NLRP3 inflammasome pathway. In particular, the component of the inflammasome pathway is a NOD-like receptor (NLR) family, pyrin domain-containing protein 3 (NLRP3) inflammasome. More particularly, the compounds of the present invention have the capability to modulate the NLRP3 inflammasome pathway. Further, the compounds of the present invention are suitable for the treatment, alleviation or prevention of a group of diseases, disorders and abnormalities which are responsive to the modulation, in particular decrease, of IL-1 beta and/or IL-18 levels.

### FIELD OF THE INVENTION

The present invention relates to novel compounds that are useful for the treatment, alleviation or prevention of a disease, disorder or abnormality which is responsive to the modulation, in particular inhibition of the activation, of a component of the NLRP3 inflammasome pathway. In particular, the component of the inflammasome pathway is NOD-like receptor (NLR) family, pyrin domain-containing protein 3 (NLRP3) inflammasome. More particularly, the compounds of the present invention have the capability to modulate, e.g., inhibit the activation of, the NLRP3 inflammasome pathway. Further, the compounds of the present invention have the capability to modulate, in particular decrease, IL-1 beta and/or IL-18 levels. The present invention relates to novel compounds for the treatment, alleviation or prevention of a disease, disorder or abnormality which is responsive to the inhibition of the activation of the NLRP3 inflammasome pathway. The present invention relates to novel compounds for the treatment, alleviation or prevention of a disease, disorder or abnormality which is responsive to the modulation of IL-1 beta and/or IL-18 levels. The present invention relates to pharmaceutical compositions comprising said compounds, methods of using said compounds in the treatment of various diseases, disorders or abnormalities which is responsive to the above-mentioned modulation, medicaments containing them and their uses thereof.

### BACKGROUND OF THE INVENTION

Inflammasome protein complexes are the key components of inflammatory signalling. These complexes assemble in response to various danger signals such as molecules from infectious agents (pathogen-associated molecular patterns, PAMPs) as well as altered host molecules, products of sterile tissue damage and environmental factors (danger associated molecular patterns, DAMPs). The inflammasome family consists of NALP1-14, IPAF, and NAIP 1-6, with each family member providing specificity towards different PAMPs/DAMPs including nucleic acids, bacterial proteins, metabolites, protein aggregates and the activity of toxins (Sharma, D. & Kanneganti, T. D. The cell biology of inflammasomes: mechanisms of inflammasome activation and regulation. J. Cell Biol. 213, 617-629 (2016)). Inflammasomes are typically composed of a sensor (a cytosolic pattern-recognition receptor, PRR) and an adaptor protein called apoptosis associated speck-like protein containing a caspase-recruitment domain (CARD) (ASC), and an effector such as the protease caspase-1 (Broz, P.; Dixit, V. M. Inflammasomes: Mechanism of Assembly, Regulation and Signalling. Nat. Rev. Immunol. 2016, 16, 407-420).

NLRP3 (NOD-like receptor (NLR) family, pyrin domain-containing protein 3) inflammasome is one of the best-described family members. It is a tripartite protein of the NLR family and contains an amino-terminal PYRIN (PYD) domain, a nucleotide-binding NACHT domain and a carboxy-terminal leucine-rich repeat (LRR) domain. In response to various agents including aggregated proteins, crystals and altered cellular ion homeostasis, the NLRP3 sensor molecule assembles into a multi-molecular complex with apoptosis-associated speck-like protein containing a caspase activation and recruitment domain (ASC aka PYCARD) adaptor protein. ASC protein polymerization into a large complex (ASC speck) leads to activation of caspase-1 effector protein and subsequent cleavage of pro-IL-1 beta (β) and pro-IL18 into their active secreted forms and mediates pyroptosis (Heneka et al., 2018 Nat Rev Neurosci). IL-1 beta (β) acts through IL-1 beta (β) receptors, induces secondary pro-inflammatory signals including IL-6 and TNF alpha secretion, and attracts and activates cells of adaptive immune system at the sites of infection. NLRP3/ASC complexes seems to be released into the extracellular environment where they can propagate inflammation.

Multiple genetic and pharmacological evidence highlight the importance of NLRP3 inflammasome in human disease. NLRP3 gain-of-function mutations lead to the inherited cryopyrin-associated periodic syndromes (CAPS) including Muckle-Wells syndrome (MWS), familial cold auto-inflammatory syndrome (FCAS) and neonatal-onset multisystem inflammatory disease (NOMID).

Accumulation of tissue damage products associated with ageing results in activation of NLRP3 inflammasome in multiple diseases including metabolic disorders, Alzheimer's disease, Parkinson's disease, amyotrophic lateral sclerosis, atherosclerosis, obesity, lung diseases, liver diseases and gout.

Vast experimental evidence from animal models points out the detrimental role of excessive NLRP3 activation in a wide spectrum of diseases. NLRP3-inflammasome genetic or pharmacological downregulation showed protection in models of Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 and type 2 diabetes, rheumatoid arthritis, myelodysplastic syndrome, among others (Heneka et al., Nat. Rev. Neurosci. 2018 October; 19(10):610-621; Mangan et al., Nat. Rev. Drug Discov. 2018 August; 17(8):588-606).

For the reasons described above modulation of NLRP3 inflammasome pathway activity represents a promising therapeutic approach.

Current treatments for NLRP3-related diseases include biologics targeting IL-1. These are the recombinant IL-1 receptor antagonist anakinra, the neutralizing IL-1 beta (β) antibody canakinumab and the soluble decoy IL-1 receptor rilonacept. However, their activity is limited to downstream effectors of inflammasome and their bioavailability for central nervous system (CNS) applications is limited.

Several small molecules have been shown to inhibit the NLRP3 inflammasome pathway (Baldwin, A. G., Brough, D. & Freeman, S. Inhibiting the NLRP3 inflammasome pathway: a chemical perspective. J. Med. Chem. 59, 1691-1710 (2016); reviewed in Mangan et al., Nat Rev Drug Discov. 2018 August; 17(8):588-606). These include various chemical classes such as sulfonylurea-based compounds (glyburide, CP-456,773 (aka CRID3 and MCC950) and its derivatives); fenamate classes of non-steroidal anti-inflammatory drugs; hydroxysulfonamide analogue JC-171; novel boron compound series; benzimidazole-containing structure Fc11a-2; polyketide spirodalesol; acrylate and acrylamide derivatives; 3,4-methylenedioxy-β-nitrostyrene; β-sulfonyl nitrile molecule OLT1177; CY-09; BOT-4-one; and Michael acceptors. Most of these compounds have a promiscuous mode of action and limited potency.

WO2016131098, WO2017/140778 and WO2018215818 refer to sulfonylurea and related compounds and their use in treating or identifying a disease or condition responsive to inhibition of NLRP3 or inhibition of the activation of NLRP3 or related components of the inflammatory process.

WO2019008025, WO2019008029, WO2019034686, WO2019034688, WO2019034690, WO2019034692, WO2019034693, WO2019034696, WO2019034697, WO2019068772, WO2019092170, WO2019092171 and WO2019092172 refer to novel compounds (e.g. sulfonylureas, sulfonylthioureas, sulfoximine ureas and sulfoximine thioureas), useful in the treatment and prevention of medical disorders and diseases, most especially by NLRP3 inhibition.

WO2017184604, WO2017184623, WO2017184624, WO2019023145, WO2019023147 and WO2019079119 refer to chemical entities that are useful for treating a condition, disease, or disorder in which a decrease or increase in NLRP3 activity contributes to the pathology and/or symptoms and/or progression of the condition, disease, or disorder in a subject.

WO2019211463, WO2020021447, and WO2021043966, WO2021239885, WO2021219784, WO2021214284, WO2021209552, WO2021209539 disclose compounds for inhibiting NLRP3 and/or NLRP3 inflammasome pathway.

WO2018136890 refers to sulfonylurea and sulfonyl thiourea compounds and their use in treating a disease or condition responsive to modulation of cytokines such as IL-1 beta ((3) and IL-18, modulation of NLRP3 or inhibition of the activation of NLRP3 or related components of the inflammatory process.

WO2018225018 and WO2019043610 refer to NLRP3 modulators as well as to the use of the novel inhibitor compounds in the treatment of diseases or conditions as well as treatment of disease states mediated by NLRP3 as well as treatment of diseases or conditions in which interleukin 1 beta (β) activity and interleukin-18 (IL-18) are implicated.

WO2018015445 refers to sulfonylurea compounds which possess inflammasome inhibitory activity and are accordingly useful in methods of treatment of the human or animal body.

WO2020018975 discloses sulfonimidamide derivatives defined as inhibitors of interleukin-1 activity and NLRP3 modulators in connection with inflammatory processes.

WO9832733 refers to aryl and heteroaryl substituted sulfonyl ureas that are inhibitors of interleukin-1 alpha (α) and interleukin-1 beta (β) processing and release.

WO2020018970 discloses sulfonylureas defined as inhibitors of interleukin-1 activity.

WO2020/234715 discloses pyridazine-3-yl phenol compounds defined as inhibitors of NOD-like receptor protein 3 (NLRP3) inflammasome activity.

WO2021/193897 refers to substituted pyridazine compounds that are described as having suppressive action on NLRP3 inflammasome activity.

The crosstalk between the NLRP3 inflammasome pathway and Tau pathology has been recently deciphered. Ising et al. (Nature 2019 November; 575(7784):669-673) investigated the important role of microglia and NLRP3 inflammasome pathway activation in the pathogenesis of tauopathies in the Tau22 mouse model of FrontoTemporal Dementia (FTD). Genetic ablation of components of the NLRP3 inflammasome pathway in Tau22 mice reduced Tau aggregation/phosphorylation as well as improved cognition. Stancu et al. (Acta Neuropathol. 2019; 137(4): 599-617) investigated the role of inflammasome activation in prion-like or templated seeding of Tau pathology. Significant inhibition of exogenously seeded Tau pathology was found in ASC deficient—PS19 Tau transgenic mice. Furthermore, it was demonstrated that chronic intracerebral administration of the NLRP3 inhibitor, MCC950, inhibits exogenously seeded Tau pathology. Finally, ASC deficiency also decreased non-exogenously seeded Tau pathology in PS19 mice.

There is a need to identify and develop specific NLRP3 inflammasome pathway inhibitors and/or modulators of interleukin activity with beneficial pharmacological and/or physiological and/or physicochemical properties.

The present invention provides compound of formula (I′), (I), (Ia), (Ib), (Ic), (Id) or (Ie) which have surprisingly been found to be capable of modulating a component of the NLRP3 inflammasome pathway, in particular inhibiting the activation, of a component of the NLRP3 inflammasome pathway, such as NLRP3 inflammasome. Thus, such compounds are beneficial in the treatment of a disease, disorder, or abnormality which is responsive to the modulation of a component of the NLRP3 inflammasome pathway and/or which is responsive to the modulation of IL-1 beta and/or IL-18 levels that commonly lead to pathological inflammation.

### SUMMARY OF THE INVENTION

Accordingly, the present invention provides compounds that can be employed in the treatment, alleviation or prevention of a disease, disorder or an abnormality which is responsive to the modulation, in particular inhibition, of a component of the NLRP3 inflammasome pathway, or which is responsive to the modulation, in particular decrease, of IL-1 beta and/or IL-18 levels.

Various embodiments of the invention are described herein.

Within a certain aspect, provided herein is a compound of formula (I)

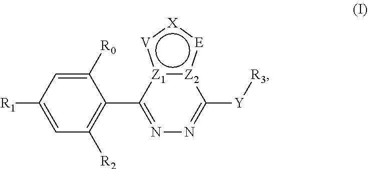

or a stereoisomer, a racemic mixture, a tautomer, a polymorph, a pharmaceutically acceptable salt, a prodrug, a hydrate, or a solvate thereof;

wherein

Z₁ and Z₂ are each selected from N and C whereby if Z₁ is N, Z₂ is C, and if Z₁ is C, Z₂ is N;

V, X and E are each independently selected from N and CRₐ;

wherein at least one of V, X and E is CRₐ;

Rₐ is independently selected from the group consisting of —H, —CF₃ and halo;

R₀ is selected from the group consisting of —H, C₁-C₃alkyl and halo;

R₁ is selected from the group consisting of —CF₃, —OCF₃, —OCHF₂ and halo;

R₂ is selected from the group consisting of —OH, —H and —CF₃;

Y is selected from NH and O; and

R₃ is selected from the group consisting of

4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄alkyl, haloC₁-C₄alkyl, hydroxyC₁-C₄alkyl, —OH and halo;

5- or 6-membered aryl or heteroaryl, wherein heteroaryl contains one or two heteroatoms, wherein said heteroatom(s) is/are N, optionally substituted with one or two substituents independently selected from the group consisting of C₁-C₄alkyl, hydroxyC₁-C₄alkyl, haloC₁-C₄alkyl, —CN and halo;

C₃-C₆cycloalkyl optionally substituted with one or two substituents independently selected from the group consisting of C₁-C₄ alkyl, haloC₁-C₄ alkyl, hydroxyC₁-C₄ alkyl, —OH and halo; and

C₁-C₆alkyl substituted with one or two substituents independently selected from the group consisting of —OH, halo, haloC₁-C₄alkyl and C₁-C₄alkoxy.

Within a preferred aspect, provided herein is a compound of formula (I′)

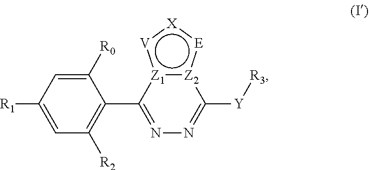

or a stereoisomer, a racemic mixture, a tautomer, a polymorph, a pharmaceutically acceptable salt, a prodrug, a hydrate, or a solvate thereof;

wherein

Z₁ is N;

Z₂ is C;

V, X and E are each independently selected from N and CRₐ;

wherein at least one of V, X and E is CRₐ;

Rₐ is independently selected from the group consisting of —H, —CF₃ and halo;

R₀ is selected from the group consisting of —H, C₁-C₃alkyl and halo;

R₁ is selected from the group consisting of —CF₃, —OCF₃, —OCHF₂ and halo;

R₂ is selected from the group consisting of —OH, —H and —CF₃;

Y is selected from NH and O;

R₃ is selected from the group consisting of

4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄alkyl, haloC₁-C₄alkyl, hydroxyC₁-C₄alkyl, —OH and halo;

5- or 6-membered aryl or heteroaryl, wherein heteroaryl contains one or two heteroatoms, wherein said heteroatom(s) is/are N, optionally substituted with one or two substituents independently selected from the group consisting of Cratalkyl, hydroxyC₁-C₄alkyl, haloC₁-C₄alkyl, —CN and halo;

C₃-C₆cycloalkyl substituted with one or two substituents independently selected from the group consisting of C₁-C₄alkyl, haloC₁-C₄alkyl, hydroxyC₁-C₄ alkyl, —OH and halo; and

C₁-C₆alkyl substituted with one or two substituents independently selected from the group consisting of —OH, halo, haloC₁-C₄alkyl and C₁-C₄alkoxy.

In one aspect there is provided a compound of formula (Ib)

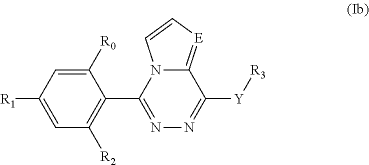

or a stereoisomer, a racemic mixture, a tautomer, a polymorph, a pharmaceutically acceptable salt, a prodrug, a hydrate, or a solvate thereof;

wherein

E is selected from N and CRₐ;

Rₐ is selected from the group consisting of —H, -C₁-C₃alkyl, —CF₃ and halo;

R₀ is selected from the group consisting of —H, C₁-C₃alkyl and halo;

R₁ is selected from the group consisting of —CF₃, —OCF₃, —OCHF₂ and halo;

R₂ is selected from the group consisting of —OH, —H and —CF₃;

Y is selected from NH and O; and

R₃ is selected from the group consisting of

4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄alkyl, haloC₁-C₄alkyl, hydroxyC₁-C₄alkyl, —OH and halo;

5- or 6-membered aryl or heteroaryl, wherein heteroaryl contains one or two heteroatoms, wherein said heteroatom(s) is/are N, optionally substituted with one or two substituents independently selected from the group consisting of C₁-C₄alkyl, hydroxyC₁-C₄alkyl, haloC₁-C₄alkyl, —CN and halo; C₃-C₆cycloalkyl optionally substituted with one or two substituents independently selected from the group consisting of C₁-C₄alkyl, haloC₁-C₄alkyl, hydroxyC₁-C₄alkyl, —OH and halo; and

C₁-C₆ alkyl substituted with one or two substituents independently selected from the group consisting of —OH, halo, haloC₁-C₄ alkyl and C₁-C₄alkoxy.

In another aspect there is provided a compound of formula (Ic)

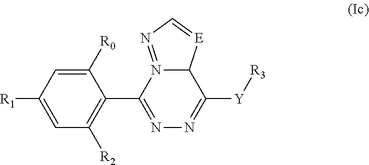

or a stereoisomer, a racemic mixture, a tautomer, a polymorph, a pharmaceutically acceptable salt, a prodrug, a hydrate, or a solvate thereof;

wherein

E is selected from N and CRₐ;

Rₐ is selected from the group consisting of —H, -C₁-C₃alkyl, —CF₃ and halo;

R₀ is selected from the group consisting of —H, C₁-C₃alkyl and halo;

R₁ is selected from the group consisting of —CF₃, —OCF₃, —OCHF₂ and halo;

R₂ is selected from the group consisting of —OH, —H and —CF₃;

Y is selected from NH and O; and

R₃ is selected from the group consisting of

4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄alkyl, haloC₁-C₄ alkyl, hydroxyC₁-C₄alkyl, —OH and halo;

5- or 6-membered aryl or heteroaryl, wherein heteroaryl contains one or two heteroatoms, wherein said heteroatom(s) is/are N, optionally substituted with one or two substituents independently selected from the group consisting of C₁-C₄alkyl, hydroxyC₁-C₄alkyl, haloC₁-C₄alkyl, —CN and halo;

C₃-C₆ cycloalkyl optionally substituted with one or two substituents independently selected from the group consisting of C₁-C₄alkyl, haloC₁-C₄ alkyl, hydroxyC₁-C₄alkyl, —OH and halo; and

C₁-C₆ alkyl substituted with one or two substituents independently selected from the group consisting of —OH, halo, haloC₁-C₄alkyl and C₁-C₄alkoxy.

In another aspect there is provided a compound of formula (Ie)

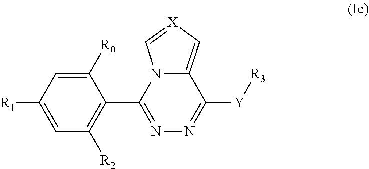

or a stereoisomer, a racemic mixture, a tautomer, a polymorph, a pharmaceutically acceptable salt, a prodrug, a hydrate, or a solvate thereof;

wherein

X is selected from N and CRₐ;

Rₐ is selected from the group consisting of -C₁-C₃alkyl, —CF₃ and halo;

R₀ is selected from the group consisting of —H, C₁-C₃alkyl and halo;

R₁ is selected from the group consisting of —CF₃, —OCF₃, —OCHF₂ and halo;

R₂ is selected from the group consisting of —OH, —H and —CF₃;

Y is selected from NH and O; and

R₃ is selected from the group consisting of

4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄alkyl, haloC₁-C₄ alkyl, hydroxyC₁-C₄ alkyl, —OH and halo;

4-, 5- or 6-membered aryl or heteroaryl, wherein heteroaryl contains one or two heteroatoms, wherein said heteroatom(s) is/are N, optionally substituted with one or two substituents independently selected from the group consisting of C₁-C₄alkyl, hydroxyC₁-C₄alkyl, haloC₁-C₄alkyl, —CN and halo;

C₃-C₆ cycloalkyl optionally substituted with one or two substituents independently selected from the group consisting of C₁-C₄alkyl, haloC₁-C₄alkyl, hydroxyC₁-C₄alkyl, —OH and halo; and

C₁-C₆alkyl substituted with one or two substituents independently selected from the group consisting of —OH, halo, haloC₁-C₄alkyl and C₁-C₄alkoxy.

Within one aspect, there is provided a compound of formula (Ia)

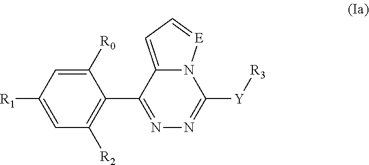

or a stereoisomer, a racemic mixture, a tautomer, a polymorph, a pharmaceutically acceptable salt, a prodrug, a hydrate, or a solvate thereof;

wherein

E is selected from N and CRₐ;

Rₐ is selected from the group consisting of —H, -C₁-C₃alkyl, and —CF₃;

R₀ is selected from the group consisting of —H, C₁-C₃alkyl and halo;

R₁ is selected from the group consisting of —CF₃, —OCF₃, —OCHF₂ and halo;

R₂ is selected from the group consisting of —OH, —H and —CF₃;

Y is selected from NH and O; and

R₃ is selected from the group consisting of

4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄ alkyl, haloC₁-C₄alkyl, hydroxyC₁-C₄alkyl, —OH and halo;

4-, 5- or 6-membered aryl or heteroaryl, wherein heteroaryl contains one or two heteroatoms, wherein said heteroatom(s) is/are N, optionally substituted with one or two substituents independently selected from the group consisting of C₁-C₄alkyl, hydroxyC₁-C₄ alkyl, haloC₁-C₄alkyl, —CN and halo;

C₃-C₆cycloalkyl optionally substituted with one or two substituents independently selected from the group consisting of C₁-C₄alkyl, haloC₁-C₄ alkyl, hydroxyC₁-C₄ alkyl, —OH and halo; and

C₁-C₆ alkyl substituted with one or two substituents independently selected from the group consisting of —OH, halo, haloC₁-C₄ alkyl and C₁-C₄alkoxy.

In another aspect there is provided a compound of formula (Id)

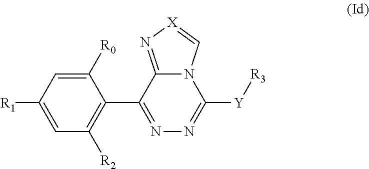

or a stereoisomer, a racemic mixture, a tautomer, a polymorph, a pharmaceutically acceptable salt, a prodrug, a hydrate, or a solvate thereof;

wherein

X is selected from N and CRₐ;

Rₐ is selected from the group consisting of —H, -C₁-C₃alkyl, —CF₃ and halo;

R₀ is selected from the group consisting of —H, C₁-C₃alkyl and halo;

R₁ is selected from the group consisting of —CF₃, —OCF₃, —OCHF₂ and halo;

R₂ is selected from the group consisting of —OH, —H and —CF₃;

Y is selected from NH and O; and

R₃ is selected from the group consisting of

4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄alkyl, haloC₁-C₄alkyl, hydroxyC₁-C₄ alkyl, —OH and halo;

4-, 5- or 6-membered aryl or heteroaryl, wherein heteroaryl contains one or two heteroatoms, wherein said heteroatom(s) is/are N, optionally substituted with one or two substituents independently selected from the group consisting of C₁-C₄alkyl, hydroxyC₁-C₄alkyl, haloC₁-C₄alkyl, —CN and halo;

C₃-C₆ cycloalkyl optionally substituted with one or two substituents independently selected from the group consisting of C₁-C₄ alkyl, haloC₁-C₄ alkyl, hydroxyC₁-C₄ alkyl, —OH and halo; and C₁-C₆alkyl substituted with one or two substituents independently selected from the group consisting of —OH, halo, haloC₁-C₄alkyl and C₁-C₄alkoxy.

Within the present invention any reference to the compounds of formula (I′), (I), (Ia), (Ib), (Ic), (Id) or (Ie), or the preferred embodiments thereof is intended to also refer to the stereoisomers, or racemic mixtures, or tautomers, or polymorphs, or pharmaceutically acceptable salts, or prodrugs, or hydrates, or solvates thereof.

Compounds of formula (I′), (I), (Ia), (Ib), (Ic), (Id) or (Ie), or a stereoisomer, a racemic mixture, a tautomer, a polymorph, a pharmaceutically acceptable salt, a prodrug, a hydrate, or a solvate thereof, are suitable for the treatment, alleviation or prevention of a disease, disorder or an abnormality which is responsive to the modulation, in particular inhibition, of a component of the NLRP3 inflammasome pathway, or which is responsive to the modulation, in particular decrease, of IL-1 beta and/or IL-18 levels. In particular, the component of the inflammasome pathway is the NLRP3 inflammasome. Activation of the NLRP3 inflammasome pathway can trigger the formation of ASC specks, cleavage and activation of Caspase-1 and Caspase-8 and subsequent activation and release IL-1 beta, IL-18, gasdermin D cleavage and pore formation, pyroptosis, and release of IL-1alpha, IL-33, IL-17 and High-Mobility Group Box (HMGB) protein. The compounds of formula (I′), (I), (Ia), (Ib), (Ic), (Id) or (Ie), or a stereoisomer, a racemic mixture, a tautomer, a polymorph, a pharmaceutically acceptable salt, a prodrug, a hydrate, or a solvate thereof, have the capability to modulate, in particular decrease, IL-1 beta and/or IL-18 levels.

The compounds of formula (I′), (I), (Ia), (Ib), (Ic), (Id) or (Ie), or a stereoisomer, a racemic mixture, a tautomer, a polymorph, a pharmaceutically acceptable salt, a prodrug, a hydrate, or a solvate thereof, display high capability in modulating and, in particular inhibiting the activation of, a component of the NLRP3 inflammasome pathway, in particular wherein the component of the inflammasome pathway is the NLRP3 inflammasome. Due to their unique design features, these compounds display properties such as modulating or inhibiting the activation of the NLRP3 inflammasome pathway allowing them to be a successful medicament for the treatment, alleviation or prevention of diseases, disorders and abnormalities responsive to the modulation or inhibition of a component of the NLRP3 inflammasome pathway such as, for example, Alzheimer's disease, Parkinson's disease, CAPS, non-alcoholic fatty liver disease (NAFLD), non-alcoholic steatohepatitis (NASH) and gout.

In a further embodiment, the invention relates to a pharmaceutical composition comprising a compound of formula (I′), (I), (Ia), (Ib), (Ic), (Id) or (Ie), or a stereoisomer, a racemic mixture, a tautomer, a polymorph, a pharmaceutically acceptable salt, a prodrug, a hydrate, or a solvate thereof, and optionally comprising at least one pharmaceutically acceptable carrier, diluent, adjuvant or excipient.

In another embodiment, the present invention refers to a compound of formula (I′), (I), (Ia), (Pb), (Ic), (Id) or (Ie), or a stereoisomer, a racemic mixture, a tautomer, a polymorph, a pharmaceutically acceptable salt, a prodrug, a hydrate, or a solvate thereof, for use as a medicament.

Yet another embodiment, the present invention refers to a compound of formula (I′), (I), (Ia), (Ib), (Ic), (Id) or (Ie), or a stereoisomer, a racemic mixture, a tautomer, a polymorph, a pharmaceutically acceptable salt, a prodrug, a hydrate, or a solvate thereof, for use in the treatment, alleviation or prevention of a disease, disorder, or abnormality which is responsive to the modulation, in particular inhibition of activation, of a component of the NLRP3 inflammasome pathway and/or which is responsive to the modulation, in particular decrease, of IL-1 beta and/or IL-18 levels.

A further embodiment is concerned with the use of the compound of formula (I′), (I), (Ia), (Ib), (Ic), (Id) or (Ie), or a stereoisomer, a racemic mixture, a tautomer, a polymorph, a pharmaceutically acceptable salt, a prodrug, a hydrate, or a solvate thereof, for the manufacture of a medicament for treating, alleviating or preventing a disease, disorder or abnormality which is responsive to the modulation, in particular inhibition of activation, of a component of the NLRP3 inflammasome pathway and/or which is responsive to the modulation, in particular decrease, of IL-1 beta and/or IL-18 levels.

In yet another embodiment, the present invention is directed to a method of treating, alleviating or preventing a disease, disorder or abnormality which is responsive to the modulation, in particular inhibition of activation, of a component of the NLRP3 inflammasome pathway or which is responsive to the modulation, in particular decrease, of IL-1 beta and/or IL-18 levels, the method comprising administering a therapeutically effective amount of a compound of formula (I′), (I), (Ia), (Ib), (Ic), (Id) or (Ie), or a stereoisomer, a racemic mixture, a tautomer, a polymorph, a pharmaceutically acceptable salt, a prodrug, a hydrate, or a solvate thereof, to a subject in need thereof (e.g. a patient).

A pharmaceutical composition comprising a combination of a compound of formula (I′), (I), (Ia), (Ib), (Ic), (Id) or (Ie), or a stereoisomer, a racemic mixture, a tautomer, a polymorph, a pharmaceutically acceptable salt, a prodrug, a hydrate, or a solvate thereof, and at least one further biologically active compound differing from the compound of formula (I′), (I), (Ia), (Ib), (Ic), (Id) or (Ie), or a stereoisomer, a racemic mixture, a tautomer, a polymorph, a pharmaceutically acceptable salt, a prodrug, a hydrate, or a solvate thereof, and optionally comprising at least one pharmaceutically acceptable carrier, diluent, adjuvant or excipient, is also the subject-matter of the present invention.

In particular, the further biologically active compound can be one which is used for the treatment of a disease, disorder, or abnormality associated with a disease targeting different pathomechanism, e.g. an anti-amyloid beta antibody, anti-Tau antibody, amyloid beta small molecule inhibitor, Tau aggregation small molecule inhibitor, anti-alpha synuclein antibody or alpha-synuclein aggregation small molecule inhibitor, anti-TDP-43 antibody or anti-TDP-43 aggregation small molecule inhibitor, among others. When a compound of the invention is used in combination with a further biologically active compound, the dose of each compound may differ from the dose if the compound is used as monotherapy.

An additional embodiment relates to the use of the compound of formula (I′), (I), (Ia), (Pb), (Ic), (Id) or (Ie), or a stereoisomer, a racemic mixture, a tautomer, a polymorph, a pharmaceutically acceptable salt, a prodrug, a hydrate, or a solvate thereof, as an analytical reference or an in vitro screening tool.

The present invention is described hereinafter.

### BRIEF DESCRIPTION OF THE FIGURE

FIG. 1 Inhibition of IL-1beta release by a compound of the invention in LPS/ATP induced acute peritonitis mouse model

### DETAILED DESCRIPTION OF THE INVENTION

The present invention relates to compounds of formula (I′), (I), (Ia), (Ib), (Ic), (Id) or (Ie) including stereoisomers, racemic mixtures, tautomers, polymorphs, pharmaceutically acceptable salts, prodrugs, hydrates, or solvates thereof.

Any of the definitions of R₀, R₁, R₂, R₃, Rₐ, E, V, X and Y which are given with respect to the compounds of formula (I) apply analogously to compounds of formula (I′), (Ia), (Pb), (Ic), (Id), and (Ie) wherever R₀, R₁, R₂, R₃, Rₐ, E, V, X and/or Y are used.

The present invention relates to compounds of formula (I′) as defined below

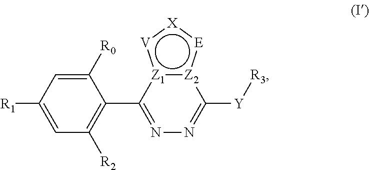

or a stereoisomer, a racemic mixture, a tautomer, a polymorph, a pharmaceutically acceptable salt, a prodrug, a hydrate, or a solvate thereof;

wherein

Z₁ is N;

Z₂ is C;

V, X and E are each independently selected from N and CRₐ;

wherein at least one of V, X and E is CRₐ;

Rₐ is independently selected from the group consisting of —H, -C₁-C₃alkyl, —CF₃ and halo;

R₀ is selected from the group consisting of —H, C₁-C₃alkyl and halo;

R₁ is selected from the group consisting of —CF₃, —OCF₃, —OCHF₂ and halo;

R₂ is selected from the group consisting of —OH, —H and —CF₃;

Y is selected from NH and O;

R₃ is selected from the group consisting of

4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄ alkyl, haloC₁-C₄ alkyl, hydroxyC₁-C₄alkyl, —OH and halo;

5- or 6-membered aryl or heteroaryl, wherein heteroaryl contains one or two heteroatoms, wherein said heteroatom(s) is/are N, optionally substituted with one or two substituents independently selected from the group consisting of C₁-C₄ alkyl, hydroxyC₁-C₄alkyl, haloC₁-C₄alkyl, —CN and halo;

C₃-C₆cycloalkyl optionally substituted with one or two substituents independently selected from the group consisting of C₁-C₄ alkyl, haloC₁-C₄alkyl, hydroxyC₁-C₄ alkyl, —OH and halo; and

C₁-C₆alkyl substituted with one or two substituents independently selected from the group consisting of —OH, halo, haloC₁-C₄alkyl and C₁-C₄alkoxy.

The present invention relates to compounds of formula (I) as defined below

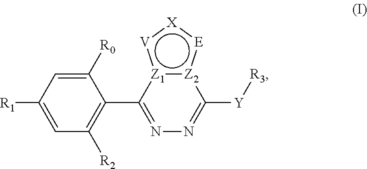

or a stereoisomer, a racemic mixture, a tautomer, a polymorph, a pharmaceutically acceptable salt, a prodrug, a hydrate, or a solvate thereof;

wherein

Z₁ and Z₂ are each selected from N and C whereby if Z₁ is N, Z₂ is C, and if Z₁ is C, Z₂ is N;

V, X and E are each independently selected from N and CRₐ;

wherein at least one of V, X and E is CRₐ;

Rₐ is selected from the group consisting of —H, -C₁-C₃alkyl, —CF₃ and halo;

R₀ is selected from the group consisting of —H, C₁-C₃alkyl and halo;

R₁ is selected from the group consisting of —CF₃, —OCF₃, —OCHF₂ and halo;

R₂ is selected from the group consisting of —OH, —H and —CF₃;

Y is selected from NH and O; and

R₃ is selected from the group consisting of

4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄ alkyl, haloC₁-C₄alkyl, hydroxyC₁-C₄ alkyl, —OH and halo;

5- or 6-membered aryl or heteroaryl, wherein heteroaryl contains one or two heteroatoms, wherein said heteroatom(s) is/are N, optionally substituted with one or two substituents independently selected from the group consisting of C₁-C₄alkyl, hydroxyC₁-C₄alkyl, haloC₁-C₄alkyl, —CN and halo;

C₃-C₆cycloalkyl optionally substituted with one or two substituents independently selected from the group consisting of C₁-C₄alkyl, haloC₁-C₄alkyl, hydroxyC₁-C₄ alkyl, —OH and halo; and C₁-C₆ alkyl substituted with one or two substituents independently selected from the group consisting of —OH, halo, haloC₁-C₄alkyl and C₁-C₄alkoxy.

In a preferred embodiment, R₀ is selected from —H and C₁-C₃alkyl. In one preferred embodiment R₀ is —H. In another preferred embodiment R₀ is C₁-C₃alkyl, preferably methyl or ethyl, more preferably methyl.

In a preferred embodiment, R₁ is selected from —CF₃, —OCF₃ and —OCHF₂. In one preferred embodiment R₁ is —CF₃. In another preferred embodiment R₁ is —OCF₃. In another preferred embodiment R₁ is —OCHF₂.

In another embodiment, R₁ is halo. Where R₁ is halo, the halogen (halo) is preferably chloro.

In one preferred embodiment, R₂ is —OH or H, more preferably —OH. In another embodiment R₂ is —H.

In another embodiment R₂ is —CF₃.

In one preferred embodiment, R₀ is —H, R₁ is selected from —CF₃, —OCF₃ and —OCHF₂, and R₂ is —OH or —H. In one preferred embodiment R₀ is —H, R₁ is —CF₃, and R₂ is —OH.

In preferred embodiments, R₃ is selected from the group consisting of

4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄alkyl, haloC₁-C₄alkyl, hydroxyC₁-C₄alkyl, —OH and halo;

6-membered aryl or heteroaryl, wherein heteroaryl contains one or two heteroatoms, wherein said heteroatom(s) is/are N, optionally substituted with one or two substituents independently selected from the group consisting of C₁-C₄alkyl, hydroxyC₁-C₄alkyl, haloC₁-C₄alkyl, —CN and halo;

C₃-C₆ cycloalkyl optionally substituted with one or two substituents independently selected from the group consisting of C₁-C₄ alkyl, haloC₁-C₄ alkyl, hydroxyC₁-C₄ alkyl, —OH and halo; and

C₁-C₆ alkyl substituted with one or two substituents independently selected from the group consisting of —OH, halo, haloC₁-C₄alkyl and C₁-C₄alkoxy.

R₃ is selected from the group consisting of

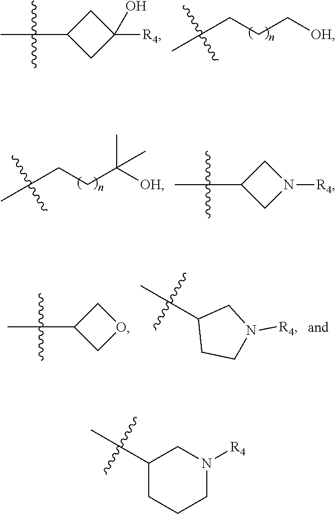

wherein R₄ is independently selected from —H or -C₁-C₃alkyl; and

n is selected from 0, 1 or 2.

Preferably, R₃ is selected from the group consisting of

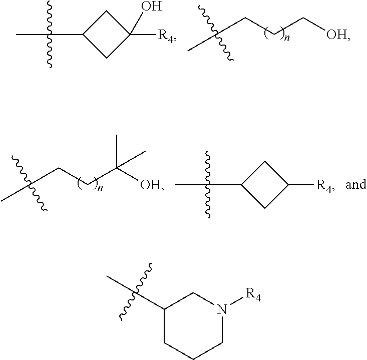

wherein R₄ is independently selected from —H or -C₁-C₃alkyl; and

n is selected from 0, 1 or 2.

In one preferred embodiment, R₃ is selected from a 5- or 6-membered heterocycloalkyl containing one or two heteroatoms, preferably one heteroatom, wherein said heteroatom(s) is/are N. In one preferred embodiment R₃ is a 6-membered heterocycloalkyl containing one heteroatom, wherein said heteroatom is N. The 5- or 6-membered heterocycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the heterocyclic group.

In one preferred embodiment, R₃ is

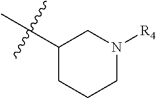

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In one preferred embodiment, R₃ is

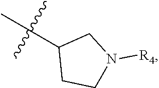

wherein R₄ is independently selected from —H or C₁-C₃alkyl. In a preferred embodiment R₄ is H. In another embodiment, R₄ is C₁-C₃alkyl, for instance methyl, ethyl or propyl. In a preferred embodiment, R₄ is methyl.

In another preferred embodiment, R₃ is

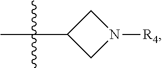

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl, whereby R₃ is

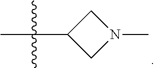 [SMILES: *C1CN(C)C1]

In a further preferred embodiment, R₃ is

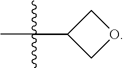 [SMILES: *C1COC1]

In another embodiment R₃ is selected from a 6-membered aryl or heteroaryl, wherein heteroaryl contains one or two heteroatoms, wherein said heteroatom(s) is/are N. The 6-membered aryl or heteroaryl can be substituted with one or two substituents independently selected from the group consisting of C₁-C₄alkyl, hydroxyC₁-C₄alkyl, haloC₁-C₄ alkyl, —CN and halo. The substituents can be at any available position on the aryl or heteroaryl group.

In one embodiment R₃ is a 6-membered heteroaryl with two N heteroatoms. The 6-membered heteroaryl can be substituted with one or two substituents independently selected from the group consisting of C₁-C₄alkyl, hydroxyC₁-C₄ alkyl, haloC₁-C₄alkyl, —CN and halo. In one embodiment R₃ is selected from the group consisting of

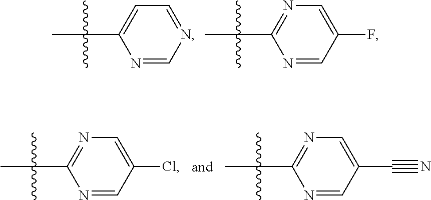 [SMILES: *c1ccncn1 ; *c1ncc(F)cn1 ; *c1ncc(Cl)cn1 ; *c1ncc(CN)cn1]

In another embodiment R₃ is a C₃-C₆ cycloalkyl, preferably cyclopropyl or cyclobutyl. The C₃-C₆ cycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the cycloalkyl group.

In one preferred embodiment R₃ is

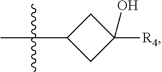

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl. In one preferred embodiment R₃ is

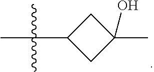 [SMILES: *C1CC(C)(O)C1]

In another preferred embodiment R₃ is

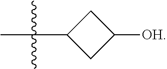 [SMILES: *C1CC(O)C1]

In another preferred embodiment R₃ is from C₁-C₆ alkyl, preferably C₁-C₃alkyl, for example methyl, ethyl or propyl. The alkyl can be substituted with one or two substituents independently selected from the group consisting of —OH, halo, haloC₁-C₄alkyl and C₁-C₄alkoxy. In one preferred embodiment R₃ is hydroxyC₁-C₆alkyl (i.e. C₁-C₆alkyl substituted with —OH), more preferably hydroxyC₁-C₃alkyl, preferable hydroxyethyl.

In one preferred embodiment R₃ is

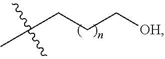

wherein n is 0, 1 or 2. In another preferred embodiment R₃ is

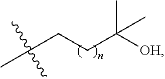

wherein n is 0, 1 or 2.

In some embodiments one of V, X and E is CRₐ, wherein Rₐ is as defined above. In a preferred embodiment two out of V, X and E are CRₐ, wherein Rₐ is as defined above. In another preferred embodiment V, X and E are each CRₐ, wherein Rₐ is as defined above.

In one preferred embodiment X is CRₐ, wherein Rₐ is selected from H and -C₁-C₃alkyl. In a preferred embodiment X is CH.

In a preferred embodiment Z₁ is N. In one preferred embodiment Z₁ is N and V is CH and X and E are independently selected from N and CRₐ, wherein Rₐ is as defined above.

In another preferred embodiment Z₁ is N and V, X and E are all CRₐ, wherein Rₐ is as defined above.

In some preferred embodiments Z₁ is N and at least two of V, X, and E are CRₐ, wherein Rₐ is as defined above. In one preferred embodiment Z₁ is N and V, X and E are all CH.

In another preferred embodiment Z₁ is N, E is N and V and X are each CRₐ, wherein Rₐ is as defined above. In one embodiment Z₁ is N, E is N and V and X are both CH. In another preferred embodiment

Z₁ is N, V is N and E and X are each CRₐ, wherein Rₐ is as defined above. In one embodiment Z₁ is N, V is N and E and X are both CH. In another embodiment Z₁ is N, X is N and V and E are each CRₐ, wherein Rₐ is as defined above. In another embodiment Z₁ is N, X is N and V and E are both CH.

In a preferred embodiment, Z₁ is N and Z₂ is C.

In more preferred embodiments:

Z₁ is N, V is N, X and E are CRₐ and Z₂ is C; or

Z₁ is N and V, X, E are CRₐ and Z₂ is C; or

Z₁ is N and E is N, X and V are CRₐ and Z₂ is C.

In other aspects Z₂ is N. In one embodiment Z₂ is N and E is CH and V and X are independently selected from N and CRₐ, wherein Rₐ. In one embodiment Z₂ is N and V, X and E are all CRₐ, wherein Rₐ is as defined above. In some embodiments Z₂ is N and at least two of V, X, and E are CRₐ, wherein

Rₐ is as defined above. In one embodiment Z₂ is N and V, X and E are all CH. In another embodiment Z₂ is N, E is N and V and X are each CRₐ, wherein Rₐ is as defined above. In one embodiment Z₂ is N, E is N and V and X are both CH. In another embodiment Z₂ is N, V is N and E and X are each CRₐ, wherein Rₐ is as defined above. In one embodiment Z₂ is N, V is N and E and X are both CH.

In another embodiment Z₂ is N, X is N and V and E are each CRₐ, wherein Rₐ is as defined above.

In another embodiment Z₂ is N, X is N and V and E are both CH.

In an embodiment there is provided a compound of formula (I) having the formula (Ia):

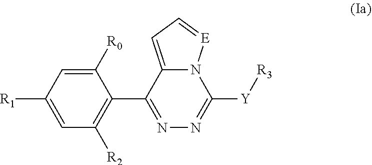

or a stereoisomer, a racemic mixture, a tautomer, a polymorph, a pharmaceutically acceptable salt, a prodrug, a hydrate, or a solvate thereof;

wherein

E is selected from N and CRₐ; Rₐ is selected from the group consisting of —H, -C₁-C₃alkyl, and —CF₃, and R₀, R₁, R₂, R₃, and Y are as defined in any of the above embodiments.

In one preferred embodiment of the compound of formula (Ia) E is N. In another preferred embodiment of the compound of formula (Ia) E is CRₐ, wherein Rₐ is as defined above. In one preferred embodiment of the compound of formula (Ia) E is CH.

In a preferred embodiment R₀ is selected from —H and C₁-C₃alkyl. In one preferred embodiment R₀ is —H. In another preferred embodiment R₀ is C₁-C₃alkyl, preferably methyl or ethyl, more preferably methyl.

In a preferred embodiment, R₁ is selected from —CF₃, —OCF₃ and —OCHF₂. In one preferred embodiment R₁ is —CF₃. In another preferred embodiment R₁ is —OCF₃. In another preferred embodiment R₁ is —OCHF₂.

In another embodiment R₁ is halo. Where R₁ is halo, the halogen (halo) is preferably chloro.

In one preferred embodiment R₂ is —OH. In another embodiment R₂ is —H. In another embodiment R₂ is —CF₃.

In one preferred embodiment R₀ is —H, R₁ is selected from —CF₃, —OCF₃ and —OCHF₂, and R₂ is —OH or —H. In one preferred embodiment R₀ is —H, R₁ is —CF₃, and R₂ is —OH.

In preferred embodiments R₃ is independently selected from the group consisting of 4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄alkyl, haloC₁-C₄alkyl, hydroxyC₁-C₄alkyl, —OH and halo;

6-membered aryl or heteroaryl, wherein heteroaryl contains one or two heteroatoms, wherein said heteroatom(s) is/are N, optionally substituted with one or two substituents independently selected from the group consisting of C₁-C₄alkyl, hydroxyC₁-C₄alkyl, haloC₁-C₄alkyl, —CN and halo; C₃-C₆ cycloalkyl optionally substituted with one or two substituents independently selected from the group consisting of C₁-C₄ alkyl, haloC₁-C₄alkyl, hydroxyC₁-C₄ alkyl, —OH and halo; and

C₁-C₆alkyl substituted with one or two substituents independently selected from the group consisting of —OH, halo, haloC₁-C₄ alkyl and C₁-C₄alkoxy.

In one preferred embodiment R₃ is selected from a 5- or 6-membered heterocycloalkyl containing one or two heteroatoms, preferably one heteroatom, wherein said heteroatom(s) is/are N. In one preferred embodiment R₃ is a 6-membered heterocycloalkyl containing one heteroatom, wherein said heteroatom is N. The 5- or 6-membered heterocycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the heterocyclic group.

In one preferred embodiment R₃ is

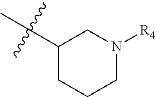

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In one preferred embodiment R₃ is

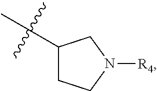

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In a further preferred embodiment, R₃ is

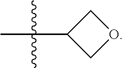 [SMILES: *C1COC1]

In another preferred embodiment R₃ is

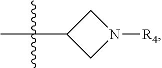

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl, whereby R₃ is

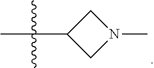 [SMILES: *C1CN(C)C1]

In another embodiment R₃ is selected from a 6-membered aryl or heteroaryl, wherein heteroaryl contains one or two heteroatoms, wherein said heteroatom(s) is/are N. The 6-membered aryl or heteroaryl can be substituted with one or two substituents independently selected from the group consisting of C₁-C₄alkyl, hydroxyC₁-C₄alkyl, haloC₁-C₄alkyl, —CN and halo. The substituents can be at any available position on the aryl or heteroaryl group.

In one embodiment R₃ is a 6-membered heteroaryl with two N heteroatoms. The 6-membered heteroaryl can be substituted with one or two substituents independently selected from the group consisting of C₁-C₄alkyl, hydroxyC₁-C₄alkyl, haloC₁-C₄alkyl, —CN and halo. In one embodiment R₃ is selected from the group consisting of

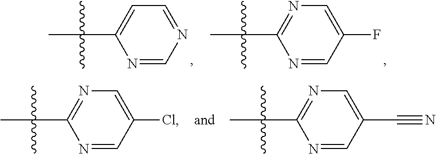

In another embodiment R₃ is a C₃-C₆cycloalkyl, preferably cyclopropyl or cyclobutyl. The C₃-C₆ cycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the cycloalkyl group.

In one preferred embodiment R₃ is

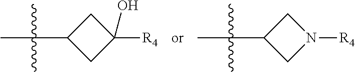

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl. In one preferred embodiment R₃ is

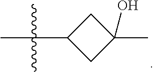 [SMILES: *C1CC(C)(O)C1]

In another preferred embodiment R₃ is

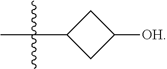 [SMILES: *C1CC(O)C1]

In another preferred embodiment R₃ is from C₁-C₆alkyl, preferably C₁-C₃alkyl, for example methyl, ethyl or propyl. The alkyl can be substituted with one or two substituents independently selected from the group consisting of —OH, halo, haloC₁-C₄alkyl and C₁-C₄alkoxy. In one preferred embodiment R₃ is hydroxyC₁-C₆alkyl (i.e. C₁-C₆ alkyl substituted with —OH), more preferably hydroxyC₁-C₃alkyl, preferable hydroxyethyl.

In one preferred embodiment R₃ is

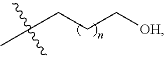

wherein n is 0, 1 or 2. In another preferred embodiment R₃ is

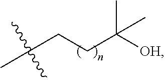

wherein n is 0, 1 or 2.

In a preferred embodiment there is provided a compound of formula (I) or (I′), having the formula (Ib)

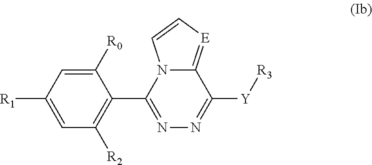

or a stereoisomer, a racemic mixture, a tautomer, a polymorph, a pharmaceutically acceptable salt, a prodrug, a hydrate, or a solvate thereof;

wherein Rₐ, R₀, R₁, R₂, R₃, E and Y are as defined in any of the above embodiments.

In one preferred embodiment of the compound of formula (Ib) E is N. In another preferred embodiment of the compound of formula (Ib) E is CRₐ, wherein Rₐ is as defined above. In one preferred embodiment of the compound of formula (Ib) E is CH.

In a preferred embodiment R₀ is selected from —H and C₁-C₃alkyl. In one preferred embodiment R₀ is —H. In another preferred embodiment R₀ is C₁-C₃alkyl, preferably methyl or ethyl, more preferably methyl.

In a preferred embodiment, R₁ is selected from —CF₃, —OCF₃ and —OCHF₂. In one preferred embodiment R₁ is —CF₃. In another preferred embodiment R₁ is —OCF₃. In another preferred embodiment R₁ is —OCHF₂.

In another embodiment R₁ is halo. Where R₁ is halo, the halogen (halo) is preferably chloro.

In one preferred embodiment R₂ is —OH. In another embodiment R₂ is —H. In another embodiment R₂ is —CF₃.

In one preferred embodiment R₀ is —H, R₁ is selected from —CF₃, —OCF₃ and —OCHF₂, and R₂ is —OH or —H. In one preferred embodiment R₀ is —H, R₁ is —CF₃, and R₂ is —OH.

In preferred embodiments R₃ is independently selected from the group consisting of 4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄alkyl, haloC₁-C₄alkyl, hydroxyC₁-C₄ alkyl, —OH and halo;

6-membered aryl or heteroaryl, wherein heteroaryl contains one or two heteroatoms, wherein said heteroatom(s) is/are N, optionally substituted with one or two substituents independently selected from the group consisting of C₁-C₄alkyl, hydroxyC₁-C₄alkyl, haloC₁-C₄alkyl, —CN and halo; C₃-C₆ cycloalkyl optionally substituted with one or two substituents independently selected from the group consisting of C₁-C₄ alkyl, haloC₁-C₄ alkyl, hydroxyC₁-C₄ alkyl, —OH and halo; and C₁-C₆ alkyl substituted with one or two substituents independently selected from the group consisting of —OH, halo, haloC₁-C₄alkyl and C₁-C₄alkoxy.

In one preferred embodiment R₃ is selected from a 5- or 6-membered heterocycloalkyl containing one or two heteroatoms, preferably one heteroatom, wherein said heteroatom(s) is/are N. In one preferred embodiment R₃ is a 6-membered heterocycloalkyl containing one heteroatom, wherein said heteroatom is N. The 5- or 6-membered heterocycloalkyl can be substituted with one or two substituents independently selected from the group consisting of C₁-C₄alkyl, haloC₁-C₄ alkyl, hydroxyC₁-C₄alkyl, —OH and halo. The substituents can be at any available position on the heterocyclic group.

In one preferred embodiment R₃ is

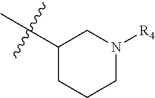

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In one preferred embodiment R₃ is

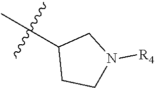

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In a further preferred embodiment, R₃ is

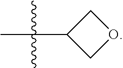 [SMILES: *C1COC1]

In another preferred embodiment R₃ is

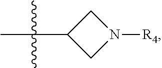

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl, whereby R₃ is

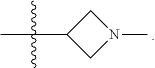 [SMILES: *C1CN(C)C1]

In another embodiment R₃ is selected from a 6-membered aryl or heteroaryl, wherein heteroaryl contains one or two heteroatoms, wherein said heteroatom(s) is/are N. The 6-membered aryl or heteroaryl can be substituted with one or two substituents independently selected from the group consisting of C₁-C₄ alkyl, hydroxyC₁-C₄alkyl, haloC₁-C₄ alkyl, —CN and halo. The substituents can be at any available position on the aryl or heteroaryl group.

In one embodiment R₃ is a 6-membered heteroaryl with two N heteroatoms. The 6-membered heteroaryl can be substituted with one or two substituents independently selected from the group consisting of C₁-C₄alkyl, hydroxyC₁-C₄alkyl, haloC₁-C₄alkyl, —CN and halo. In one embodiment R₃ is selected from the group consisting of

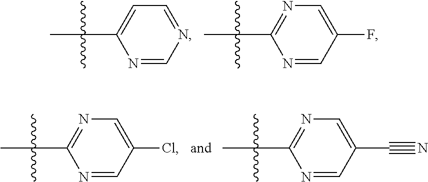

In another embodiment R₃ is a C₃-C₆cycloalkyl, preferably cyclopropyl or cyclobutyl. The C₃-C₆ cycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the cycloalkyl group.

In one preferred embodiment R₃ is

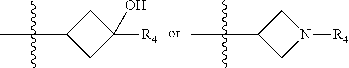

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl. In one preferred embodiment R₃ is

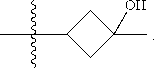 [SMILES: *C1CC(C)(O)C1]

In another preferred embodiment R₃ is

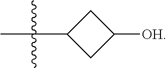 [SMILES: *C1CC(O)C1]

In another preferred embodiment R₃ is from C₁-C₃alkyl, preferably C₁-C₃alkyl, for example methyl, ethyl or propyl. The alkyl can be substituted with one or two substituents independently selected from the group consisting of —OH, halo, haloC₁-C₄ alkyl and C₁-C₄alkoxy. In one preferred embodiment R₃ is hydroxyC₁-C₆alkyl (i.e. C₁-C₆alkyl substituted with —OH), more preferably hydroxyC₁-C₃alkyl, preferable hydroxyethyl.

In one preferred embodiment R₃ is

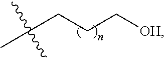

wherein n is 0, 1 or 2. In another preferred embodiment R₃ is

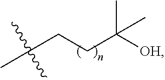

wherein n is 0, 1 or 2.

In another preferred embodiment there is provided a compound of formula (I) or (I′) having the formula (Ic)

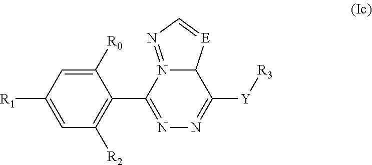

or a stereoisomer, a racemic mixture, a tautomer, a polymorph, a pharmaceutically acceptable salt, a prodrug, a hydrate, or a solvate thereof;

wherein

E is selected from N and CRₐ;

Rₐ is selected from the group consisting of —H, -C₁-C₃alkyl, —CF₃ and halo;

R₀ is selected from the group consisting of —H, C₁-C₃alkyl and halo;

R₁ is selected from the group consisting of —CF₃, OCF₃, —OCHF₂ and halo;

R₂ is selected from the group consisting of —OH, —H and —CF₃;

Y is selected from NH and O;

R₃ is selected from the group consisting of

4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄ alkyl, haloC₁-C₄alkyl, hydroxyC₁-C₄alkyl, —OH and halo;

5- or 6-membered aryl or heteroaryl, wherein heteroaryl contains one or two heteroatoms, wherein said heteroatom(s) is/are N, optionally substituted with one or two substituents independently selected from the group consisting of C₁-C₄ alkyl, hydroxyC₁-C₄alkyl, haloC₁-C₄alkyl, —CN and halo; C₃-C₆ cycloalkyl optionally substituted with one or two substituents independently selected from the group consisting of C₁-C₄ alkyl, haloC₁-C₄alkyl, hydroxyC₁-C₄ alkyl, —OH and halo; and

C₁-C₆alkyl substituted with one or two substituents independently selected from the group consisting of —OH, halo, haloC₁-C₄alkyl and C₁-C₄alkoxy.

In a preferred embodiment of the compound of formula (Ic) E is CRₐ, wherein Rₐ is as defined above. In one preferred embodiment of the compound of formula (Ic) E is CH. In another embodiment of the compound of formula (Ic) E is N.

In a preferred embodiment R₀ is selected from —H and C₁-C₃alkyl. In one preferred embodiment R₀ is —H. In another preferred embodiment R₀ is C₁-C₃alkyl, preferably methyl or ethyl, more preferably methyl.

In a preferred embodiment, R₁ is selected from —CF₃, OCF₃ and —OCHF₂. In one preferred embodiment R₁ is —CF₃. In another preferred embodiment R₁ is —OCF₃. In another preferred embodiment R₁ is —OCHF₂.

In another embodiment R₁ is halo. Where R₁ is halo, the halogen (halo) is preferably chloro.

In one preferred embodiment R₂ is —OH. In another embodiment R₂ is —H. In another embodiment R₂ is —CF₃.

In one preferred embodiment R₀ is —H, R₁ is selected from —CF₃, —OCF₃ and —OCHF₂, and R₂ is —OH or —H. In one preferred embodiment R₀ is —H, R₁ is —CF₃, and R₂ is —OH.

In preferred embodiments R₃ is independently selected from the group consisting of 4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄alkyl, haloC₁-C₄alkyl, hydroxyC₁-C₄ alkyl, —OH and halo;

6-membered aryl or heteroaryl, wherein heteroaryl contains one or two heteroatoms, wherein said heteroatom(s) is/are N, optionally substituted with one or two substituents independently selected from the group consisting of C₁-C₄alkyl, hydroxyC₁-C₄alkyl, haloC₁-C₄alkyl, —CN and halo;

C₃-C₆ cycloalkyl optionally substituted with one or two substituents independently selected from the group consisting of C₁-C₄alkyl, haloC₁-C₄alkyl, hydroxyC₁-C₄ alkyl, —OH and halo; and

C₁-C₆alkyl substituted with one or two substituents independently selected from the group consisting of —OH, halo, haloC₁-C₄ alkyl and C₁-C₄alkoxy.

In one preferred embodiment R₃ is selected from a 5- or 6-membered heterocycloalkyl containing one or two heteroatoms, preferably one heteroatom, wherein said heteroatom(s) is/are N. In one preferred embodiment R₃ is a 6-membered heterocycloalkyl containing one heteroatom, wherein said heteroatom is N. The 5- or 6-membered heterocycloalkyl can be substituted with one or two substituents independently selected from the group consisting of C₁-C₄alkyl, haloC₁-C₄alkyl, hydroxyC₁-C₄ alkyl, —OH and halo. The substituents can be at any available position on the heterocyclic group.

In one preferred embodiment R₃ is

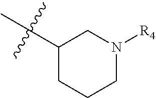

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In one preferred embodiment R₃ is

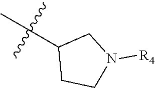

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In a further preferred embodiment, R₃ is

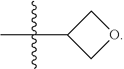 [SMILES: *C1COC1]

In another preferred embodiment R₃ is

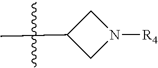

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl, whereby R₃ is

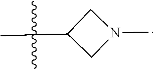 [SMILES: *C1CN(C)C1]

In another embodiment R₃ is selected from a 6-membered aryl or heteroaryl, wherein heteroaryl contains one or two heteroatoms, wherein said heteroatom(s) is/are N. The 6-membered aryl or heteroaryl can be substituted with one or two substituents independently selected from the group consisting of C₁-C₄alkyl, hydroxyC₁-C₄ alkyl, haloC₁-C₄ alkyl, —CN and halo. The substituents can be at any available position on the aryl or heteroaryl group.

In one embodiment R₃ is a 6-membered heteroaryl with two N heteroatoms. The 6-membered heteroaryl can be substituted with one or two substituents independently selected from the group consisting of C₁-C₄alkyl, hydroxyC₁-C₄alkyl, haloC₁-C₄ alkyl, —CN and halo. In one embodiment R₃ is selected from the group consisting of

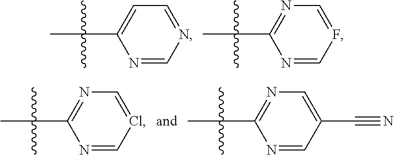

In another embodiment R₃ is a C₃-C₆ cycloalkyl, preferably cyclopropyl or cyclobutyl. The C₃-C₆ cycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the cycloalkyl group.

In one preferred embodiment R₃ is

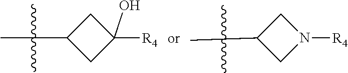

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl. In one preferred embodiment R₃ is

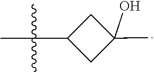 [SMILES: *C1CC(C)(O)C1]

In another preferred embodiment R₃ is

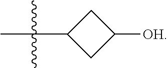 [SMILES: *C1CC(O)C1]

In another preferred embodiment R₃ is from C₁-C₆alkyl, preferably C₁-C₃alkyl, for example methyl, ethyl or propyl. The alkyl can be substituted with one or two substituents independently selected from the group consisting of —OH, halo, haloC₁-C₄alkyl and C₁-C₄alkoxy. In one preferred embodiment R₃ is hydroxyC₁-C₆alkyl (i.e. C₁-C₆ alkyl substituted with —OH), more preferably hydroxyC₁-C₃alkyl, preferable hydroxyethyl.

In one preferred embodiment R₃ is

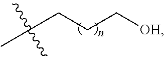

wherein n is 0, 1 or 2. In another preferred embodiment R₃ is

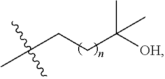

wherein n is 0, 1 or 2.

In another embodiment there is provided a compound of formula (I) having the formula (Id)

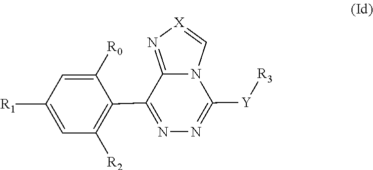

or a stereoisomer, a racemic mixture, a tautomer, a polymorph, a pharmaceutically acceptable salt, a prodrug, a hydrate, or a solvate thereof;

wherein Rₐ, R₀, R₁, R₂, R₃, X and Y are as defined in any of the above embodiments.

In a preferred embodiment of the compound of formula (Id) X is CRₐ, wherein Rₐ is as defined above.

In one preferred embodiment of the compound of formula (Ic) X is CH. In another embodiment of the compound of formula (Ic) X is N.

In a preferred embodiment R₀ is selected from —H and C₁-C₃alkyl. In one preferred embodiment R₀ is —H. In another preferred embodiment R₀ is C₁-C₃alkyl, preferably methyl or ethyl, more preferably methyl.

In a preferred embodiment, R₁ is selected from —CF₃, —OCF₃ and —OCHF₂. In one preferred embodiment R₁ is —CF₃. In another preferred embodiment R₁ is —OCF₃. In another preferred embodiment R₁ is —OCHF₂.

In another embodiment R₁ is halo. Where R₁ is halo, the halogen (halo) is preferably chloro.

In one preferred embodiment R₂ is —OH. In another embodiment R₂ is —H. In another embodiment R₂ is —CF₃.

In one preferred embodiment R₀ is —H, R₁ is selected from —CF₃, —OCF₃ and —OCHF₂, and R₂ is —OH or —H. In one preferred embodiment R₀ is —H, R₁ is —CF₃, and R₂ is —OH.

In preferred embodiments R₃ is independently selected from the group consisting of 4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄alkyl, hydroxyC₁-C₄ alkyl, —OH and halo;

6-membered aryl or heteroaryl, wherein heteroaryl contains one or two heteroatoms, wherein said heteroatom(s) is/are N, optionally substituted with one or two substituents independently selected from the group consisting of C₁-C₄ alkyl, hydroxyC₁-C₄ alkyl, haloC₁-C₄alkyl, —CN and halo;

C₃-C₆cycloalkyl optionally substituted with one or two substituents independently selected from the group consisting of C₁-C₄alkyl, haloC₁-C₄alkyl, hydroxyC₁-C₄alkyl, —OH and halo; and

C₁-C₆alkyl substituted with one or two substituents independently selected from the group consisting of —OH, halo, haloC₁-C₄alkyl and C₁-C₄alkoxy.

In one preferred embodiment R₃ is selected from a 5- or 6-membered heterocycloalkyl containing one or two heteroatoms, preferably one heteroatom, wherein said heteroatom(s) is/are N. In one preferred embodiment R₃ is a 6-membered heterocycloalkyl containing one heteroatom, wherein said heteroatom is N. The 5- or 6-membered heterocycloalkyl can be substituted with one or two substituents independently selected from the group consisting of C₁-C₄alkyl, haloC₁-C₄alkyl, hydroxyC₁-C₄alkyl, —OH and halo. The substituents can be at any available position on the heterocyclic group.

In one preferred embodiment R₃ is

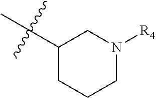

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In one preferred embodiment R₃ is

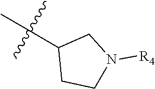

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In a further preferred embodiment, R₃ is

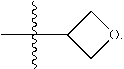 [SMILES: *C1COC1]

In another preferred embodiment R₃ is

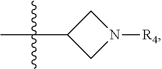

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl, whereby R₃ is

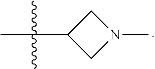 [SMILES: *C1CN(C)C1]

In another embodiment, R₃ is selected from a 6-membered aryl or heteroaryl, wherein heteroaryl contains one or two heteroatoms, wherein said heteroatom(s) is/are N. The 6-membered aryl or heteroaryl can be substituted with one or two substituents independently selected from the group consisting of C₁-C₄ alkyl, hydroxyC₁-C₄alkyl, haloC₁-C₄alkyl, —CN and halo. The substituents can be at any available position on the aryl or heteroaryl group.

In one embodiment, R₃ is a 6-membered heteroaryl with two N heteroatoms. The 6-membered heteroaryl can be substituted with one or two substituents independently selected from the group consisting of C₁-C₄alkyl, hydroxyC₁-C₄ alkyl, haloC₁-C₄ alkyl, —CN and halo. In one embodiment R₃ is selected from the group consisting of

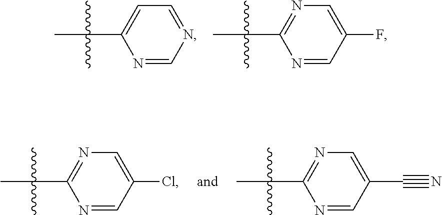 [SMILES: *c1ccncn1 ; *c1ncc(F)cn1 ; *c1ncc(Cl)cn1 ; *c1ncc(CN)cn1]

In another embodiment, R₃ is a C₃-C₆ cycloalkyl, preferably cyclopropyl or cyclobutyl. The C₃-C₆ cycloalkyl can be substituted with one or two substituents independently selected from the group consisting of C₁-C₄alkyl, haloC₁-C₄ alkyl, hydroxyC₁-C₄alkyl, —OH and halo. The substituents can be at any available position on the cycloalkyl group.

In one preferred embodiment, R₃ is

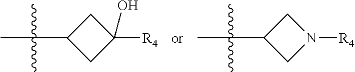

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl. In one preferred embodiment R₃ is

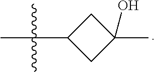 [SMILES: *C1CC(C)(O)C1]

In another preferred embodiment R₃ is

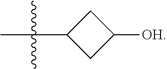 [SMILES: *C1CC(O)C1]

In another preferred embodiment R₃ is from C₁-C₆alkyl, preferably C₁-C₃alkyl, for example methyl, ethyl or propyl. The alkyl can be substituted with one or two substituents independently selected from the group consisting of —OH, halo, haloC₁-C₄ alkyl and C₁-C₄alkoxy. In one preferred embodiment R₃ is hydroxyC₁-C₆alkyl (i.e. C₁-C₆alkyl substituted with —OH), more preferably hydroxyC₁-C₃alkyl, preferable hydroxyethyl.

In one preferred embodiment R₃ is

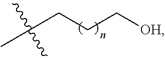

wherein n is 0, 1 or 2. In another preferred embodiment R₃ is

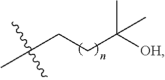

wherein n is 0, 1 or 2.

In another preferred embodiment there is provided a compound of formula (I) or (I′) having the formula (Ie)

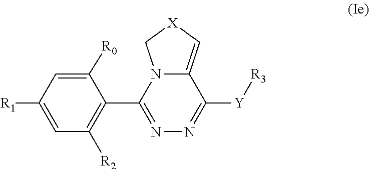

or a stereoisomer, a racemic mixture, a tautomer, a polymorph, a pharmaceutically acceptable salt, a prodrug, a hydrate, or a solvate thereof;

wherein

X is selected from N and CRₐ;

Rₐ is selected from the group consisting of -C₁-C₃alkyl, —CF₃ and halo;

R₀ is selected from the group consisting of —H, C₁-C₃alkyl and halo;

R₄ is selected from the group consisting of —CF₃, —OCF₃, —OCHF₂ and halo;

R₂ is selected from the group consisting of —OH, —H and —CF₃;

Y is selected from NH and O;

R₃ is selected from the group consisting of

4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄alkyl, haloC₁-C₄alkyl, hydroxyC₁-C₄alkyl, —OH and halo;

4-, 5- or 6-membered aryl or heteroaryl, wherein heteroaryl contains one or two heteroatoms, wherein said heteroatom(s) is/are N, optionally substituted with one or two substituents independently selected from the group consisting of C₁-C₄alkyl, hydroxyC₁-C₄ alkyl, haloC₁-C₄ alkyl, —CN and halo;

C₃-C₆cycloalkyl optionally substituted with one or two substituents independently selected from the group consisting of C₁-C₄ alkyl, haloC₁-C₄alkyl, hydroxyC₁-C₄alkyl, —OH and halo; and

C₁-C₆alkyl substituted with one or two substituents independently selected from the group consisting of —OH, halo, haloC₁-C₄ alkyl and C₁-C₄alkoxy.

In one preferred embodiment of the compound of formula (Ie) X is N. In another preferred embodiment of the compound of formula (Ie) X is CRₐ.

In a preferred embodiment R₀ is selected from —H and C₁-C₃alkyl. In one preferred embodiment R₀ is —H. In another preferred embodiment R₀ is C₁-C₃alkyl, preferably methyl or ethyl, more preferably methyl.

In a preferred embodiment, R₁ is selected from —CF₃, —OCF₃ and —OCHF₂. In one preferred embodiment R₁ is —CF₃. In another preferred embodiment R₁ is —OCF₃. In another preferred embodiment R₁ is —OCHF₂.

In another embodiment R₁ is halo. Where R₁ is halo, the halogen (halo) is preferably chloro.

In one preferred embodiment R₂ is —OH. In another embodiment R₂ is —H. In another embodiment R₂ is —CF₃.

In one preferred embodiment R₀ is —H, R₁ is selected from —CF₃, —OCF₃ and —OCHF₂, and R₂ is —OH or —H. In one preferred embodiment R₀ is —H, R₁ is —CF₃, and R₂ is —OH.

In preferred embodiments R₃ is independently selected from the group consisting of 4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄alkyl, haloC₁-C₄alkyl, hydroxyC₁-C₄alkyl, —OH and halo;

6-membered aryl or heteroaryl, wherein heteroaryl contains one or two heteroatoms, wherein said heteroatom(s) is/are N, optionally substituted with one or two substituents independently selected from the group consisting of C₁-C₄alkyl, hydroxyC₁-C₄ alkyl, haloC₁-C₄ alkyl, —CN and halo;

C₃-C₆cycloalkyl optionally substituted with one or two substituents independently selected from the group consisting of C₁-C₄alkyl, haloC₁-C₄alkyl, hydroxyC₁-C₄alkyl, —OH and halo; and

C₁-C₆ alkyl substituted with one or two substituents independently selected from the group consisting of —OH, halo, haloC₁-C₄ alkyl and C₁-C₄ alkoxy.

In one preferred embodiment R₃ is selected from a 5- or 6-membered heterocycloalkyl containing one or two heteroatoms, preferably one heteroatom, wherein said heteroatom(s) is/are N. In one preferred embodiment R₃ is a 6-membered heterocycloalkyl containing one heteroatom, wherein said heteroatom is N. The 5- or 6-membered heterocycloalkyl can be substituted with one or two substituents independently selected from the group consisting of C₁-C₄alkyl, haloC₁-C₄alkyl, hydroxyC₁-C₄ alkyl, —OH and halo. The substituents can be at any available position on the heterocyclic group.

In one preferred embodiment R₃ is

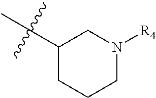

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In one preferred embodiment R₃ is

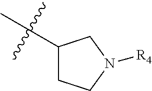

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl.

In a further preferred embodiment, R₃ is

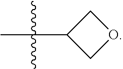 [SMILES: *C1COC1]

In another preferred embodiment R₃ is

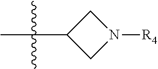

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl, whereby R₃ is

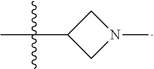 [SMILES: *C1CN(C)C1]

In another embodiment R₃ is selected from a 6-membered aryl or heteroaryl, wherein heteroaryl contains one or two heteroatoms, wherein said heteroatonn(s) is/are N. The 6-membered aryl or heteroaryl can be substituted with one or two substituents independently selected from the group consisting of C₁-C₄alkyl, hydroxyC₁-C₄ alkyl, haloC₁-C₄alkyl, —CN and halo. The substituents can be at any available position on the aryl or heteroaryl group.

In one embodiment R₃ is a 6-membered heteroaryl with two N heteroatoms. The 6-membered heteroaryl can be substituted with one or two substituents independently selected from the group consisting of C₁-C₄alkyl, hydroxyC₁-C₄ alkyl, haloC₁-C₄alkyl, —CN and halo. In one embodiment R₃ is selected from the group consisting of

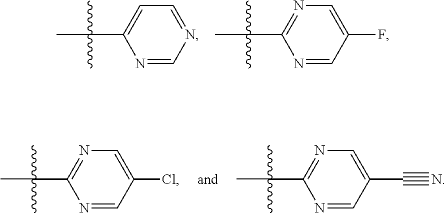

In another embodiment R₃ is a C₃-C₆ cycloalkyl, preferably cyclopropyl or cyclobutyl. The C₃-C₆cycloalkyl can be substituted with one or two substituents independently selected from the group consisting of C₁-C₄alkyl, haloC₁-C₄alkyl, hydroxyC₁-C₄ alkyl, —OH and halo. The substituents can be at any available position on the cycloalkyl group.

In one preferred embodiment R₃ is

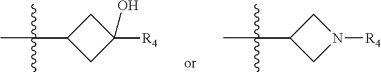

wherein R₄ is independently selected from —H or C₁-C₃alkyl. In a preferred embodiment R₄ is H. In another embodiment R₄ is C₁-C₃alkyl, for instance methyl, ethyl or propyl. In a preferred embodiment R₄ is methyl. In one preferred embodiment R₃ is

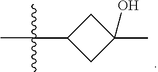 [SMILES: *C1CC(C)(O)C1]

In another preferred embodiment R₃ is

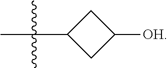 [SMILES: *C1CC(O)C1]

In another preferred embodiment R₃ is from C₁-C₆alkyl, preferably C₁-C₃alkyl, for example methyl, ethyl or propyl. The alkyl can be substituted with one or two substituents independently selected from the group consisting of —OH, halo, haloC₁-C₄alkyl and C₁-C₄alkoxy. In one preferred embodiment R₃ is hydroxyC₁-C₆alkyl (i.e. C₁-C₆alkyl substituted with —OH), more preferably hydroxyC₁-C₃alkyl, preferable hydroxyethyl.

In one preferred embodiment R₃ is

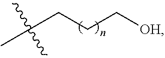

wherein n is 0, 1 or 2. In another preferred embodiment R₃ is

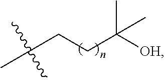

wherein n is 0, 1 or 2.

In a further embodiment, the present invention relates to the following compounds of formula (I′) or (I)

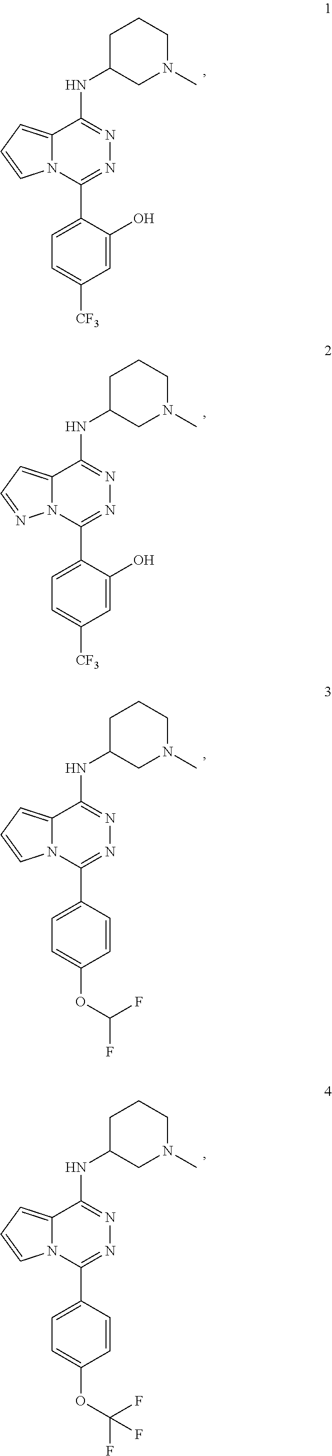 [SMILES: CN1CCCC(Nc2nnc(-c3ccc(C(F)(F)F)cc3O)n3cccc23)C1 ; CN1CCCC(Nc2nnc(-c3ccc(C(F)(F)F)cc3O)n3nccc23)C1 ; CN1CCCC(Nc2nnc(-c3ccc(OC(F)F)cc3)n3cccc23)C1 ; CN1CCCC(Nc2nnc(-c3ccc(OC(F)(F)F)cc3)n3cccc23)C1]

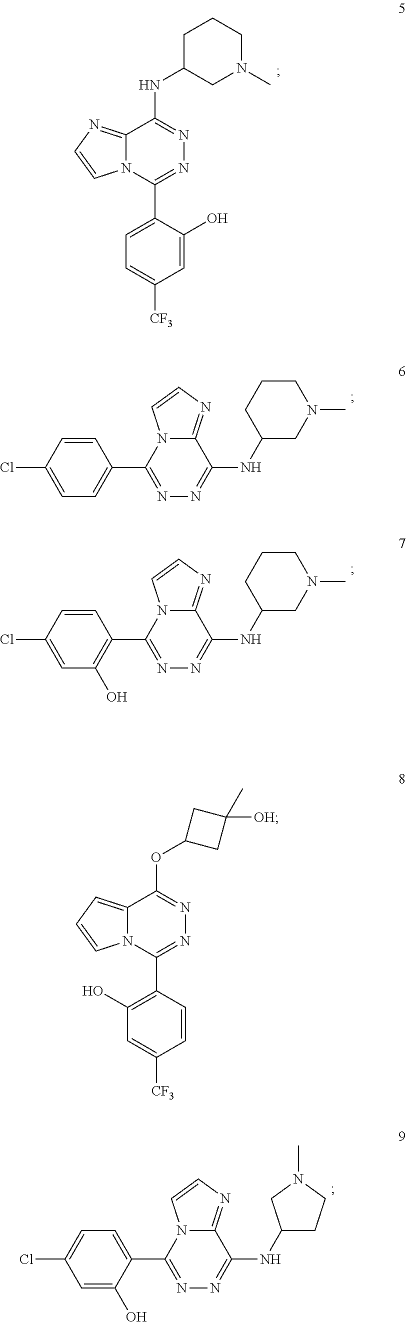 [SMILES: CN1CCCC(Nc2nnc(-c3ccc(C(F)(F)F)cc3O)n3ccnc23)C1 ; CN1CCCC(Nc2nnc(-c3ccc(Cl)cc3)n3ccnc23)C1 ; CN1CCCC(Nc2nnc(-c3ccc(Cl)cc3O)n3ccnc23)C1 ; CC1(O)CC(Oc2nnc(-c3ccc(C(F)(F)F)cc3O)n3cccc23)C1 ; CN1CCC(Nc2nnc(-c3ccc(Cl)cc3O)n3ccnc23)C1]

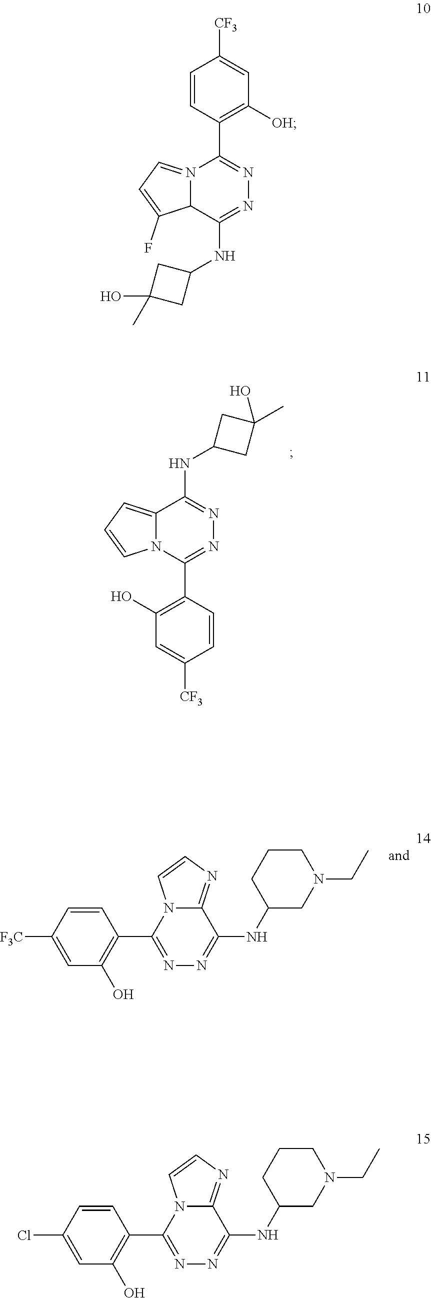

or a stereoisomer, a racemic mixture, a tautomer, a polymorph, a pharmaceutically acceptable salt, a prodrug, a hydrate, or a solvate thereof.

The present invention relates further to a pharmaceutical composition comprising a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, and optionally at least one pharmaceutically acceptable carrier, diluent, adjuvant or excipient.

In one embodiment, the pharmaceutical composition comprises a compound of formula (I′), (I), (Ia), (Pb), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, and optionally at least one pharmaceutically acceptable carrier, diluent, adjuvant or excipient.

Various embodiments of the invention are described herein, it will be recognized that features specified in each embodiment may be combined with other specified features to provide further embodiments of the present invention.

The present invention relates to a compound of formula (I′), (I), (Ia), (Pb), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for use as a medicament.

The present invention relates to a compound of formula (I′), (I), (Ia), (Pb), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a disease, disorder or abnormality which is responsive to the modulation, in particular inhibition of activation, of a component of the NLRP3 inflammasome pathway and/or which is responsive to the modulation, in particular decrease, of IL-1 beta and/or IL-18 levels. In one embodiment, the modulation is the reduction and/or inhibition of IL-1 beta and/or IL-1 beta levels. Particularly, the modulation is the reduction and/or inhibition of IL-1 beta.

In another embodiment, the present invention relates to a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for use in a method of reducing and/or inhibiting IL-1 beta. In particular, inhibiting IL-1 beta.

The present invention relates to a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a disease, disorder or abnormality which is responsive to the modulation, in particular inhibition of activation, of a component of the NLRP3 inflammasome pathway.

The present invention relates to a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a disease, disorder or abnormality which is responsive to the modulation, in particular inhibition of activation, of NLRP3 inflammasome pathway.

The present invention relates to a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a disease, disorder or abnormality which is responsive to the modulation, in particular decrease, of IL-1 beta and/or IL-18 levels.

In other words, the present invention relates to a method for treating, alleviating or preventing of a disease, disorder or abnormality which is responsive to the modulation, in particular inhibition of activation, of a component of the NLRP3 inflammasome pathway and/or which is responsive to the modulation, in particular decrease, of the IL-1 beta and/or IL-18 levels, wherein the method comprises administering a therapeutically effective amount of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, to a subject in need thereof (e.g. patient).

In one embodiment, the present invention relates to a method for treating, preventing or alleviating a disease, a disorder or abnormality which is responsive to the modulation, in particular inhibition of activation, of a component of the NLRP3 inflammasome pathway, wherein the method comprises administering a therapeutically effective amount of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, to a subject in need thereof (e.g. a patient).

The present invention further relates to a method for treating, preventing or alleviating a disease, a disorder or abnormality which is responsive to the modulation, in particular inhibition of activation, of NLRP3 inflammasome pathway, wherein the method comprises administering a therapeutically effective amount of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, to a subject in need thereof (e.g. a patient).

In one embodiment, the present invention relates to a method for treating, preventing or alleviating a disease, disorder or abnormality responsive to a modulation, in particular a decrease, of IL-1 beta and/or IL-18 levels, wherein the method comprises administering a therapeutically effective amount of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, to a patient in need thereof.

The present invention relates to the use of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for the manufacture of a medicament. In a further embodiment, the present invention relates to the use of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for the manufacture of a medicament for treating, alleviating or preventing a disease, disorder or abnormality which is responsive to the modulation, in particular inhibition of activation, of a component of the NLRP3 inflammasome pathway and/or which is responsive to the modulation, in particular decrease, of IL-1 beta and/or IL-18 levels. In one embodiment, the disease, disorder, or abnormality is selected from the list disclosed herein.

The present invention relates to the use of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for the manufacture of a medicament for treating, alleviating or preventing a disease, disorder or abnormality which is responsive to the modulation, in particular inhibition of activation, of a component of the NLRP3 inflammasome pathway.

The present invention relates to the use of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for the manufacture of a medicament for treating, alleviating or preventing a disease, disorder or abnormality which is responsive to the modulation, in particular inhibition of activation, of NLRP3 inflammasome pathway.

The present invention relates to the use of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for the manufacture of a medicament for treating, alleviating or preventing a disease, disorder or abnormality which is responsive to the modulation, in particular decrease, of IL-1 beta and/or IL-18 levels.

The present invention relates to the use of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for the manufacture of a medicament for reducing and/or inhibiting IL-1 beta and/or IL-1 beta levels. In one embodiment, the present invention relates to the use of a compound of the invention, as defined herein, for the manufacture of a medicament for reducing and/or inhibiting IL-1 beta. In another embodiment, the present invention relates to the use of a compound of the invention, as defined herein, for the manufacture of a medicament for reducing IL-1 beta.

In one embodiment, the present invention relates to a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a tauopathy by modulating a component of the inflammasome pathway, in particular, by modulating the NLRP3 inflammasome pathway.

In another embodiment, the disease, the disorder or the abnormality is responsive to modulation of one or more of IL-1β, IL-17, IL-18, IL-1 a, IL-37, IL-33 and Th17 cells, preferably: IL-1β and IL-18.

In yet another embodiment, the disease, disorder, or abnormality is a disease, disorder, or abnormality selected from Alzheimer's disease, Parkinson's disease, multiple sclerosis, cryopyrin-associated periodic syndromes (CAPS), non-alcoholic fatty liver disease (NAFLD), non-alcoholic steatohepatitis (NASH), and gout, such as Alzheimer's disease, Parkinson's disease, cryopyrin-associated periodic syndromes (CAPS), non-alcoholic fatty liver disease (NAFLD), non-alcoholic steatohepatitis (NASH), and gout.

In a further embodiment, the disease, disorder, or abnormality is a disease, a disorder or an abnormality of the immune system. In an embodiment, the disease, disorder, or abnormality is an inflammatory disease, disorder, or abnormality. In yet another embodiment, the disease, disorder, or abnormality is an autoimmune disease, disorder, or abnormality. In yet another embodiment, the disease, the disorder, or the abnormality is a disease, a disorder, or an abnormality of the central nervous system (CNS). In yet another embodiment, the disease, the disorder, or the abnormality can be a disease, disorder or abnormality or condition of the skin. The disease, the disorder or the abnormality can be a disease, disorder or abnormality or condition of the cardiovascular system. The disease, the disorder or the abnormality or condition can be a cancer, tumor or other malignancy. The disease, the disorder or the abnormality or condition can be a disease, disorder, or abnormality of the renal system. The disease, the disorder or the abnormality or condition can be a disease, disorder, or abnormality of the gastrointestinal tract. The disease, the disorder or the abnormality or condition can be a disease, disorder, or abnormality of the respiratory system. The disease, the disorder or the abnormality or condition can be a disease, disorder, or abnormality of the endocrine system. The disease, the disorder or the abnormality or condition can be liver related disease, disorder, or abnormality.

In one embodiment, the diseases, the disorders or the abnormalities which are responsive to the modulation, in particular inhibition of activation, of a component of the NLRP3 inflammasome pathway can be selected from Alzheimer's disease, Parkinson's disease, amyotrophic lateral sclerosis, demyelination, viral encephalitis, epilepsy, stroke, brain haemorrhage, atherosclerosis, asthma, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease, nonalcoholic fatty liver disease, nonalcoholic steatohepatitis, hypertension, myocardial infarction, oxalate-induced nephropathy, graft-versus host disease, type 1 diabetes, type 2 diabetes, rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, acne, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, acne, eczema, alopecia areata, actinic keratosis, hyperostosis, osteitis syndrome (SAPHO), multiple sclerosis (MS), psoriasis, Behcet's disease, Sjogren's syndrome, Schnitzler syndrome, chronic obstructive pulmonary disorder (COPD), steroid-resistant asthma, asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, chronic kidney disease, diabetic nephropathy, alcoholic liver disease, skin contact hypersensitivity, sunburn, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, hidradenitis suppurativa (HS), lung cancer metastasis, pancreatic cancers, gastric cancers, myelodisplastic syndrome, leukemia; polymyositis, colitis, helminth infection, bacterial infection, abdominal aortic aneurism, wound healing, depression, psychological stress, pericarditis including Dressler's syndrome, ischaemia reperfusion injury, frontotemporal dementia, HIV-associated neurocognitive disorder, Coronavirus-associated inflammatory pathologies, traumatic brain and spinal cord injury, inflammatory pain, chronic pain, neuropathic pain, metastatic cancer-induced bone pain, chemotherapy induced peripheral neuropathy, migraine, and amyloidosis (including AL amyloidosis, AA amyloidosis, ATTR amyloidosis, hereditary amyloidoses (including apolipoprotein A-I (AApoAI), apolipoprotein A-II (AApoAll), gelsolin (AGeI), fibrinogen (AFib), and lysozyme (ALys)), Beta-2 Microglobulin amyloidosis, iAPP amyloidosis).

In one embodiment, the diseases, the disorders or the abnormalities which are responsive to the modulation, in particular inhibition of activation, of a component of the NLRP3 inflammasome pathway can be selected from Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease, nonalcoholic fatty liver disease, nonalcoholic steatohepatitis, hypertension, myocardial infarction, oxalate-induced nephropathy, graft-versus host disease, type 1 diabetes, type 2 diabetes, rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, acne, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, acne, eczema, alopecia areata, actinic keratosis, hyperostosis, osteitis syndrome (SAPHO), multiple sclerosis (MS), psoriasis, Behcet's disease, Sjogren's syndrome, Schnitzler syndrome, chronic obstructive pulmonary disorder (COPD), steroid-resistant asthma, asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, chronic kidney disease, diabetic nephropathy, alcoholic liver disease, skin contact hypersensitivity, sunburn, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, hidradenitis suppurativa (HS), lung cancer metastasis, pancreatic cancers, gastric cancers, myelodisplastic syndrome, leukemia; polymyositis, colitis, helminth infection, bacterial infection, abdominal aortic aneurism, wound healing, depression, psychological stress, pericarditis including Dressler's syndrome, ischaemia reperfusion injury, frontotemporal dementia, HIV-associated neurocognitive disorder, Coronavirus-associated inflammatory pathologies, and traumatic brain injury.

Preferably, the diseases, the disorders or the abnormalities are selected from Alzheimer's disease, Parkinson's disease, amyotrophic lateral sclerosis, multiple sclerosis, demyelination, viral encephalitis, epilepsy, stroke, chronic pain, atherosclerosis, asthma and allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, and Hereditary Recurrent Fevers (HRFs).

In one embodiment, the diseases, the disorders or the abnormalities are selected from Alzheimer's disease, Parkinson's disease, amyotrophic lateral sclerosis, demyelination, viral encephalitis, epilepsy, stroke, atherosclerosis, asthma and allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, and Hereditary Recurrent Fevers (HRFs).

More preferably, the diseases, the disorders or the abnormalities are selected from Alzheimer's disease, Parkinson's disease, amyotrophic lateral sclerosis, multiple sclerosis, demyelination, viral encephalitis, epilepsy, stroke, atherosclerosis, asthma and allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 and type 2 diabetes, rheumatoid arthritis, and myelodysplastic syndrome.

In one embodiment, the diseases, the disorders or the abnormalities are selected from Alzheimer's disease, Parkinson's disease, amyotrophic lateral sclerosis, demyelination, viral encephalitis, epilepsy, stroke, atherosclerosis, asthma and allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 and type 2 diabetes, rheumatoid arthritis, and myelodysplastic syndrome.

Even more preferably, the diseases, the disorders or the abnormalities are selected from Alzheimer's disease, Parkinson's disease, multiple sclerosis, cryopyrin-associated periodic syndromes (CAPS), non-alcoholic fatty liver disease (NAFLD), non-alcoholic steatohepatitis (NASH), rheumatoid arthritis and gout. Even more preferably, the diseases, the disorders or the abnormalities are selected from Alzheimer's disease, Parkinson's disease, multiple sclerosis, cryopyrin-associated periodic syndromes (CAPS), rheumatoid arthritis and gout.

In one embodiment, the diseases, the disorders or the abnormalities are selected from Alzheimer's disease, Parkinson's disease, cryopyrin-associated periodic syndromes (CAPS), non-alcoholic fatty liver disease (NAFLD), non-alcoholic steatohepatitis (NASH), rheumatoid arthritis and gout; such as Alzheimer's disease, Parkinson's disease, cryopyrin-associated periodic syndromes (CAPS), rheumatoid arthritis and gout.

In one embodiment, the present invention relates to a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for use in the treatment, alleviation or prevention of a IL-18 and/or IL-1 beta related disease by modulating a component of the NLRP3 inflammasome pathway, in particular, by modulating NLRP3 inflammasome pathway. The IL-18 and/or IL-1 beta levels in a subject are decreased as a result of the administration of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof.

IL-18 and/or IL-1 beta related diseases, disorders or abnormalities are selected from chronic obstructive pulmonary disease (COPD), transfusion-related lung injury, bronchopulmonary dysplasia (BPD), acute respiratory distress syndrome (ARDS), pediatric autoinflammatory disease or condition, Still's disease, particularly Adult Still's disease or juvenile Still's disease, juvenile rheumatoid arthritis (JRA), juvenile idiopathic arthritis (JIA), systemic juvenile onset idiopathic arthritis (SoJIA), systemic juvenile idiopathic arthritis (sJIA), interstitial lung disease (ILD), macrophage activation syndrome (MAS) including primary, secondary and recurrent MAS, hemophagocytic lymphohistiocytosis (HLH), Familial (hereditary) hemophagocytic lymphohistiocytosis (FHLH) associated with gene defects in perforin, munc 13-4 and 18-2, synthaxin 11, immune deficiencies such as Chediak-Higashi syndrome (CHS), Griscelli syndrome (GS), X-linked lymphoproliferative syndrome (XLP2), X-linked inhibitor of apoptosis protein deficiency (XIAP), acquired hemophagocytic lymphohistiocytosis associated with infectious conditions especially Herpes virus such as EBV and other pathogens, autoinflammatory syndrome associated with NLRC4 mutations, Giant Cell Arteritis (GCA), acne, pyogenic arthritis pyoderma gangrenosum and acne (PAPA), pulmonary sarcoidosis, heart failure, ischemic heart disease, dry eye disease (DED), keratitis, corneal ulcer and abrasion, iritis, glaucoma, Sjogren's syndrome, autoimmune uveitis, Behcet's disease, conjunctivitis, allergic conjunctivitis, diabetes type 2, solid organ and hematologic stem cell transplantation, ischemia reperfusion injury, familial Mediterranean fever (FMF), tumor necrosis factor receptor 1-associated periodic syndromes (TRAPS), hyper-IgD syndromes (mevalonate kinase gene mutation), gout, Schnitzler syndrome, Wegener's granulomatosis also called granulomatosis with polyangitis (GPA), Hashimoto's thyroiditis, Crohn's disease, early onset inflammatory bowel disease (EOIBD), very EOIBD (VEOIBD), infantile IBD, neonatal IBD, ulcerative colitis and Blau syndrome (NOD-2 mutation).

The modulation of NLRP3 inflammasome pathway appears to be beneficial in diseases or disorders or abnormalities with altered IL-18 levels and/or IL-1 beta, which lead to pathological inflammation.

The present invention relates to compound of formula (I′), (I), (Ia), (Ib), (Ic), (Id) or (Ie), as defined in the present invention that are modulators of NLRP3 inflammasome activity and/or modulators of IL-18 and/or IL-1b levels in a subject.

In one embodiment, the invention provides a pharmaceutical composition comprising a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, and at least one further biologically active compound. Optionally, the pharmaceutical combination may comprise a pharmaceutically acceptable carrier, diluent, adjuvant or excipient as described herein.

In another embodiment, the present invention relates to a pharmaceutical composition comprising a combination of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, and at least one further biologically active compound differing from the compound of formula (I′), (I), (Ia), (Ib), (Ic), (Id) or (Ie), and optionally comprising at least one pharmaceutically acceptable carrier, diluent, adjuvant or excipient.

In another embodiment, the present invention relates to a pharmaceutical composition comprising a combination of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, and at least one further biologically active compound differing from the compound of formula (I′), (I), (Ia), (Ib), (Ic), (Id) or (Ie), and optionally comprising at least one pharmaceutically acceptable carrier, diluent, adjuvant or excipient.

In particular the further biologically active compound can be one used for the treatment of a disease, disorder or abnormality which targets a different pathomechanism, e.g. an anti-amyloid beta antibody, anti-Tau antibody, amyloid beta small molecule inhibitor, Tau aggregation small molecule inhibitor, anti-alpha synuclein antibody or alpha-synuclein aggregation small molecule inhibitor, anti-TDP-43 antibody or anti-TDP-43 aggregation small molecule inhibitor, among others. When a compound of the invention is used in combination with a further biologically active compound, the dose of each compound may differ from the dose if the compound is used as a monotherapy. Such biologically active compounds are well known from the literature. Such biological active compound is, for example, a chemical compound, peptide, antibody, antibody fragment, or nucleic acid, which is therapeutically active or enhances the therapeutic activity when administered to a subject (e.g., patient) in combination with a compound of the invention.

In another embodiment, the present invention relates to a pharmaceutical composition comprising a combination comprising a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, and at least one further biologically active compound differing from the compound of formula (I′), (I), (Ia), (Ib), (Ic), (Id) or (Ie), and optionally comprising at least one pharmaceutically acceptable carrier, diluent, adjuvant or excipient, for use as a medicament.

The term “combination” refers to either a fixed combination in one dosage unit form, or a combined administration where a compound of the present invention and a combination partner (e.g. another drug as explained above, also referred to as “therapeutic agent” or “further biologically active compound”) may be administered independently at the same time or separately within time intervals.

In another embodiment, the present invention relates to combination, in particular a pharmaceutical combination, comprising a therapeutically effective amount of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, and at least one further biologically active compound, and optionally at least one pharmaceutically acceptable carrier, diluent, adjuvant or excipient. In particular, the at least one further biologically active compound is a compound differing from a compound of formula (I′), (I), (Ia), (Ib), (Ic), (Id) or (Ie).

In another embodiment, the present invention relates to a combination comprising a therapeutically effective amount of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, and at least one further biologically active compound differing from the compound of formula (I′), (I), (Ia), (Ib), (Ic), (Id) or (Ie), and optionally comprising at least one pharmaceutically acceptable carrier, diluent, adjuvant or excipient, for use as a medicament.

The present invention relates to the use of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, as an analytical reference or an in vitro screening tool. The compounds of the present invention, or a stereoisomer, a racemic mixture, a tautomer, a polymorph, a pharmaceutically acceptable salt, a prodrug, a hydrate, or a solvate thereof, can be used as an analytical reference or an in vitro screening tool for characterization of cells with activated NLRP3 inflammasome pathway and for testing of compounds targeting the NLRP3 inflammasome pathway.

Accordingly, the invention provides the use of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for treating, alleviating or preventing a disorder or an abnormality which is responsive to the modulation of a component of the NLRP3 inflammasome pathway or which is responsive to the modulation, in particular decrease, of IL-1 beta and/or IL-18 levels, wherein the medicament is prepared for administration with further biologically active agent. The invention also provides the use of further biologically active agent for treating alleviating or preventing a disorder or an abnormality which is responsive to the modulation of a component of the NLRP3 inflammasome pathway or which is responsive to the modulation, in particular decrease, of IL-1 beta and/or IL-18 levels, wherein the further biologically active agent is administered with a compound of the present invention, or a stereoisomer, a racemic mixture, a tautomer, a polymorph, a pharmaceutically acceptable salt, a prodrug, a hydrate, or a solvate thereof.

In another embodiment, the invention provides the use of a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for treating, alleviating or preventing a disorder or an abnormality which is responsive to the modulation of a component of the NLRP3 inflammasome pathway or which is responsive to the modulation, in particular decrease, of IL-1 beta and/or IL-18 levels, wherein the modulation is the reduction and/or the inhibition of IL-1 beta and/or IL-1 beta levels. Preferably, the modulation is the reduction and/or the inhibition of IL-1 beta. Preferably, the modulation is the inhibition of IL-1 beta. In another embodiment, the invention provides a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for use as a medicament, in particular for inhibiting IL-1 beta.

In another embodiment, the invention also provides a compound of formula (I′), (I), (Ia), (Ib), (Ic), (Id) or (Ie), as defined in the present invention, or a stereoisomer, a racemic mixture, a tautomer, a polymorph, a pharmaceutically acceptable salt, a prodrug, a hydrate, or a solvate thereof, for use in a method of treating, alleviating or preventing a disease, disorder or abnormality which is responsive to the modulation of a component of the NLRP3 inflammasome pathway or which is responsive to the modulation, in particular decrease, of IL-1 beta and/or IL-18 levels, wherein said compound of formula (I′), (I), (Ia), (Ib), (Ic), (Id) or (Ie), is prepared for administration with further biologically active compound (as defined herein).

In another embodiment, the present invention also provides a method of treating alleviating or preventing a disease, disorder or abnormality which is responsive to the modulation of a component of the NLRP3 inflammasome pathway or which is responsive to the modulation, in particular decrease, of IL-1 beta and/or IL-18 levels, selected from Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease, nonalcoholic fatty liver disease, nonalcoholic steatohepatitis, hypertension, myocardial infarction, oxalate-induced nephropathy, graft-versus host disease, type 1 diabetes, type 2 diabetes, rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, acne, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, acne, eczema, alopecia areata, actinic keratosis, hyperostosis, osteitis syndrome (SAPHO), multiple sclerosis (MS), psoriasis, Behcet's disease, Sjogren's syndrome, Schnitzler syndrome, chronic obstructive pulmonary disorder (COPD), steroid-resistant asthma, asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, chronic kidney disease, diabetic nephropathy, alcoholic liver disease, skin contact hypersensitivity, sunburn, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, hidradenitis suppurativa (HS), lung cancer metastasis, pancreatic cancers, gastric cancers, myelodisplastic syndrome, leukemia; polymyositis, colitis, helminth infection, bacterial infection, abdominal aortic aneurism, wound healing, depression, psychological stress, pericarditis including Dressler's syndrome, ischaemia reperfusion injury, frontotemporal dementia, HIV-associated neurocognitive disorder, Coronavirus-associated inflammatory pathologies, and traumatic brain injury; preferably the disorder is selected from Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, Hereditary Recurrent Fevers (HRFs), and amyloidosis (including AL amyloidosis, AA amyloidosis, ATTR amyloidosis, hereditary amyloidoses (including apolipoprotein A-I (AApoAI), apolipoprotein A-II (AApoAll), gelsolin (AGeI), fibrinogen (AFib), and lysozyme (ALys)), Beta-2 Microglobulin amyloidosis, iAPP amyloidosis), comprising administering to the subject a therapeutically effective amount of a compound of formula (I′), (I), (Ia), (Ib), (Ic), (Id) or (Ie), as defined herein, or stereoisomers, or racemic mixtures, or tautomers, or polymorph, or pharmaceutically acceptable salts, or hydrates, or solvates thereof.

In another embodiment, the present invention also provides a method of inhibiting IL-1 beta in a subject in need, the method comprising administering to a subject in need thereof a therapeutically effective amount of a compound of formula (I′), (I), (Ia), (Ib), (Ic), (Id) or (Ie), or a stereoisomer, a racemic mixture, a tautomer, a polymorph, a pharmaceutically acceptable salt, a prodrug, a hydrate, or a solvate thereof.

In particular, the disease, disorder or abnormality is one which is responsive to the inhibition of activation of the NLRP3 inflammasome pathway. More particularly, the disease, disorder or abnormality is responsive to the modulation of one or more of, for example, but not limited to, IL-1β or IL-18. For example, the disease, disorder, or abnormality is responsive to the modulation of one or more of IL-1β, IL-17, IL-18, IL-1 a, IL-37, IL-33 and Th17 cells, preferably the disease, disorder, or abnormality is responsive to the modulation of IL-1β and/or IL-18.

Any combination of the embodiments, preferred embodiments and more preferred embodiments disclosed herein is also envisaged in the present invention.

Pharmaceutical Compositions

While it is possible for the compounds of the present invention, or a stereoisomer, a racemic mixture, a tautomer, a polymorph, a pharmaceutically acceptable salt, a prodrug, a hydrate, or a solvate thereof, to be administered alone, it is preferable to formulate them into a pharmaceutical composition in accordance with standard pharmaceutical practice. Thus, the invention also provides a pharmaceutical composition which comprises a therapeutically effective amount of a compound of formula (I′), (I), (Ia), (Ib), (Ic), (Id) or (Ie) or a stereoisomer, a racemic mixture, a tautomer, a polymorph, a pharmaceutically acceptable salt, a prodrug, a hydrate, or a solvate thereof, optionally in admixture with a pharmaceutically acceptable carrier, diluent, adjuvant or excipient.

The term “a therapeutically effective amount” of a compound of the present invention refers to an amount of the compound of the present invention (i.e. a compound of formula (I′), (I), (Ia), (Ib), (Ic), (Id) or (Ie), or a stereoisomer, a racemic mixture, a tautomer, a polymorph, a pharmaceutically acceptable salt, a prodrug, a hydrate, or a solvate thereof) that will elicit the biological or medical response of a subject, for example, reduction or inhibition of an enzyme or a protein activity, or ameliorate symptoms, alleviate conditions, slow or delay disease progression, or prevent a disease, a disorder or an abnormality, etc. In one embodiment, the term “a therapeutically effective amount” refers to the amount of the compound of the present invention that, when administered to a subject in need thereof (e.g. a patient), is effective to at least partially alleviate, prevent and/or ameliorate a disease, a disorder, or an abnormality which is responsive to the modulation of a component of the NLRP3 inflammasome pathway or which is responsive to the modulation, in particular decrease, of IL-1 beta and/or IL-18.

Pharmaceutically acceptable carriers, diluents, adjuvants and excipients are well known in the pharmaceutical art and are described, for example, in Remington's Pharmaceutical Sciences, 18th Ed. (Alfonso R. Gennaro, ed.; Mack Publishing Company, Easton, P A, 1990); Remington: the Science and Practice of Pharmacy 19^{th} Ed. (Lippincott, Williams & Wilkins, 1995); Handbook of Pharmaceutical Excipients, 3^{rd} Ed. (Arthur H. Kibbe, ed.; Amer. Pharmaceutical Assoc, 1999); Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); Fiedler's “Lexikon der Hilfsstoffe” 5^{th} Ed., Edition Cantor Verlag Aulendorf 2002; “The Handbook of Pharmaceutical Excipients”, 4^{th} Ed., American Pharmaceuticals Association, 2003; and Goodman and Gilman's: the Pharmacological Basis of Therapeutics (Louis S. Goodman and Lee E. Limbird, eds.; McGraw Hill, 1992), the disclosures of which are hereby incorporated by reference.

The carriers, diluents, adjuvants and pharmaceutical excipients can be selected with regard to the intended route of administration and standard pharmaceutical practice. These compounds must be acceptable in the sense of being not deleterious to the recipient thereof.

Pharmaceutically useful excipients that may be used in the formulation of the pharmaceutical composition of the present invention may comprise, for example, vehicles, solvents (such as monohydric alcohols such as ethanol, isopropanol and polyhydric alcohols such as glycols), edible oils (such as soybean oil, coconut oil, olive oil, safflower oil, and cottonseed oil), oily esters (such as ethyl oleate and isopropyl myristate), binders (such as hydroxypropylmethyl cellulose (HPMC), hydroxypropyl cellulose (HPC), pregelatinized starch and combinations thereof), solubilizers, thickening agents, stabilizers, disintegrants (such as carboxymethylcellulose calcium (CMC-Ca), carboxymethylcellulose sodium (CMC-Na), crosslinked PVP (e.g., crospovidone, Polyplasdone® or Kollidon® XL), alginic acid, sodium alginate, guar gum, cross-linked CMC (croscarmellose sodium, e.g. Ac-Di-Sole), carboxymethyl starch-Na (sodium starch glycolate) (e.g., Primojel® or Explotab®), preferably crosslinked PVP and/or croscarmellose sodium), glidants (such as colloidal SiO₂ (e.g., Aerosil® 200), magnesium trisilicate, powdered cellulose, talc and combinations thereof), lubricating agents (such as magnesium stearate, aluminium or calcium silicate, stearic acid, hydrogenated castor oil, talc, glyceryl behenate, sodium stearate fumarate and combinations thereof), buffering agents, emulsifiers, wetting agents, suspending agents, sweetening agents, colorants, flavors, coating agents, preservatives, antioxidants, processing agents, drug delivery modifiers and enhancers (such as calcium phosphate), magnesium stearate, talc, monosaccharides, disaccharides, starch, gelatine, cellulose, methylcellulose, sodium carboxymethyl cellulose, dextrose, hydroxypropyl-β-cyclodextrin, polyvinylpyrrolidone, low melting waxes, and ion exchange resins.

The carrier is not particularly limited and will depend on the route of administration as well as the form of the pharmaceutical composition (i.e., solid, liquid, etc.). Suitable carriers include, without limitation, polyols such as mannitol, sorbitol, xylitol; disaccharides such as lactose, sucrose, dextrose and maltose; polysaccharides such as maltodextrin and dextran; starches such as corn starch; celluloses such as microcrystalline cellulose, sodium carboxy methylcellulose, low-substituted hydroxypropyl cellulose, hydroxyl ethyl cellulose, hydroxypropyl cellulose or mixtures thereof; cyclodextrins and inorganic agents such as dicalcium phosphate, calcium hydrogen phosphate; hydroxyapatite, tricalcium phosphate, talcum and silica. Microcrystalline cellulose, sucrose and/or lactose are preferred as carriers. Combinations thereof can also be employed. Carriers can include also protein and cell penetrating peptides which should be selected depending on the route of administration and target.

The diluent is not particularly limited and will depend on the route of administration as well as the form of the pharmaceutical composition (i.e., solid, liquid, etc.). Diluents include, for instance, water, ethanol, propylene glycol and glycerin, and combinations thereof.

An adjuvant is an additive which has few or no pharmacological effects by themselves, but that increases the efficacy or potency of the compounds of the invention if they are administered together.

The routes for administration (delivery) of the compounds of the invention include, but are not limited to, one or more of the following routes of administration: oral (e.g., as a tablet, capsule, or as an ingestible solution), topical, mucosal (e.g. as a nasal spray or aerosol for inhalation), nasal, parenteral (e.g., by an injectable form), gastrointestinal, intraspinal, intraperitoneal, intramuscular, intravenous, intraarterial, intrathecal, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual.

For example, the compounds can be administered orally in the form of tablets, capsules, ovules, elixirs, solutions or suspensions, which may contain flavoring or coloring agents, for immediate-, delayed-, modified-, sustained-, pulsed- or controlled-release applications.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatine and acacia. Additionally, lubricating agents such as magnesium stearate, stearic acid, glyceryl behenate and talc may be included. Solid compositions of a similar type may also be employed as fillers in gelatine capsules. Preferred excipients in this regard include starch, cellulose, milk sugar e.g. lactose or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

If the compounds of the present invention, as disclosed herein, are administered parenterally, then examples of such administration include one or more of: intravenously, intraarterially, intraperitoneally, intrathecally, intraventricularly, intraurethrally, intrasternally, intracranially, intramuscularly or subcutaneously administering the compounds; and/or by using infusion techniques. For parenteral administration, the compounds can be used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

As indicated, the compounds of the present invention can be administered intranasally or by inhalation and are conveniently delivered in the form of a dry powder inhaler or an aerosol spray presentation from a pressurized container, pump, spray or nebulizer with the use of a suitable propellant, e.g. dichlorodifluoromethane, trichlorofluoromethane, dichlorotetrafluoroethane, a hydro-fluoroalkane such as 1,1,1,2-tetrafluoroethane (HFA134AT) or 1,1,1,2,3,3,3-heptafluoropropane (HFA 227EA), carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g. using a mixture of ethanol and the propellant as the solvent, which may additionally contain a lubricant, e.g. sorbitan trioleate. Capsules and cartridges (made, for example, from gelatine) for use in an inhaler or insufflator may be formulated to contain a powder mix of the compound and a suitable powder base such as lactose or starch.

Alternatively, the compounds of the present invention, as defined herein, can be administered in the form of a suppository or pessary, or it may be applied topically in the form of a gel, hydrogel, lotion, solution, cream, ointment or dusting powder. The compounds of the present invention, as defined herein, may also be dermally or transdermally administered, for example, by the use of a skin patch.

They may also be administered by the pulmonary or rectal routes. They may also be administered by the ocular route. For ophthalmic use, the compounds can be formulated as micronized suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the compounds of the present invention can be formulated as a suitable ointment containing the active compound suspended or dissolved in, for example, a mixture with one or more of the following: mineral oil, liquid petrolatum, white petrolatum, propylene glycol, emulsifying wax and water. Alternatively, they can be formulated as a suitable lotion or cream, suspended or dissolved in, for example, a mixture of one or more of the following: mineral oil, sorbitan monostearate, polyethylene glycol, liquid paraffin, polysorbate 60, cetyl ester wax, cetearyl alcohol, 2-octyldodecanol, benzyl alcohol and water.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

The claimed compounds, as defined herein, can be used for the treatment, alleviation or prevention of the recited conditions alone or in combination with one or more other biologically active compounds, as defined herein. In particular, the other biologically active compound can be one used for the treatment, alleviation, or prevention of the recited diseases.

The combinations referred to above may conveniently be presented for use in the form of a pharmaceutical formulation. The individual components of such combinations may be administered either sequentially or simultaneously in separate or combined pharmaceutical formulations by any convenient route. When administration is sequential, either the compound of the invention or the other biologically active compound may be administered first. When administration is simultaneous, the combination may be administered either in the same or different pharmaceutical composition. When combined in the same formulation it will be appreciated that the two compounds must be stable and compatible with each other and the other components of the formulation. When formulated separately they may be provided in any convenient formulation, conveniently in such manners as are known for such compounds in the art.

The pharmaceutical compositions of the invention can be produced in a manner known per se to the skilled person as described, for example, in Remington's Pharmaceutical Sciences, 15th Ed., Mack Publishing Co., New Jersey (1975).

The compounds according to the present invention, as disclosed herein, can also be provided in the form of a mixture with at least one further biologically active compound and/or a pharmaceutically acceptable carrier, diluent, adjuvant, or excipient. The compound and/or the further biologically active compound are preferably present in a therapeutically effective amount.

The nature of the further biologically active compound will depend on the intended use of the mixture. The further biologically active substance or compound may exert its biological effect by the same or a similar mechanism as the compound according to the invention or by an unrelated mechanism of action or by a multiplicity of related and/or unrelated mechanisms of action.

The invention also includes all suitable isotopic variations of the compounds of the invention. An isotopic variation of the compound of the invention is defined as one in which at least one atom is replaced by an atom having the same atomic number but an atomic mass different from the atomic mass usually found in nature. Examples of isotopes that can be incorporated into compounds of the invention include isotopes of hydrogen, carbon, nitrogen, oxygen, sulphur, fluorine and chlorine such as ²H, ³H, ¹³C, ¹⁴C, ¹⁵N, ¹⁷O, ¹⁸O, ³⁵S, ¹⁸F and ³⁶Cl respectively. Certain isotopic variations of the invention, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and delectability. ¹⁸F-labeled compounds are particularly suitable for imaging applications such as PET. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the compounds of the invention can generally be prepared by conventional procedures such as by the illustrative methods or by the preparations described in the Examples and Preparations hereafter using appropriate isotopic variations of suitable reagents.

Methods of Use of the Invention

There is evidence for a role of NLRP3-induced IL-1 and IL-18 in the inflammatory responses occurring in connection with, or as a result of, a multitude of different diseases, disorders or abnormalities which is responsive to the modulation of a component of the NLRP3 inflammasome pathway and/or which is responsive to the modulation of IL-1 beta and/or IL-18 levels. (Menu et al., Clinical and Experimental Immunology, 2011, 166, 1-15; Strowig et al., Nature, 2012, 481, 278-286).

The invention provides a compound of formula (I′), (I), (Ia), (Ib), (Ic), (Id) or (Ie), as defined herein, or a stereoisomer, a racemic mixture, a tautomer, a polymorph, a pharmaceutically acceptable salt, a prodrug, a hydrate, or a solvate thereof, which exhibits valuable pharmacological properties, e.g. NRLP3 inhibiting properties on the NLRP3 inflammasome pathway. Said compounds of the invention may be useful in the treatment, alleviation or prevention of a disease, or a disorder or an abnormality which is responsive to the modulation of a component of the NLRP3 inflammasome pathway and/or which is responsive to the modulation of IL-1 beta and/or IL-18 levels. A number of diseases, disorders or abnormalities have been shown to be involve in NLRP3 including, for example, one of the following:
A. Central nervous system disease (CNS), disorder, or abnormality, such as Alzheimer's disease, Parkinson's disease, dementia, frontotemporal dementia, Huntington's disease, cerebral malaria, brain injury from pneumococcal meningitis, motor neuron disease, traumatic brain injury, amyotrophic lateral sclerosis, or multiple sclerosis (MS);
B. Immune disease, disorder, or abnormality (e.g. autoimmune disease, disorder or abnormality, and disease, disorder, or abnormality, involving the immune system), such as type 1 diabetes, hidradenitis suppurativa (HS), Schnitzler syndrome, multiple sclerosis (MS) including primary progressive multiple sclerosis (PPMS), Sjogren's syndrome, secondary progressive multiple sclerosis (SPMS), TNF receptor associated periodic syndrome (TRAPS), graft-versus host disease or relapsing remitting multiple sclerosis (RRMS);
C. Inflammatory disease, including auto-inflammation and inflammation occurring as a result of an inflammatory disease, disorder, or abnormality, such as mevalonate kinase deficiency (MKD), hyperimmunoglobulinemia D, cryopyrin-associated periodic syndromes (CAPS), Muckle-Wells syndrome (MWS), familial cold autoinflammatory syndrome (FCAS), neonatal-onset multisystem inflammatory disease (NOMID), familial Mediterranean fever (FMF), acne, pyogenic arthritis, pyoderma gangrenosum and acne (PAPA), adult-onset Still's disease (AOSD), Majeed syndrome, PLCG2-associated antibody deficiency and immune dysregulation (PLAID), PLCG2-associated autoinflammation, antibody deficiency and immune dysregulation (APLAID), pyogenic arthritis, haploinsufficiency of A20 (HA20), pediatric granulomatous arthritis (PGA), or sideroblastic anemia with B-cell immunodeficiency, periodic fevers, developmental delay (SIFD);
D. Skin disease, disorder, or abnormality such as hidradenitis suppurativa (HS), dermatitis, psoriasis, skin contact hypersensitivity, acne, periodic fever syndrome (HIDS), Sweet's syndrome, eczema, skin lesions, burn, wound, wound healing, trauma, sunburn, actinic keratosis, deficiency of interleukin 1 receptor (DIRA) antagonist, or alopecia areata;
E. Ocular disease, disorder, or abnormality, such as age-related macular degeneration (AMD), corneal infection, uveitis, glaucoma, dry eye, or demyelination;
F. Cardiovascular disease, disorder, or abnormality (e.g. disease, disorder, or abnormality of the cardiovascular system) such as myocardial infarction, hypertension, ischaemia reperfusion injury, pericarditis including Dressler's syndrome, aneurysms including abdominal aortic aneurism, or stroke;
G. Metabolic disease, disorder, or abnormality, such as type 2 diabetes, obesity, atherosclerosis, gout, or pseudo-gout;
H. Respiratory disease, disorder, or abnormality (e.g. disease, disorder or abnormality of the respiratory system), such as asbestosis, silicosis, cystic fibrosis, allergic inflammation, chronic obstructive pulmonary disorder (COPD), steroid-resistant asthma, or asthma;
I. Liver disease, disorder, or abnormality, (e.g. hepatic disease, disorder or abnormality) such as alcoholic liver disease, alcoholic fatty liver disease (AFLD), alcoholic steatohepatitis (ASH), non-alcoholic fatty liver disease (NAFLD), or non-alcoholic steatohepatitis (NASH) including advanced fibrosis stages F3 and F4;
J. Renal disease, disorder, or abnormality (e.g. disease, disorder or abnormality of the renal system) such as oxalate-induced nephropathy, diabetic nephropathy, chronic kidney disease, or kidney disease;
K. Cancer disease, disorder, or abnormality (e.g. cancer, tumor, or malignancy), such as lung cancer (e.g. lung cancer metastasis), pancreatic cancers, gastric cancers, leukemia, myelodysplastic syndrome (MOS), skin cancer, tumors of the endocrine system, or thyroid cancer;
L. Infections including viral infections, such as helminth infections (e.g. from schistosoma, roundworms, tapeworms or flukes), viral encephalitis, bacterial infection, human immunodeficiency virus (HIV), HIV-associated neurocognitive disorder, chronic nonbacterial osteomyelitis (CNO), chronic bacterial osteomyelitis, deficiency of interleukin 1 receptor (DIRA) antagonist, or epilepsy; alphavirus (e.g. Chikungunya virus and Ross River virus), flaviviruses (e.g. Dengue and Zika virus), Coronavirus-associated inflammatory pathologies, Coronaviruses, or influenza virus;
M. Psychological disease, disorder, or abnormality, such as depression, and psychological stress;
N. Inflammation, including inflammation occurring as a result of an inflammatory disease, disorder, or abnormality, such as an autoinflammatory disease, inflammation occurring as a symptom of a non-inflammatory disorder, inflammation occurring as a result of infection, or inflammation secondary to trauma, injury or autoimmunity. Examples of inflammation include inflammatory responses occurring in connection with, or as a result of:
   i. A joint disease, disorder, or abnormality, such as periodic fever syndrome (HIDS), rheumatoid arthritis, pustulosis, synovitis, osteoarthritis, chronic recurrent multifocal osteomyelitis (CRMO), systemic juvenile idiopathic arthritis, osteitis syndrome (SAPHO), hyperostosis, relapsing polychondritis, or adult-onset Still's disease;
   ii. A gastrointestinal disease, disorder, or abnormality (e.g. disease, disorder or abnormality of the gastrointestinal tract) such as colitis, ulcerative colitis, or inflammatory bowel disease;
   iii. A muscular disease, disorder, or abnormality, such as polymyositis, or myasthenia gravis;
   iv. A disease, disorder or abnormality of the endocrine system, such as, diabetes, parathyroid disease (e.g. hypothyroidism), tumors of the endocrine system, thyroid cancer, or hypoglycemia; and/or
   v. A vascular disease, disorder or abnormality, such as Behcet's disease.

In one embodiment, the disease, disorder, or abnormality is selected from Alzheimer's disease, Parkinson's disease, cryopyrin-associated periodic syndromes (CAPS), non-alcoholic fatty liver disease (NAFLD), non-alcoholic steatohepatitis (NASH), and gout.

In particular, the disease, disorder or abnormality is selected from: Alzheimer's disease, Parkinson's disease, amyotrophic lateral sclerosis, multiple sclerosis, demyelination, viral encephalitis, epilepsy, stroke, brain haemorrhage, atherosclerosis, asthma, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease, nonalcoholic fatty liver disease, nonalcoholic steatohepatitis, hypertension, myocardial infarction, oxalate-induced nephropathy, graft-versus host disease, type 1 diabetes, type 2 diabetes, rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, acne, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, acne, eczema, alopecia areata, actinic keratosis, hyperostosis, osteitis syndrome (SAPHO), multiple sclerosis (MS), psoriasis, Behcet's disease, Sjogren's syndrome, Schnitzler syndrome, chronic obstructive pulmonary disorder (COPD), steroid-resistant asthma, asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, chronic kidney disease, diabetic nephropathy, alcoholic liver disease, skin contact hypersensitivity, sunburn, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, hidradenitis suppurativa (HS), lung cancer metastasis, pancreatic cancers, gastric cancers, myelodisplastic syndrome, leukemia; polymyositis, colitis, helminth infection, bacterial infection, abdominal aortic aneurism, wound healing, depression, psychological stress, pericarditis including Dressler's syndrome, ischaemia reperfusion injury, frontotemporal dementia, HIV-associated neurocognitive disorder, Coronavirus-associated inflammatory pathologies, and traumatic brain and spinal cord injury, inflammatory pain, chronic pain, neuropathic pain, metastatic cancer-induced bone pain, chemotherapy induced peripheral neuropathy and migraine; preferably the disorder is selected from Alzheimer's disease, Parkinson's disease, amyotrophic lateral sclerosis, multiple sclerosis, demyelination, viral encephalitis, epilepsy, stroke, atherosclerosis, asthma, allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, Hereditary Recurrent Fevers (HRFs), and amyloidosis (including AL amyloidosis, AA amyloidosis, ATTR amyloidosis, hereditary amyloidoses (including apolipoprotein A-I (AApoAI), apolipoprotein A-II (AApoAll), gelsolin (AGeI), fibrinogen (AFib), and lysozyme (ALys)), Beta-2 Microglobulin amyloidosis, iAPP amyloidosis).

In one embodiment, the disease, disorder or abnormality is selected from: Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOMID), gout, pseudo-gout, inflammatory bowel disease, nonalcoholic fatty liver disease, nonalcoholic steatohepatitis, hypertension, myocardial infarction, oxalate-induced nephropathy, graft-versus host disease, type 1 diabetes, type 2 diabetes, rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, acne, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, acne, eczema, alopecia areata, actinic keratosis, hyperostosis, osteitis syndrome (SAPHO), multiple sclerosis (MS), psoriasis, Behcet's disease, Sjogren's syndrome, Schnitzler syndrome, chronic obstructive pulmonary disorder (COPD), steroid-resistant asthma, asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, chronic kidney disease, diabetic nephropathy, alcoholic liver disease, skin contact hypersensitivity, sunburn, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, hidradenitis suppurativa (HS), lung cancer metastasis, pancreatic cancers, gastric cancers, myelodisplastic syndrome, leukemia; polymyositis, colitis, helminth infection, bacterial infection, abdominal aortic aneurism, wound healing, depression, psychological stress, pericarditis including Dressler's syndrome, ischaemia reperfusion injury, frontotemporal dementia, HIV-associated neurocognitive disorder, Coronavirus-associated inflammatory pathologies, and traumatic brain injury; preferably the disorder is selected from Alzheimer's disease, Parkinson's disease, amyotrophic lateral sclerosis, demyelination, viral encephalitis, epilepsy, stroke, atherosclerosis, asthma, allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, and Hereditary Recurrent Fevers (HRFs).

In yet another embodiment, the disease, disorder or abnormality is preferably an inflammatory disease, disorder or abnormality; or an autoimmune disease, disorder or abnormality; or a disease, disorder or abnormality of the skin (such as, for example, but not limited to, psoriasis, acne, eczema, alopecia areata, or actinic keratosis); or a disease, disorder or abnormality of the cardiovascular system; or a disease, disorder, or abnormality such as a cancer, a tumor or a malignancy; or a disease, disorder or abnormality of the renal system; a disease, disorder or abnormality of the gastrointestinal tract; a disease, disorder or abnormality of the respiratory system; or a disease, disorder or abnormality of the endocrine system; or a disease, disorder or abnormality of the central nervous system (CNS); or a disease, disorder or abnormality of the liver.

### Definitions

Within the meaning of the present application the following definitions apply unless specified otherwise, and when appropriate, terms used in the singular will also include the plural and vice versa:

“Alkyl” refers to a saturated straight or branched organic moiety consisting of carbon and hydrogen atoms. Examples of suitable alkyl groups have 1 to 4 carbon atoms, preferably 1 to 3 carbon atoms, and (as appropriate) include methyl, ethyl, propyl, isopropyl, n-butyl, t-butyl, and isobutyl. The term “C₁-C₆alkyl” refers to an alkyl group having 1 to 6 carbon atoms. The terms“C₁-C₄alkyl”, “C₁-C₃alkyl”, or “C₁alkyl” are to be construed accordingly. “Hal”, “halo” or “halogen” refers to F, Cl, Br, and I. Preferably halogen is F or Cl. More preferably, halogen is Cl. Even more preferably, halogen is F.

“—O-C₁-C₆ alkyl” where “C₁-C₆alkyl” is as generally defined above. Examples of “—O-C₁-C₆ alkyl” include, but are not limited to methoxy, ethoxy, propoxy, isopropoxy, pentoxy, and hexoxy.

The term “C₃-C₆ cycloalkyl” refers to saturated monocyclic hydrocarbyl groups having 3 to 6 carbon atoms. The terms “C₅-C₆ cycloalkyl” is to be construed accordingly. Examples include, but are not limited to cyclopropyl, cyclobutyl, cyclopentyl, and cyclohexyl.

“4-, 5- or 6-membered heterocycloalkyl” refers to a stable 4-, 5- or 6-membered non-aromatic monocyclic ring radical which comprises 1 or 2 heteroatoms. The heteroatom is nitrogen. Examples include piperidine.

“Aryl” refers to an aromatic hydrocarbon group having, 5 or 6 carbon atoms in the ring portion. Preferably, “aryl” is phenyl.

“Heteroaryl” refers to an aromatic “5 or 6-membered ring”, wherein one or two of the carbon atoms in the ring have been replaced by heteroatoms. In the context of the present invention the heteroatoms is N. The heteroaryl radical may be bonded via a carbon atom or heteroatom. Examples of heteroaryl include, but are not limited to, pyridine and pyrimidine.

“Optionally substituted” in reference to a certain group refers to said group as to optionally be substituted with one or more substituents (i.e. the substituent may be present or not).

Unless specified otherwise, the term “compound of the present invention” refers to compounds of formula (I′), (I), (Ia), (Ib), (Ic), (Id) or (Ie) as disclosed herein, or sub-formulae thereof, as disclosed herein, or stereoisomers thereof, or racemic mixtures thereof, or tautomers thereof, or polymorphs thereof, or pharmaceutically acceptable salts thereof, or prodrugs thereof, or hydrates thereof, or solvates thereof. Compounds of the present invention having one or more optically active carbons can exist as racemates and racemic mixtures (including mixtures in all ratios), stereoisomers (including diastereomeric mixtures and individual diastereomers, enantiomeric mixtures and single enantiomers, mixtures of conformers and single conformers), tautomers, atropisomers, and rotamers. All isomeric forms are included in the present invention. Compounds described in this invention containing olefinic double bonds include E and Z geometric isomers. Also included in this invention are all pharmaceutically acceptable salts, prodrugs, hydrates and solvates of compounds of formula (I′), (I), (Ia), (Ib), (Ic), (Id) or (Ie).

Tautomers are isomers of a compound which differ only in the position of the protons and electrons. The skeleton of the compound is unchanged. Common tautomeric pairs include: ketone—enol (H—O—C═CH⇄O═C—CH₂), enamine—imine (H₂N—C═N⇄HN═C—NH).

Solvates, hydrates as well as anhydrous forms of the salt are also encompassed by the invention. The solvent included in the solvates is not particularly limited and can be any pharmaceutically acceptable solvent. Examples include water and C₁₋₄ alcohols (such as methanol or ethanol).

“Pharmaceutically acceptable salts” are defined as derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as, but not limited to, hydrochloric, hydrobromic, sulfuric, sulfamic, phosphoric, nitric acid and the like; and the salts prepared from organic acids such as, but not limited to, acetic, propionic, succinic, glycolic, stearic, lactic, malic, tartaric, citric, ascorbic, palmoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic acid, and the like. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Organic solvents include, but are not limited to, nonaqueous media like ethers, ethyl acetate, ethanol, isopropanol, or acetonitrile. Lists of suitable salts can be found in Remington's Pharmaceutical Sciences, 18^{th} ed., Mack Publishing Company, Easton, P A, 1990, p. 1445, the disclosure of which is hereby incorporated by reference.

The compounds of the present invention, as defined herein, can also be provided in the form of a prodrug, namely a compound which is metabolized in vivo to the active metabolite. As used hereinafter in the description of the invention and in the claims, the term “prodrug” means any covalently bonded compound which releases the active parent pharmaceutical due to in vivo biotransformation. The reference by Goodman and Gilman (The Pharmacological Basis of Therapeutics, 8 ed, McGraw-Hill, Int. Ed. 1992, “Biotransformation of Drugs”, p 13-15) describing prodrugs generally is hereby incorporated herein by reference.

“Pharmaceutically acceptable” is defined as those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

A used herein, the terms “patient” or “subject” mentioned in the present invention typically refer to an animal, particularly a mammal (e.g. rabbits, rats, dogs, mice, guinea pigs, pigs), more particularly primates (e.g. humans, male or female). In certain embodiments, the subject is a human.

“NLRP3” as used herein refers to NOD-like receptor (NLR) family, pyrin-domain containing protein 3 component of inflammasome. Inflammasomes are intracellular supramolecular complexes comprising a sensor molecule, the adaptor apoptosis-associated speck-like protein containing a CARD (ASC) and the effector protease caspase 1. Upon activation of the inflammasome sensor molecule, ASC self-associates into a helical fibrillary assembly resulting in formation of the so-called ASC speck or pyroptosome, which acts as a molecular platform for the activation of pro-caspase 1 via proximity-induced autocatalytic activation. Active caspase 1 triggers the activation and release of interleukin-1 (IL-1) family proteins and enables the non-conventional secretion of numerous cytosolic proteins. Among the pro-inflammatory mediators released upon NLRP3 activation are IL-1 beta (β), IL-18, high-mobility group protein B1 (HMGB1), leukotrienes and prostaglandins.

NLRP3 inflammasome pathway activation is an important driver of inflammation interacting with the different cytokine pathways shaping the immune response to infection and injury. Formation of some pro-inflammatory cytokines is triggered by NLRP3 inflammasome pathway activation.

The terms “inhibit”, “inhibition” or “inhibiting” refer to the reduction or suppression of a given condition, symptom, or disorder, or disease, or abnormality which is responsive to the modulation of a component of the NLRP3 inflammasome pathway, or a significant decrease in the baseline activity of a biological activity or process.

The terms “treat”, “treating” or “treatment” of any disease, disorder or abnormality refer to alleviating or ameliorating or modulating the disease or disorder or abnormality (i.e., slowing or arresting the development of the disease, disorder or abnormality or at least one of the clinical symptoms thereof); or alleviating or ameliorating or modulating at least one physical parameter or biomarker associated with the disease or disorder or abnormality, including those which may not be discernible to the subject (e.g., patient).

The terms “prevent”, “preventing” or “prevention” of any disease or disorder or abnormality which is responsive to the modulation of a component of the NLRP3 inflammasome pathway refer to the prophylactic treatment of the disease or disorder or abnormality; or delaying the onset or progression of the disease or disorder.

The term “in need of” a treatment if such subject would benefit biologically, medically or in quality of life from such treatment.

As used herein, “modulation” refers to alteration, e.g., up-regulation, down-regulation, increase or decrease, preferably decrease.

| Abbreviation | Meaning |
|---|---|
| NALP1-14 | NACHT Leucine-rich-repeat Protein 1-14 (a synonym |
| | of NLRP) |
| IPAF | Ice Protease-Activating Factor |
| NAIP | Neuronal Apoptosis Inhibitory Protein |
| ASC | Apoptosis-associated Speck-like protein containing |
| | a CARD |
| nucleotide- | NACHT: NAIP (neuronal apoptosis inhibitory |
| binding NACHT | protein), CIITA (MHC class II transcription |
| domain | activator), HET-E (incompatibility locus protein |
| | from Podospora anserina) and TP1 (telomerase- |
| | associated protein) |
| IL | Interleukin |
| TNF-alpha | Tumor Necrosis Factor-alpha |

The definitions and preferred definitions given in the “Definition”-section apply to all of the embodiments described herein unless stated otherwise.

General Synthetic Scheme for the Preparation of Compounds of this Invention

The compounds of the present invention can be synthesized by those skilled in the art by using commonly known preparation steps, for instance those of the general methods shown in the following schemes. These methods are only given for illustrative purposes and should not be construed as limiting.

In all of the methods it is understood that protecting groups for sensitive or reactive groups may be employed where necessary in accordance with general principles of chemistry. Protecting groups are manipulated according to standard methods of organic synthesis (T. W. Green and P. G. M. Wuts (2014) Protective Groups in Organic Synthesis, 5th edition, John Wiley & Sons). These groups are removed at a convenient stage of the compound synthesis using methods that are readily apparent to those skilled in the art.

In the following general methods, R₀, R₁, R₂, R₃, Z₁, Z₂, E, V, X and Y are as previously defined in the above embodiments.

| Abbreviation | Meaning |
|---|---|
| DCM | Dichloromethane |
| DIPEA | N,N-Diisopropylethylamine |
| DMSO | Dimethylsulfoxide |
| EtOAc | Ethyl acetate |
| EtOH | Ethanol |
| HPLC | High performance liquid chromatography |
| NMR | Nuclear magnetic resonance |
| rt or RT | Room temperature |
| SM | Starting material |
| THF | Tetrahydrofuran |
| UPLC | Ultra Performance Liquid Chromatography |
| CuMeSal | Copper(I) 3-methylsalicylate |
| PE | Petroleum ether |
| br | Broad |
| Lawesson's | 2,4-Bis(4-methoxyphenyl)-1,3,2,4-dithiadiphosphetane- |
| reagent | 2,4-disulfide |
| LiHMDS | Lithium bis(trimethylsilyl)amide |
| Py | Pyridine |
| DMF | N,N-Dimethylformamide |
| ACN | Acetonitrile |
| SNAr | Aromatic Nucleophilic Substitution |
| Hal | Halogen |
| Lvg | Leaving group |

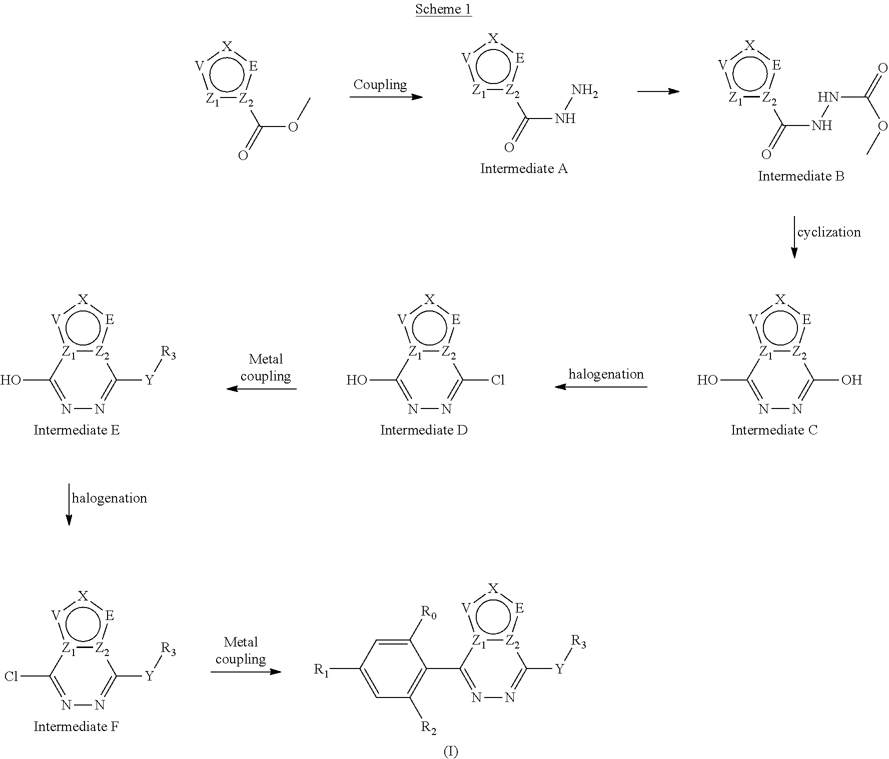

Commercially available 5-membered heterocyclic ester can be condensed with hydrazine followed by carbamate formation allowing the synthesis of the intermediate B. Ring cyclization can be conducted under basic conditions such as KOH in an appropriate solvent to afford bicyclic system intermediate C. Subsequent halogenation using POCl₃ or any halogenating reagent can deliver intermediate D. Introduction of YR₃ substituent can be performed by palladium catalysed amination using standard palladium catalyst, base and solvent. Intermediate E can be submitted to halogenation using POCl₃ or any halogenating reagent. Finally, intermediate F can be further functionalized using palladium catalysed Suzuki reaction to give compounds of formula (I′), (I), (Ia), (Pb), (Ic), (Id) or (Ie).

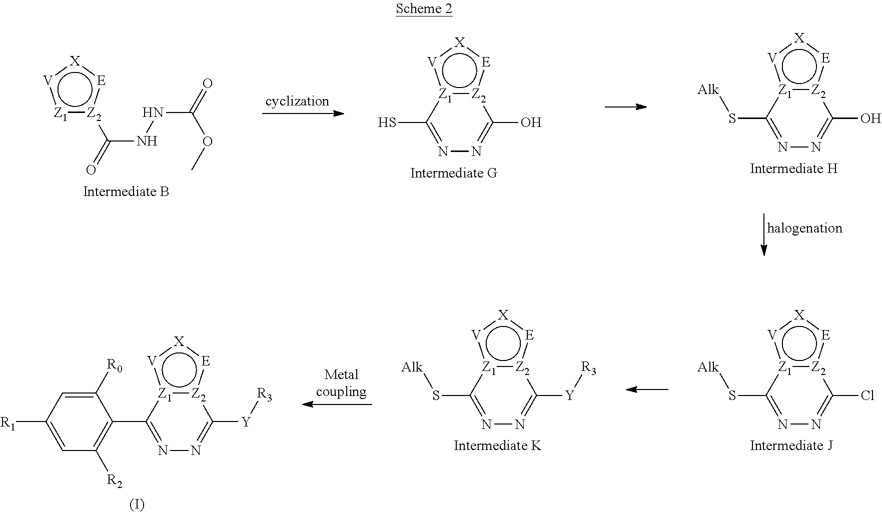

Starting from Intermediate B, ring cyclization using CS₂ in presence of base can deliver intermediate G. Selective alkylation using standard conditions followed by chlorination can give intermediate J. Finally, SNAr followed by metal coupling using, for example, boronic acid and palladium catalyst can offer a compound of formula (I).

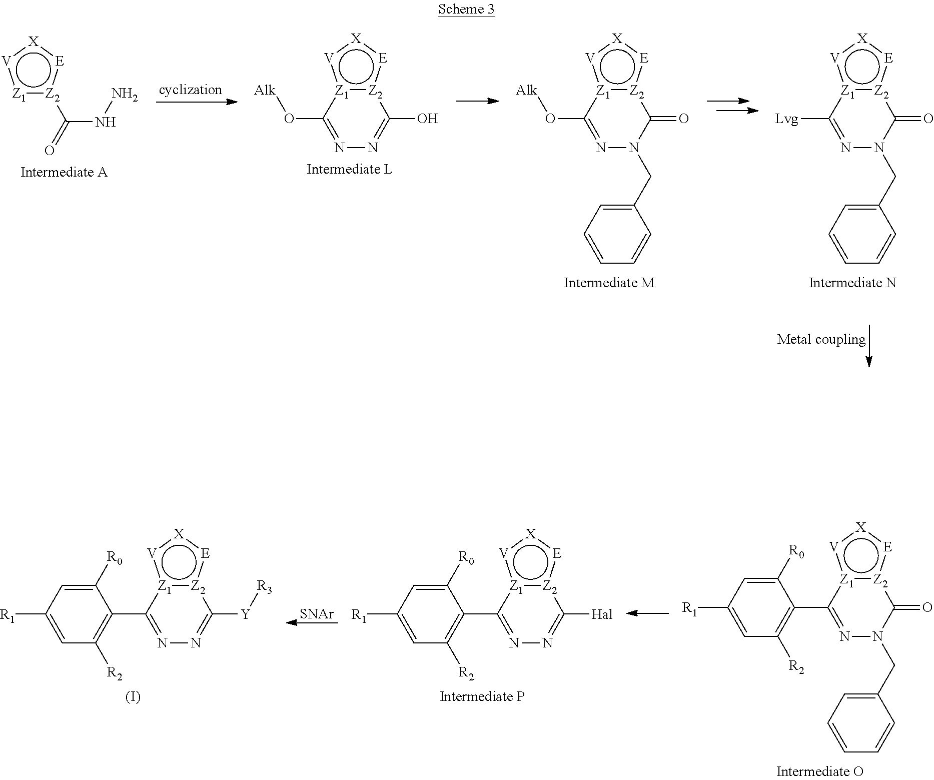

Another approach can include cyclization of Intermediate A with orthoformate regeant to give Intermediate L. Benzylation of the scaffold using standard conditions can deliver Intermediate M. Then, alkylether can be converted in a leaving group by a two-step approach. Intermediate O can be obtained by metal coupling starting from intermediate N. Finally, pyridone is converted to a leaving group in a one pot reaction to give Intermediate P. Finally, compounds of Formula (I) can be obtained by SNAr reaction using standrad conditions.

### EXAMPLES

The disclosure is further illustrated by the following examples and synthesis schemes, which are not to be construed as limiting the scope of the specific procedures herein described. It is understood that the examples are provided to illustrate certain embodiments and that no limitation to the scope of this disclosure is intended thereby.

Unless otherwise noted, all reagents and solvents were obtained from commercial sources and used without further purification. The chemical names were generated using ChemDraw from CambridgeSoft. Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. ¹H-NMR spectra were recorded on Bruker 400 MHz-Avance Neo Nanobay NMR spectrometers in deuterated solvents. Chemical shifts (δ) are reported in parts per million and coupling constants (J values) in hertz. Spin multiplicities are indicated by the following symbols: s (singlet), d (doublet), t (triplet), q (quartet), m (multiplet), bs (broad singlet). Mass spectra (MS) were obtained on a Waters Alliance HPLC (Waters e2695 separation module), Waters Arc HPLC or Waters Acquity UPLC. Chromatography was performed using silica gel (SRL: Silica gel 100-200 mesh) and suitable solvents as indicated in specific examples. Flash purification was conducted with a CombiFlash system and the solvent gradient indicated in specific examples. Thin layer chromatography (TLC) was carried out on silica gel plates with UV detection.

Intermediate 1

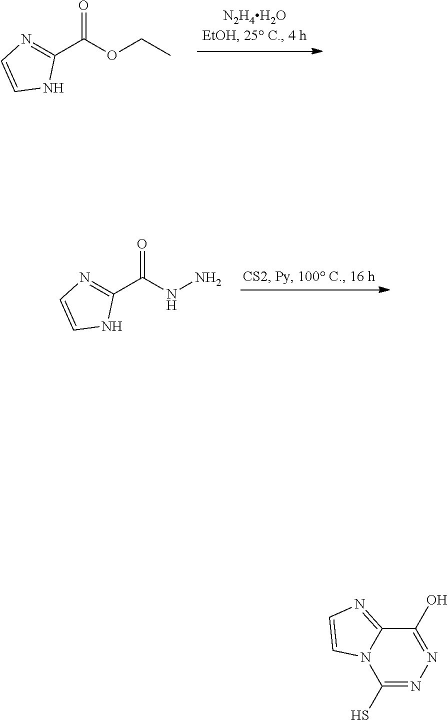

Step 1: To a mixture of ethyl 1H-imidazole-2-carboxylate (100 g, 713.57 mmol, 1 eq) in EtOH (1 L) was slowly added N₂H₄·H₂O (225.41 mL, 3.72 mol, 5.21 eq). The mixture was stirred at 25° C. for 4 h. The reaction mixture was filtered. The filter cake was washed with H₂O (300 mL×3) and dried to afford 1H-imidazole-2-carbohydrazide (70 g, 77%) as a white solid.

¹H NMR: (400 MHz, DMSO-d6) δ=12.64 (br s, 1H), 9.66 (s, 1H), 7.14 (s, 2H), 4.60 (s, 2H).

MS: 127.3 [M+H]⁺.

Step 2: To a mixture of 1H-imidazole-2-carbohydrazide (60 g, 475.75 mmol, 1 eq) in pyridine (1.6 L) was added CS₂ (573.60 mL, 8.52 mol, 20.0 eq). The mixture was stirred at 100° C. for 16 hours. The reaction mixture was concentrated under vacuum, the residue was diluted with H₂O (500 mL), then the pH was adjusted to pH=2 with 1M HCl. The resultant yellow solid was filtered and dried. The crude product was triturated with MeOH (200 mL) at 25° C. for 16 h. The mixture was filtered and the filter cake was washed with MeOH (20 mL*3) and lyophilized to afford 5-mercaptoimidazo[1,2-d][1,2,4]triazin-8-ol (25.5 g, 31%) as a yellow solid.

¹H NMR: (400 MHz, DMSO-d6) δ=8.21 (d, 1H), 7.72 (d, 1H).

MS: 169.0 [M+H]⁺.

### Example 1: (R)-2-(14(1-methylpiperidin-3-yl)amino)pyrrolo[1,2-cl][1,2,4]triazin-4-0)-5-(trifluoromethyl)phenol

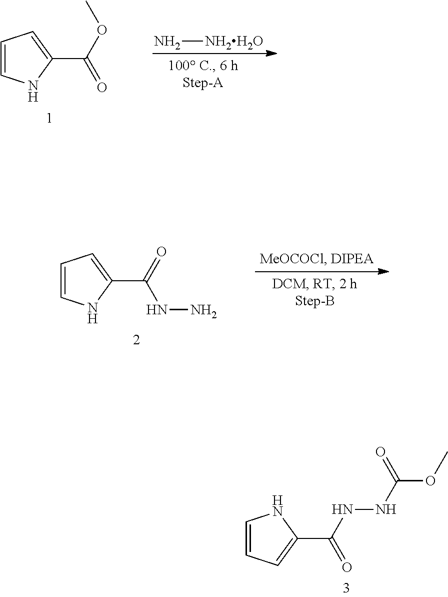

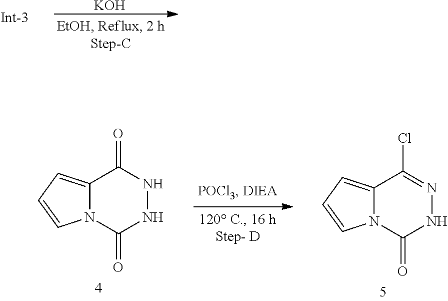 [SMILES: "KOH" ; O=c1[nH][nH]c(=O)n2cccc12 ; O=c1[nH]nc(Cl)c2cccn12]

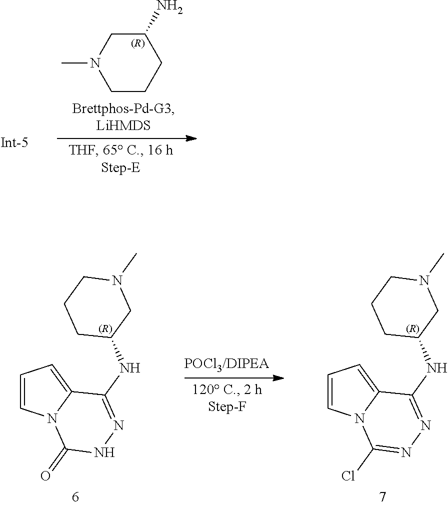

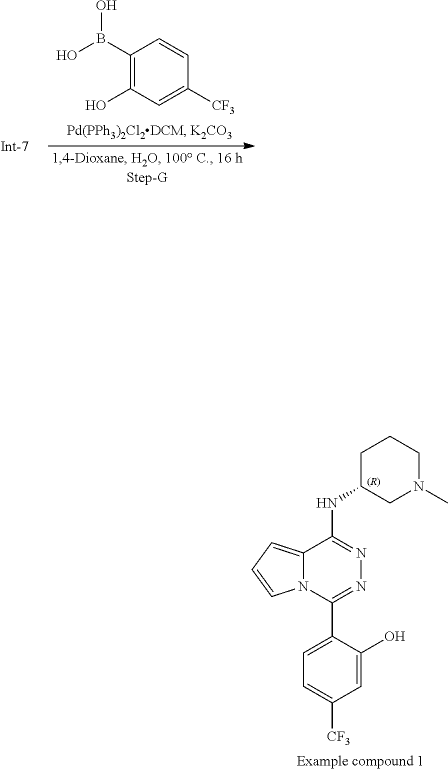

Step A: A solution of methyl 1H-pyrrole-2-carboxylate (10 g, 79.92 mmol) in hydrazine hydrate (76.86 mL, 2397.51 mmol) was stirred at RT for 1 h, then heated to 100° C. for 2 h. After the consumption of the starting material, the reaction mixture was diluted with H₂O (50 mL), solid was precipitated on cooling. The solid was filtered through a Büchner funnel, washed with H₂O (20 mL) and dried to afford 1H-pyrrole-2-carbohydrazide (8.0 g, 80%) as an off-white solid.

¹H NMR (400 MHz, CDCl₃): δ=11.42 (brs, 1H), 9.22 (brs, 1H), 6.83-6.82 (m, 1H), 6.73 (brs, 1H), 6.05-6.03 (m, 1H), 4.28-4.27 (brs, 2H).

MS: 125.98 [M+H]+.

Step B: To a stirred solution of 1H-pyrrole-2-carbohydrazide (obtained from step A) (8.0 g, 63.93 mmol) and N,N-diisopropylethylamine (33.41 ml, 191.80 mmol) in DCM (350 mL) was added methyl chloroformate (7.428 ml, 95.90 mmol) at room temperature dropwise over 15 min. The mixture was stirred at room temperature for 2 h. After the consumption of the starting material, The RM was evaporated under reduced pressure to get the crude compound. The crude compound was co-distilled with DCM (50 mL) and dried thoroughly under vacuum to get the crude product methyl 2-(1H-pyrrole-2-carbonyl)hydrazine-1-carboxylate (8.0 g, 68%) as an oil.

MS: 184.07 [M+H]+.

Step C: To a stirred solution of methyl 2-(1H-pyrrole-2-carbonyl)hydrazine-1-carboxylate (obtained from step B) (8.0 g, 43.68 mmol) in EtOH (800 mL) was added KOH (8.58 g, 152.87 mmol) and heated to reflux for 2 h. The reaction was monitored by TLC. After consumption of the starting material, the reaction mixture was cooled to room temperature. The precipitate formed was filtered and the solid was taken in H₂O (50 mL), then acidified with 1.0 N HCl (aq.) to pH-4 and extracted with EtOAc (2×300 mL). The combined organic layers were evaporated under reduced pressure to afford 2,3-dihydropyrrolo[1,2-d][1,2,4]triazine-1,4-dione (6.3 g, 95%) as an off-white solid.

¹H NMR (400 MHz, DMSO-d6): δ=11.42 (s, 2H), 7.67-7.66 (s, 1H), 6.81-6.80 (m, 1H), 6.73-6.71 (m, 1H).

MS: 152.04 [M+H]⁺.

Step D: To a stirred solution of 2,3-dihydropyrrolo[1,2-d][1,2,4]triazine-1,4-dione (obtained from step C) (2.5 g, 16.54 mmol) in POCl₃ (15.56 mL, 165.42 mmol) was added DIPEA (2.3 mL, 13.23 mmol) dropwise at room temperature and the mixture was stirred at 120° C. for 16 h. The progress of the reaction was monitored by TLC. After the consumption of the starting material, the reaction mixture was added to ice in a dropwise manner under stirring and then stirred for 15 min. The off-white solid was filtered off through a Büchner funnel and dried to afford the desired product 1-chloropyrrolo[1,2-d][1,2,4]triazin-4(3H)-one (1.8 g, 64%) as an off-white solid.

¹H NMR (400 MHz DMSO-d6): δ=12.53 (s, 1H), 7.88-7.86 (m, 1H), 6.92-6.87 (m, 2H).

MS: 168.01 [M−H]⁻.

Step E: To a stirred degassed solution of 1-chloropyrrolo[1,2-d][1,2,4]triazin-4(3H)-one (obtained from step D) (1.0 g, 5.93 mmol) and (R)-1-methylpiperidin-3-amine (1.36 g, 11.86 mmol) in tetrahydrofuran (10 mL) were added BrettPhos Pd G3 (0.54 g, 0.59 mmol) and BrettPhos (0.318 g, mmol) followed by lithium bis(trimethylsilyl)amide, 1M solution in THF, (17.80 ml, 17.80 mmol) in one charge at RT. The mixture was stirred at 65° C. for 16 h. The reaction was monitored by TLC. After the consumption of the starting material, the reaction mixture was quenched with saturated NH₄Cl solution (70 mL) and extracted with ethyl acetate (2×200 mL). The combined organic layers were dried over Na₂SO₄, filtered and evaporated under reduced pressure to get the crude compound as a pale brown solid. The crude compound was triturated with diethylether (2×20 mL) to get a brown solid. The resulting solid was filtered through a Büchner funnel and dried to afford (R)-14(1-methylpiperidin-3-yl)amino)pyrrolo[1,2-d][1,2,4]triazin-4(3H)-one (1.2 g, 82%) as a pale brown solid.

¹H NMR (400 MHz, DMSO-d6): δ=11.20 (s, 1H), 7.60-7.59 (q, 1H), 6.99-6.98 (dd, 1H), 6.67-6.65 (t, 1H), 6.28-6.26 (d, 1H), 5.75 (s, 1H), 3.78-3.71 (m, 1H), 2.96-2.94 (m, 1H), 2.65-2.62 (m, 1H), 2.15 (s, 3H), 1.91-1.66 (m, 4H), 1.55-1.45 (m, 1H), 1.30-1.20 (m, 1H).

MS: 248.11 [M+H]⁺.

Step F: To a stirred solution of (R)-1-((1-methylpiperidin-3-yl)amino)pyrrolo[1,2-d][1,2,4]triazin-4(3H)-one (obtained from step E) (500 mg, 2.02 mmol) in POCl₃ (3 mL) was added N,N-diisopropylethylamine (0.28 mL, 1.62 mmol) dropwise at room temperature and the mixture was stirred at 120° C. for 6 h. The reaction was monitored by TLC. After the consumption of the starting material, the reaction mixture was evaporated, quenched with ice, basified with saturated sodium bicarbonate solution (50 ml) and extracted with EtOAc (2×150 mL). The combined organic layers were washed with brine solution (100 mL), dried over anhydrous sodium sulfate, filtered and concentrated under reduced pressure to get the crude product (R)-4-chloro-N-(1-methylpiperidin-3-yl)pyrrolo[1,2-d][1,2,4]triazin-1-amine (380 mg, 70%) as an off-white gum.

MS: 266.06 [M+H]⁺.

Step G: To a stirred degassed solution of (R)-4-chloro-N-(1-methylpiperidin-3-yl)pyrrolo[1,2-d][1,2,4]triazin-1-amine (obtained from step F) (250 mg, 0.94 mmol) and (2-hydroxy-4-(trifluoromethyl)phenyl)boronic acid (232.47 mg, 1.13 mmol) in 1,4-dioxane (3 mL) and water (3 mL) were added potassium carbonate (390.18 mg, 2.82 mmol) and 1,1′-bis(diphenylphosphino)ferrocene-palladium(11)dichloride dichloromethane adduct (76.78 mg, 0.27 mmol) at room temperature and the mixture was heated to 100° C. for 16 h. The reaction was monitored by TLC. After the consumption of the starting material, the reaction mixture was diluted with water (20 mL) and extracted with EtOAc (2×30 mL). The combined organic layers were separated, washed with brine solution (20 mL), dried over anhydrous sodium sulfate, filtered and concentrated under reduced pressure to get the crude compound. The crude compound was purified by preparative HPLC purification.

Preparative HPLC Purification Conditions

Mobile phase: 10 mM ammonium bicarbonate in water/acetonitrile

Column: mobile phase—lnertsil ODS-3V (25×150) mm 10 μm

Flow: 18 mL/min

Gradient method: T/%B-0/30, 4155, 12/55, 12.05/99, 14.05/99, 14.10/30.18.10/30.

Pure fractions were collected and lyophilized to afford (R)-2-(1-((1-methylpiperidin-3-yl)amino)pyrrolo[1,2-d][1,2,4]triazin-4-yl)-5-(trifluoromethyl)phenol (22 mg, 6%) as an off-white solid.

¹H NMR (400 MHz, DMSO-d6): δ=10.81 (brs, 1H), 7.65-7.63 (d, 1H), 7.32-7.29 (m, 2H), 7.08-7.04 (dd, 2H), 6.99-6.97 (d, 1H), 6.72-6.70 (t, 1H), 4.25 (brs, 1H), 3.04-3.02 (d, 1H), 2.71-2.67 (brs, 1H), 2.21 (s, 3H), 1.92-1.90 (m, 3H), 1.76-1.72 (m, 1H), 1.62-1.56 (m, 1H), 1.41-1.36 (m, 1H),

MS: 392.26 [M+H]⁺.

### Examples 2 to 5

Analogously to the above procedure of example 1, the below examples were synthesized:

| | | | Mass |
|---|---|---|---|
| Example | IUPAC name | NMR | [M + H]⁺ |
| 2 | (R)-2-(4-((1-methylpiperidin-3- | ¹H NMR (400 MHz, | 393.26 |
| 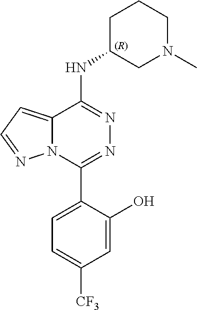 | yl)amino)pyrazolo[1,5- d][1,2,4]triazin-7-yl)-5- (trifluoromethyl)phenol | DMSO) δ 8.28 (d, 1H), 8.17 (s, 1H), 7.63 (d, 1H), 7.32-7.22 (m, 3H), 4.27- 4.22 (m, 1H), 3.03 (d, 1H), 2.70-2.63 (m, 1H), 2.32 (s, 3H), 2.05-1.85 (m, 3H), 1.78-1.66 (m, 1H), 1.59 (q, 1H), 1.44-1.35 (m, 1H). | |
| 3 | (R)-4-(4-(difluoromethoxy)phenyl)- | ¹H NMR (400 MHz, | 374.28 |
| 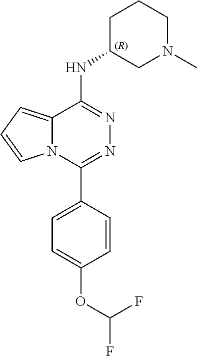 | N-(1-methylpiperidin-3- yl)pyrrolo[1,2-d][1,2,4]triazin-1- amine | DMSO) δ 7.88-7.79 (m, 2H), 7.43 (dd, 1H), 7.40- 7.32 (m, 2H), 7.13 (dd, 1H), 6.97 (d, 1H), 6.79 (dd, 1H), 4.25 (d, 1H), 3.01 (d, 1H), 2.68 (d, 1H), 2.19 (s, 3H), 1.99-1.78 (m, 3H), 1.78-1.66 (m, 1H), 1.57 (q, 1H), 1.37 (td, 1H). | |
| 4 | (R)-N-(1-methylpiperidin-3-yl)-4-(4- | ¹H NMR (400 MHz, | 392.25 |
| 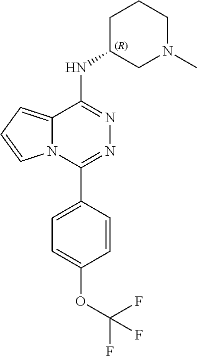 | (trifluoromethoxy)phenyl)pyrrolo [1,2-d][1,2,4]triazin-1-amine | DMSO) δ 7.92 (d, 2H), 7.62-7.52 (m, 2H), 7.46 (dd, 1H), 7.14 (dd, 1H), 7.02 (d, 1H), 6.80 (dd, 1H), 4.36-4.15 (m, 1H), 3.01 (d, 1H), 2.74-2.62 (m, 1H), 2.19 (s, 3H), 2.03- 1.80 (m, 3H), 1.80-1.66 (m, 1H), 1.57 (q, 1H), 1.47-1.28 (m, 1H). | |
| 5 | (R)-2-(8-((1-methylpiperidin-3- | ¹H NMR (400 MHz, | 393.24 |
| 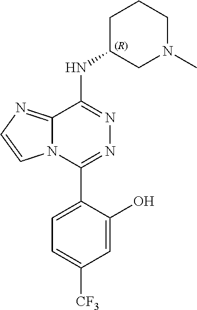 | yl)amino)imidazo[1,2- d][1,2,4]triazin-5-yl)-5- (trifluoromethyl)phenol | DMSO) δ 7.59-7.40 (m, 3H), 7.05 (d, 1H), 6.91 (s, 1H), 6.85-6.66 (m, 1H), 4.39-4.22 (m, 1H), 3.21- 3.08 (m, 1H), 2.82 (d, 1H), 2.27-1.95 (m, 5H), 1.87- 1.48 (m, 4H). | |

### Example 6: 5-(4-Chlorophenyl)-N-[(3R)-1-methyl-3-piperidyl]imidazo[1,2-d][1,2,4]triazin-8-amine

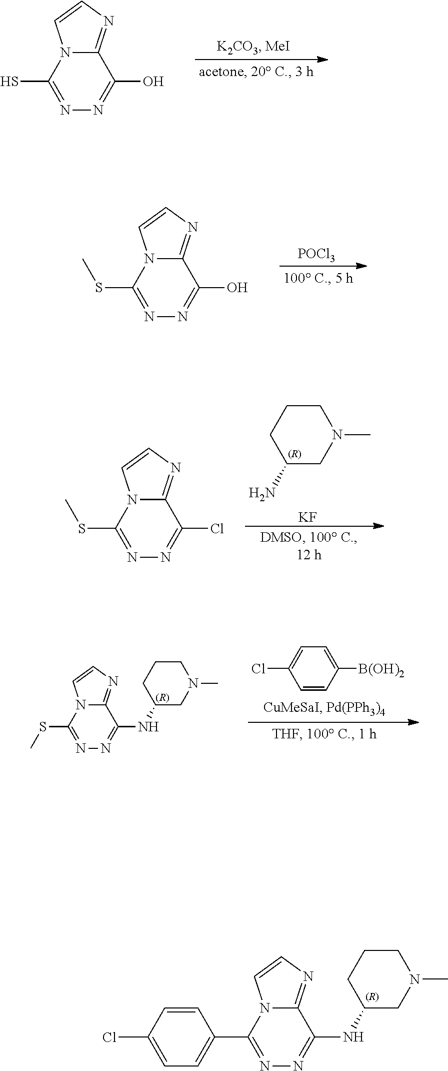

Step 1: To the solution of 5-sulfanylimidazo[1,2-d][1,2,4]triazin-8-ol (intermediate 1) (15 g, 89.19 mmol, 1.0 eq) in acetone (150 mL) was added K₂CO₃ (24.65 g, 178.38 mmol, 2 eq) and Mel (15.19 g, 107.03 mmol, 6.66 mL, 1.2 eq). The resulting reaction mixture was stirred at 20° C. for 3 h. The reaction solution was filtered to remove K₂CO₃ and then concentrated to give the crude product. The crude product was triturated with DCE (1000 mL) at 80° C. for 30 min. 5-Methylsulfanyl-imidazo[1,2-d][1,2,4]triazin-8-ol (12 g, crude) was obtained as a yellow solid.

¹H NMR (400 MHz, DMSO-d6) δ=12.62 (br s, 1H), 7.88 (d, 1H), 7.62 (d, 1H), 2.63 (s, 3H).

MS: 183.1 [M+H]⁺.

Step 2: A solution of 5-methylsulfanylimidazo[1,2-d][1,2,4]triazin-8-ol (4 g, 21.95 mmol, 1 eq) in POCl₃ (58.49 g, 381.46 mmol, 35.56 mL, 17.38 eq) was heated at 100° C. for 5 h. The reaction mixture was poured onto 100 mL saturated NaHCO₃ and followed by 20 mL DCM. After that, the aqueous phase was separated and extracted with DCM (30 mL×3). The combined organic layers were washed successively with water (20 mL×2) and brine (20 ml×1), dried over anhydrous Na₂SO₄, filtered, and concentrated to give a residue. The residue was purified by column chromatography (SiO₂, DCM: MeOH=100/1 to 10/1). 8-Chloro-5-methylsulfanyl-imidazo[1,2-d][1,2,4]triazine (4.4 g, 50% yield) was obtained as a yellow solid.

¹H NMR (400 MHz, DMSO-d6) δ=8.22 (d, 1H), 7.97 (d, 1H), 2.84 (s, 3H).

MS: 200.09 [m+H]⁺.

Step 3: To the solution of 8-chloro-5-methylsulfanyl-imidazo[1,2-d][1,2,4]triazine (2 g, 9.97 mmol, 1.0 eq.) in DMSO (10 mL) was added (3R)-1-methylpiperidin-3-amine (1.37 g, 11.96 mmol, 1.2 eq) and KF (1.16 g, 19.94 mmol, 467.01 μL, 2 eq.). The resulting reaction mixture was stirred at 100° C. for 12 h. The reaction mixture was poured onto 30 mL saturated Na₂CO₃. Then, the resulting solution was extracted with EtOAc (20 mL×6) (maintaining pH=12 of the aqueous phase). The combined organic layers were washed successively with saturated Na₂CO₃ (20 mL×3) and brine (20 ml×3) (maintaining pH=12 of the aqueous phase), dried over anhydrous Na₂SO₄, filtered, and concentrated to give a residue. The residue was purified by flash silica gel chromatography (SiO₂, DCM/MeOH=100/1 to 5/1) and then re-purified by prep-HPLC (column: Waters Xbridge BEH C18 250×70 mm×10 μm; mobile phase: [H₂O (0.05% NH₃H₂O)-ACN]; gradient: 15%-45% B over min) to give the title product. N-[(3R)-1-Methyl-3-piperidyl]-5-methylsulfanyl-imidazo[1,2-d][1,2,4]triazin-8-amine (2.34 g, 42% yield) was obtained as a yellow oil.

¹H NMR (400 MHz, DMSO-d6) δ=7.88 (d, 1H), 7.67 (d, 1H), 7.02 (br d, 1H), 4.24-4.13 (m, 1H), 2.84-2.76 (m, 1H), 2.71 (s, 3H), 2.55-2.53 (m, 1H), 2.21-2.16 (s, 3H), 2.11-1.99 (m, 2H), 1.80-1.74 (m, 1H), 1.67 (br dd, 1H), 1.58-1.47 (m, 2H).

MS: 279.2 [M+H]⁺.

Step 4: To the solution of N-[(3R)-1-methyl-3-piperidyl]-5-methylsulfanyl-imidazo[1,2-d][1,2,4]triazin-8-amine (0.3 g, 1.08 mmol, 1.0 eq) in THF (5 mL) was added (4-chlorophenyl)boronic acid (252.78 mg, 1.62 mmol, 1.5 eq), cuprous; 2-hydroxy-3-methyl-benzoate (578.40 mg, 2.69 mmol, 2.5 eq), Pd(PPh₃)₄ (373.60 mg, 323.30 μmol, 0.3 eq). The resulting reaction mixture was stirred at 100° C. for 1 h. The reaction mixture was poured onto 20 mL saturated EDTA and followed by 10 mL EtOAc.

The solution was stirred at 20° C. for 0.5 h. After that, the aqueous phase was separated and extracted with EtOAc (20 mL×5). The combined organic layer was washed successively with saturated Na₂CO₃ (20 mL×1), dried over anhydrous Na₂SO₄, filtered, and concentrated to give a residue. The residue was purified by column chromatography (SiO₂, Petroleum ether/Ethyl acetate=3/1 to DCM/MeOH=10/1) which resulted in 0.5 g of a 20% purity product. Re-purity by prep-HPLC (column: Phenomenex Luna C18 100×30 mm×5 μm; mobile phase: [H₂O(0.2% FA)-ACN]; gradient: 5%-30% B over 8.0 min) to give the pure product. 5-(4-Chlorophenyl)-N-[(3R)-1-methyl-3-piperidyl]imidazo[1,2-d][1,2,4]triazin-8-amine (45 mg, 12% yield) was obtained as an off-white solid.

¹H NMR (400 MHz, DMSO) δ 7.92 (s, 1H), 7.87 (d, 2H), 7.71-7.63 (m, 3H), 7.31 (d, 1H), 4.41-4.23 (m, 1H), 2.84 (d, 1H), 2.60-2.51 (m, 1H), 2.22 (s, 3H), 2.11 (d, 2H), 1.79 (s, 1H), 1.74-1.66 (m, 1H), 1.66-1.49 (m, 2H).

MS: 343.1 [M+H]⁺.

### Example 7: 5-Chloro-2-[8-[[(3R)-1-methyl-3-piperidyl]aminolimidazo[1,2-d][1,2,4]triazin-5-yl]phenol formate salt

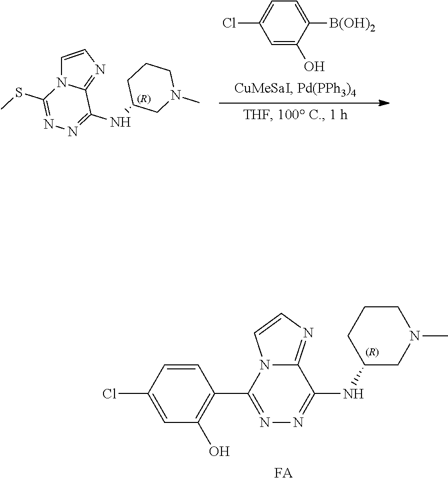

To the solution of N-[(3R)-1-methyl-3-piperidyl]-5-methylsulfanyl-imidazo[1,2-d][1,2,4]triazin-8-amine (0.3 g, 1.08 mmol, 1.0 eq) in THF (5 mL) was added (4-chloro-2-hydroxy-phenyl)boronic acid (278.65 mg, 1.62 mmol, 1.5 eq), cuprous; 2-hydroxy-3-methyl-benzoate (578.40 mg, 2.69 mmol, 2.5 eq) and Pd(PPh₃)₄ (373.60 mg, 323.30 μmol, 0.3 eq). The resulting reaction mixture was stirred at 100° C. for 1 h in sealed tube. The reaction mixture was poured onto 20 mL saturated EDTA and followed by 10 mL EtOAc. The solution was stirred at 20° C. for 0.5 h. After that, the aqueous phase was separated and extracted with EtOAc (20 mL×5). The combined organic layers were washed successively with saturated Na₂CO₃ (20 mL×1), dried over anhydrous Na₂SO₄, filtered, and concentrated to give a residue. The residue was purified by column chromatography (SiO₂, Petroleum ether/Ethyl acetate=3/1 to DCM/MeOH=10/1) which resulted in 0.25 g of a 30% purity product. Re-purity by prep-HPLC (column: Phenomenex luna C18 100×40 mm×3 μm; mobile phase: [H₂O (0.2% FA)-ACN]; gradient: 2%-25% B over 8.0 min) to give 5-chloro-2-[8-[[(3R)-1-methyl-3-piperidyl]aminolinnidazo[1,2-d][1,2,4]triazin-5-yl] phenol (42 mg, 11%) as a white solid.

¹H NMR (400 MHz, DMSO) δ 10.88 (s, 1H), 8.19 (s, 1H), 7.59 (d, 1H), 7.49 (dd, 2H), 7.25 (d, 1H), 7.14-6.97 (m, 2H), 4.32 (s, 1H), 2.87 (d, 1H), 2.57 (s, 1H), 2.24 (s, 3H), 2.21-2.04 (m, 2H), 1.87-1.68 (m, 2H), 1.58 (q, 2H).

MS: 359.1 [M+H]⁺.

### Example 8:2-(14(1s,3s)-3-Hydroxy-3-methylcyclobutoxy)pyrrolo[1,2-cl][1,2,4]triazin-4-yl)-5-(trifluoromethvflphenol

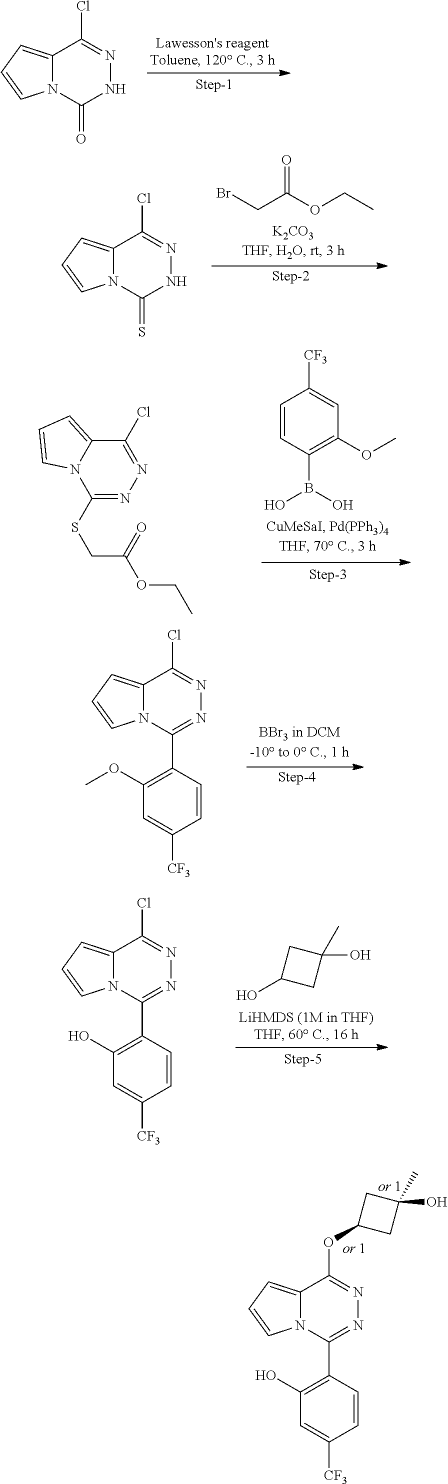

Step 1: To a stirred solution of 1-chloropyrrolo[1,2-d][1,2,4]triazin-4(3H)-one (1.0 g, 5.897 mmol) in toluene (20 mL) was added Lawesson's reagent (3.578 g, 8.846 mmol) at RT and the reaction mixture was stirred at 120° C. for 3 h. The progress of the reaction was monitored by TLC. The reaction mixture was concentrated under reduced pressure to get a crude compound as a brown gum. The crude product was purified by column chromatography using silica gel (100-200) and eluted with 0-30% EtOAc in petroleum ether as a gradient. The product was eluted at 8% EtOAc in petroleum ether. Pure compound fractions were collected and concentrated under reduced pressure to afford 1-chloropyrrolo[1,2-d][1,2,4]triazine-4(3H)-thione (0.66 g, 60%) as a white solid.

MS: 186.06 [M+H]⁺.

Step 2: To a stirred degassed solution of 1-chloropyrrolo[1,2-d][1,2,4]triazine-4(3H)-thione (600 mg, 3.232 mmol) in THF (10.0 mL) and water (5.0 mL) were added ethyl 2-bromoacetate (0.429 mL, 3.879 mmol) and potassium carbonate (670.073 mg, 4.848 mmol) at RT and the reaction mixture was stirred at RT for 3 h. The progress of the reaction was monitored by TLC. The reaction mixture was diluted with ice cold water (30 mL) and the solid was filtered and dried under vacuum to afford ethyl 2-((1-chloropyrrolo[1,2-d][1,2,4]triazin-4-yl)thio)acetate (660 mg, 75%) as an off-white solid.

MS: 272.39 [M+H]⁺.

Step 3: To a stirred degassed solution of ethyl 2-((1-chloropyrrolo[1,2-d][1,2,4]triazin-4-yl)thio)acetate (660 mg, 2.429 mmol), (2-methoxy-4-(trifluoromethyl)phenyl)boronic acid (1175.359 mg, 5.344 mmol) and copper(I) 3-methylsalicylate (1147.251 mg, 5.344 mmol) in THF (8 mL) was added tetrakis(triphenylphosphine)palladium(0) (140.344 mg, 0.121 mmol) at RT and the reaction mixture was stirred at 70° C. for 3 h. The progress of the reaction was monitored by TLC. The crude material was filtered through a celite bed and was washed with THF (10 mL). The filterate was evaporated and dried under vaccum to obtain a brown gum. The crude product was purified by column chromatography using silica gel (100-200) and eluted with 0-50% EtOAc in petroleum ether as a gradient. The product was eluted at 25% EtOAc in petroleum ether. The pure fractions were collected and concentrated under reduced pressure to afford 1-chloro-4-(2-methoxy-4-(trifluoromethyl)phenyl)pyrrolo[1,2-d][1,2,4]triazine (600 mg, 75.38%) as a yellow solid.

MS: 328.43 [M+H]⁺.

Step 4: To a stirred solution of 1-chloro-4-(2-methoxy-4-(trifluoromethyl)phenyl)pyrrolo[1,2-d][1,2,4]triazine (600 mg, 1.831 mmol) in DCM (12 mL) was added boron tribromide, 1M solution in DCM (6 mL) at −10° C. and the reaction mixture was stirred at 0° C. for 1 h. The progress of the reaction was monitored by TLC. Crude material was evaporated under reduced pressure and co-distilled with DCM (5 mL) to obtain a crude residue as a brown gum. This was further triturated with DCM (5 mL) and the resulting solid was filtered and dried thoroughly under vacuum to afford 2-(1-chloropyrrolo[1,2-d][1,2,4]triazin-4-yl)-5-(trifluoromethyl)phenol (480 mg, 83.58%) as an off-white solid.

MS: 314.08 [M+H]⁺.

Step 5: To a stirred degassed solution of 1-methylcyclobutane-1,3-diol (374.010 mg, 3.662 mmol) in THF (6 mL) was added LiHMDS 1M solution in THF (4.577 mL, 4.577 mmol) at 60° C. and the reaction mixture was stirred at 60° C. for 30 min, then cooled to RT. 1-Chloro-4-(2-methoxy-4-(trifluoromethyl)phenyl)pyrrolo[1,2-d][1,2,4]triazine (300 mg, 0.915 mmol) was added and the reaction mixture was stirred at 60° C. for 16 h. The progress of the reaction was monitored by LCMS. The reaction mixture was diluted with water (50 mL) and extracted with EtOAc (2×80 mL). The combined organic layers were separated, washed with brine solution (40 mL), dried over anhydrous sodium sulfate, filtered and concentrated under reduced pressure to get a crude product as a brown oil. The crude product was purified by column chromatography using silica gel (100-200) and eluted with 0-10% MeOH in DCM as a gradient. The product was eluted at 7% MeOH in DCM. The pure fractions were collected and concentrated under reduced pressure. The resulting residue was triturated with diethylether (5 mL), and precipitated solid was filtered and dried under vacuum to afford 2-(1-((1s,3s)-3-hydroxy-3-methylcyclobutoxy)pyrrolo[1,2-d][1,2,4]triazin-4-yl)-5-(trifluoromethyl)phenol (25 mg, 7%) as an off-white solid. ¹H NMR (400 MHz, DMSO) δ 10.94 (s, 1H), 7.70 (d, 1H), 7.38-7.29 (m, 2H), 7.22 (dd, 1H), 6.91 (dd, 1H), 6.87 (dd, 1H), 5.21 (s, 1H), 5.09 (p, 1H), 2.61 (ddd, 2H), 2.31-2.22 (m, 2H), 1.30 (s, 3H).

MS: 380.24 [M+H]⁺.

### Example 9: 5-Chloro-2-[8-[[(3R)-1-methylpyrrolidin-3-yl]amino]imidazo[1,2-d][1,2,4]triazin-5-yliphenol

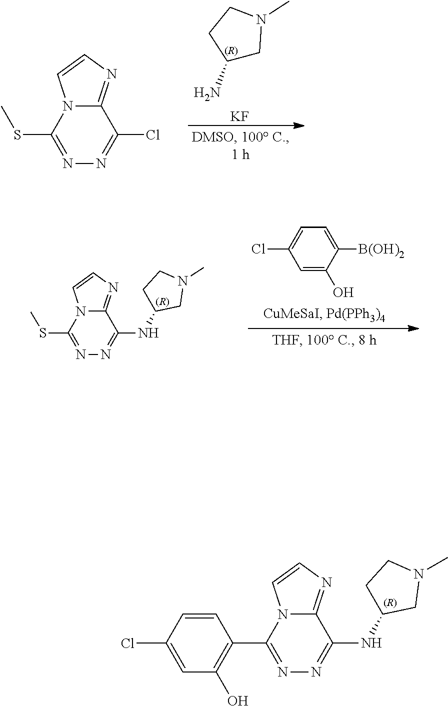

Step 1: To a solution of 8-chloro-5-methylsulfanyl-imidazo[1,2-d][1,2,4]triazine (800 mg, 3.99 mmol, 1 eq) and (3R)-1-methylpyrrolidin-3-amine (798.70 mg, 7.97 mmol, 2 eq) in DMSO (8 mL) was added KF (463.27 mg, 7.97 mmol, 186.80 μL, 2 eq). The mixture was stirred at 100° C. for 1 h. The reaction mixture was adjusted the pH to 12 with saturated Na₂CO₃ 20 mL, and then extracted with EtOAc (30 mL×3). The combined organic layers were washed with brine 20 mL, dried over Na₂SO₄, filtered and concentrated under reduced pressure to give a residue. The residue was purified by column chromatography (SiO₂, DCM:MeOH=I/O to 90/10) to afford N-[(3R)-1-methylpyrrolidin-3-yl]-5-methylsulfanyl-imidazo[1,2-d][1,2,4]triazin-8-amine (700 mg, 2.65 mmol, 66.41) as a yellow oil.

¹H NMR (400 MHz, DMSO-d6) δ=7.87 (d, 1H), 7.67 (d, 1H), 7.33 (br d, 1H), 4.56-4.46 (m, 1H), 2.82 (dd, 1H), 2.71 (s, 3H), 2.63-2.58 (m, 1H), 2.54-2.53 (m, 1H), 2.46-2.41 (m, 1H), 2.26 (s, 3H), 2.24-2.16 (m, 1H), 1.89 (tdd, 1H)

MS: 265.5 [M+H]⁺.

Step 2: To a solution of N-[(3R)-1-methylpyrrolidin-3-yl]-5-methylsulfanyl-imidazo[1,2-d][1,2,4]triazin-8-amine (300 mg, 1.13 mmol, 1 eq) and (4-chloro-2-hydroxy-phenyl)boronic acid (293.43 mg, 1.70 mmol, 1.5 eq) in THF (5 mL) was added Pd(PPh₃)₄ (393.42 mg, 340.46 μmol, 0.3 eq) and CuMeSal (609.09 mg, 2.84 mmol, 2.5 eq). The mixture was stirred at 100° C. for 8 h in a sealed tube. The reaction mixture was quenched by addition of EDTA 10 mL, and then extracted with EtOAc (10 mL×3). The combined organic layers were washed with brine 10 mL, dried over Na₂SO₄, filtered and concentrated under reduced pressure to give a residue. The residue was purified by column chromatography (SiO₂, DCM/MeOH=I/O to 5/1) and then further purified by prep-HPLC (column: Phenomenex Luna C18 100×30 mm×5 μm; mobile phase: [H₂O (0.2% FA)-ACN]; gradient: 1%-25% B over 8.0 min) to give 5-chloro-2-[8-[[(3R)-1-methylpyrrolidin-3-yl]aminolimidazo[1,2-d][1,2,4]triazin-5-yl]phenol (20.8 mg, 5%) as an off-white solid.

¹H NMR (400 MHz, DMSO-d₆) δ=11.51-10.30 (m, 1H), 7.60 (s, 2H), 7.49 (t, 2H), 7.11-7.01 (m, 2H), 4.66 (br d, 1H), 2.93 (br d, 1H), 2.73-2.61 (m, 2H), 2.55 (br d, 1H), 2.34 (br d, 3H), 2.30-2.21 (m, 1H), 2.03-1.91 (m, 1H).

¹H NMR (400 MHz, CDCl₃) δ=8.55 (br s, 1H), 7.96 (s, 1H), 7.74-7.69 (m, 2H), 7.53 (br s, 1H), 7.20 (d, 1H), 7.05-7.00 (m, 1H), 5.08 (br s, 1H), 3.53-3.47 (m, 2H), 3.10-3.05 (m, 1H), 2.74 (br d, 1H), 2.68 (s, 3H), 2.65 (br d, 1H), 2.32-2.22 (m, 1H).

MS: 345.1 [M+H]⁺.

### Example 10: 2-(8-Fluoro-1-(((1s,3s)-3-hydroxy-3-methylcyclobutyl)amino)pyrrolo[1,2-d][1,2,4]triazin-4-yl)-5-(trifluoromethyl)phenol

Analogously to the above procedure of example 8, the below example was synthesized:

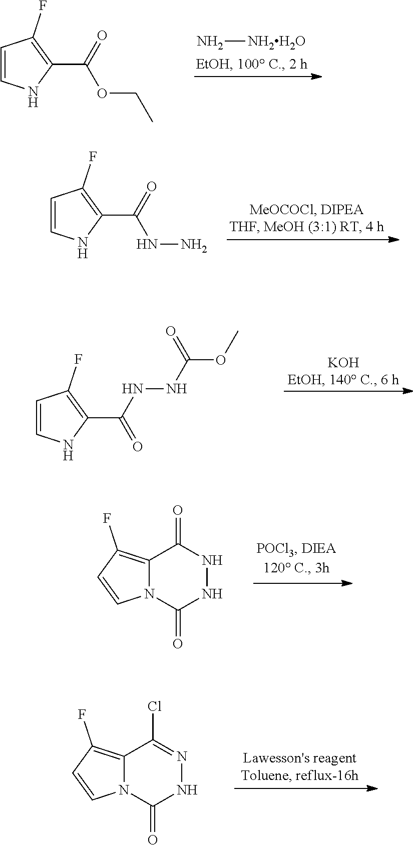

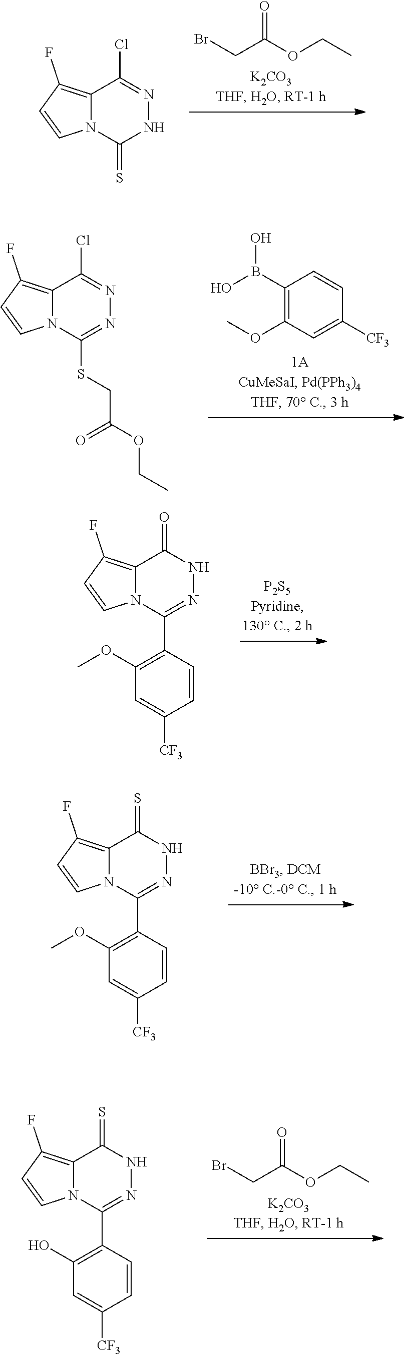

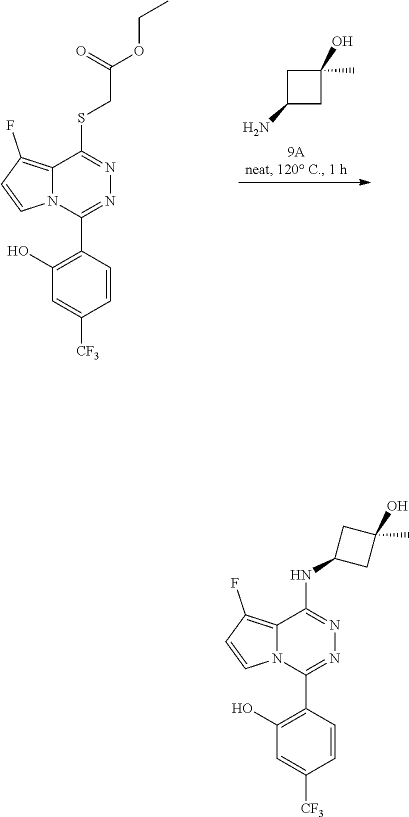

| | | | Mass |
|---|---|---|---|
| Example | IUPAC name | NMR | [M + H]⁺ |
| 10 | 2-(8-fluoro-1-(((1s,3s)-3-hydroxy-3- | ¹H NMR (400 MHz, | 397.25 |
| 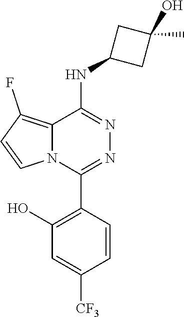 [SMILES: C[C@]1(O)C[C@@H](Nc2nnc(-c3ccc(C(F)(F)F)cc3O)n3ccc(F)c23)C1] | methylcyclobutyl)amino)pyrrolo[1,2- d][1,2,4]triazin-4-yl)-5- (trifluoromethyl)phenol | DMSO) δ 7.47 (d, 1H), 7.00 (s, 1H), 6.96 (d, 1H), 6.90 (dd, 1H), 6.62 (d, 1H), 6.44 (d, 1H), 4.94 (s, 1H), 4.14 (h, 1H), 2.42 (ddd, 2H), 2.15 (td, 2H), 1.29 (s, 3H). | |

### Example 11: 2-(4-M1s,3s)-3-Hydroxy-3-methylcyclobutyl)amino)pyrrolo[1,2-cl][1,2,4]triazin-1-yl)-5-(trifluoromethyl)phenol

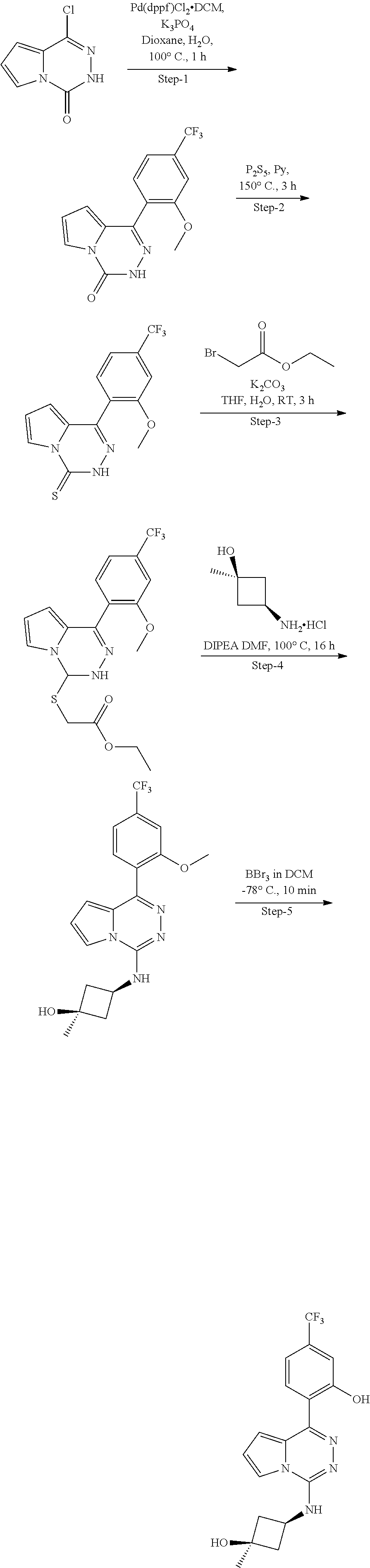

Step 1: To a stirred degassed solution of 1-chloropyrrolo[1,2-d][1,2,4]triazin-4(3H)-one (0.60 g, 3.538 mmol) in 1,4 dioxane (12 mL) and water (1.2 mL) was added (2-methoxy-4-(trifluoromethyl)phenyl)boronic acid (0.778 g, 3.538 mmol), potassium phosphate (0.751 g, 3.538 mmol) and [1,1′-bis(diphenylphosphino)ferrocene]dichloropalladium(II), complex with dichloromethane (0.144 g, 0.177 mmol) at RT and the reaction mixture was heated to 100° C. for 1 h. The progress of the reaction was monitored by TLC. The reaction mixture was quenched with ice cold water (50 mL) and stirred for 10 min. The precipitate was filtered, washed with water (50 mL) and dried under vacuum to afford 1-(2-methoxy-4-(trifluoromethyl)phenyl)pyrrolo[1,2-d][1,2,4]triazin-4(3H)-one (0.90 g, 82.25%) as an off white solid.

MS: 310.35 [M+H]⁺.

Step 2: To a stirred solution of 1-(2-methoxy-4-(trifluoromethyl)phenyl)pyrrolo[1,2-d][1,2,4]triazin-4(3H)-one (0.60 g, 1.940 mmol) in pyridine (6.0 mL) was added phosphorus pentasulfide (1.725 g, 3.880 mmol) at RT and the reaction mixture was heated to 150° C. for 3 h in a sealed tube. The progress of the reaction was monitored by TLC. The reaction mixture was diluted with ice cold water (50 mL) and stirred for 10 min. The solid which precipitated was filtered, washed with water (50 mL) and dried under vacuum to afford 1-(2-methoxy-4-(trifluoromethyl)phenyl)pyrrolo[1,2-d][1,2,4]triazine-4(3H)-thione (0.50 g, 79.22%) as an off white solid.

MS: 326.26 [M+H]⁺.

Step 3: To a stirred degassed solution of 1-(2-methoxy-4-(trifluoromethyl)phenyl)pyrrolo[1,2-d][1,2,4]triazine-4(3H)-thione (0.50 g, 1.537 mmol) in tetrahydrofuran (20.0 mL) and water (10.0 mL) was added ethyl 2-bromoacetate (0.513 g, 3.074 mmol) and potassium carbonate (1.062 g, 7.685 mmol) at RT and the reaction mixture was stirred for 3 h. The progress of the reaction was monitored by TLC. The reaction mixture was diluted with water (50 mL) and extracted with EtOAc (2×100 mL). The combined organic layers were washed with brine solution (50 mL), dried over anhydrous sodium sulfate, filtered and concentrated under reduced pressure to get a crude product as a brown oil.

MS: 412.28 [M+H]⁺.

Step 4: To a stirred solution of ethyl 2-((1-(2-methoxy-4-(trifluoromethyl)phenyl)pyrrolo[1,2-d][1,2,4]triazin-4-yl)thio)acetate (0.50 g, 1.215 mmol) and (1s,3s)-3-amino-1-methylcyclobutan-1-ol hydrochloride (0.334 g, 2.431 mmol) in N,N-dimethylformamide (10.0 mL) was added N,N-diisopropylethylamine (0.635 mL, 3.646 mmol) at RT and the reaction mixture was heated to 100° C. and stirred for 16 h in a sealed tube. The progress of the reaction was monitored by TLC. The reaction mixture was diluted with ice cold water (50 mL) and extracted with EtOAc (2×100 mL). The combined organic layers were washed with cold brine solution (50 mL), dried over anhydrous sodium sulfate, filtered and concentrated under reduced pressure to get a crude product as a brown gum. The crude product was purified by column chromatography using silica gel (100-200) and eluted with 0-10% MeOH in DCM gradient. The pure fractions were collected and concentrated under reduced pressure to afford (1s,3s)-34(1-(2-methoxy-4-(trifluoromethyl)phenyl)pyrrolo[1,2-d][1,2,4]triazin-4-yl)amino)-1-methylcyclobutan-1-ol (0.30 g, 63%) as an off white solid.

MS: 393.36 [M+H]⁺.

Step 5: To a stirred solution of (1s,3s)-34(1-(2-methoxy-4-(trifluoromethyl)phenyl)pyrrolo[1,2-d][1,2,4]triazin-4-yl)amino)-1-methylcyclobutan-1-ol (0.150 g, 0.382 mmol) in dichloromethane (7.5 mL) was added boron tribromide 1.0M in dichloromethane (1.147 mL, 1.147 mmol) at −78° C. and the reaction mixture was stirred for 10 min. The progress of the reaction was monitored by TLC. The reaction mixture was diluted with water (50 mL) and extracted with EtOAc (2×50 mL). The combined organic layers were washed with brine solution (50 mL), dried over anhydrous sodium sulfate, filtered and concentrated under reduced pressure to get a crude product as a yellow solid. The crude product was purified by Prep HPLC purification (column: Luna C18 (25×150 mm) 10 μm; buffer A: 10 mm ABC buffer B: acetonitrile mobile phase conditions (TI % B): 0/30, 2/30, 10/70, 15/70, 15.1/98, 20/98, flow: 19 mL/min, fraction volume: 200 mL). Fractions were collected and concentrated under reduced pressure and lyophilized to afford 2-(4-(((1s,3s)-3-hydroxy-3-methylcyclobutyl)-amino)pyrrolo[1,2-d][1,2,4]triazin-1-yl)-5-(trifluoromethyl)phenol (0.047 g, 32%) as an off white solid. ¹H NMR (400 MHz, DMSO) δ 13.60 (s, 1H), 8.14 (d, 1H), 8.09 (d, 1H), 8.06-7.90 (m, 1H), 7.36-7.24 (m, 2H), 7.21 (d, 1H), 7.14-7.06 (m, 1H), 5.08 (s, 1H), 4.30-4.13 (m, 1H), 2.49-2.46 (m, 2H), 2.21 (dd, 2H), 1.34 (s, 3H).

MS: 379.25 [M+H]⁺.

### Example 12: 2-(1-(((1s,3s)-3-Hydroxy-3-methylcyclobutyl)amino)pyrrolo[1,2-cl][1,2,4]triazin-4-yl)-5-(trifluoromethyl)phenol

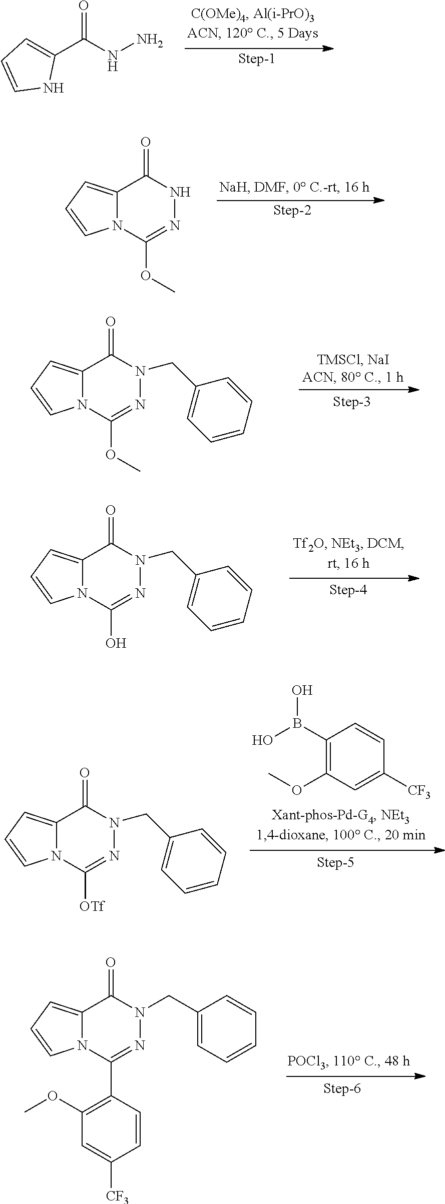

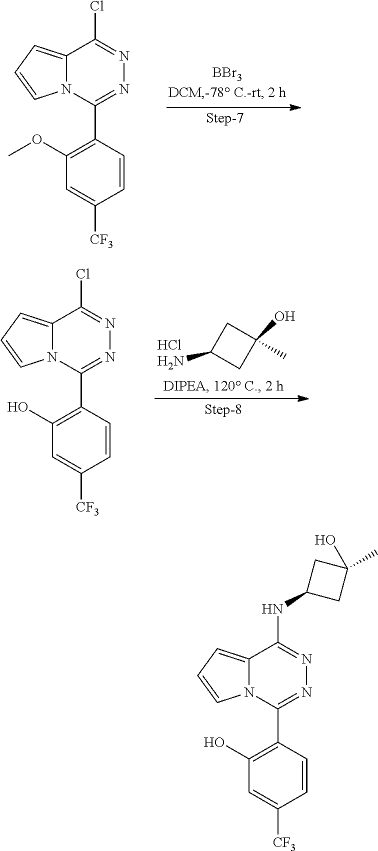

Step 1: To a stirred solution of 1H-pyrrole-2-carbohydrazide (15 g, 119.874 mmol) and tetramethyl orthocarbonate (24.239 mL, 179.812 mmol) in acetonitrile (250 mL) was added aluminium isopropoxide (4.897 g, 23.975 mmol) at RT and the reaction mixture was stirred at 120° C. for 5 days in a sealed tube. The progress of the reaction was monitored by TLC. The reaction mixture was cooled to RT and diluted with ACN (40 mL), the formed precipitate was filtered and dried throughly under vaccum to afford 4-methoxypyrrolo[1,2-d][1,2,4]triazin-1(2H)-one (7.5 g, 38%) as a yellow solid.

MS: 166.31 [M+H]⁺.

Step 2: To a stirred solution of sodium hydride, 60% (3.270 g, 136.238 mmol) in N,N-dimethylformamide (60 mL) was added 4-rnethoxypyrrolo[1,2-d][1,2,4]triazin1(2H)-one (7.5 g, 45.413 mmol) at 0° C., the reaction mixture was stirred for 10 min, then benzyl bromide (6.473 mL, 54.495 mmol) was added at 0° C. The resulting mixture was stirred at RT for 16 h. The progress of the reaction was monitored by TLC. The reaction mixture was quenched with ice cold water (120 mL) and stirred for 30 min. The solid which precipitated was filtered, washed with water (10 mL) and dried under vacuum to afford 2-benzyl-4-methoxypyrrolo[1,2-d][1,2,4]triazin-1(2H)-one (6 g, 55%) as an off-white solid.

MS: 256.11 [M+H]⁺.

Step 3: To a stirred solution of 2-benzyl-4-methoxypyrrolo[1,2-d][1,2,4]triazin-1(2H)-one (6 g, 23.504 mmol) in acetonitrile (120 mL) were added chlorotrimethylsilane (6.563 mL, 51.709 mmol) and sodium iodide (7.751 g, 51.709 mmol) at RT and the reaction mixture was stirred at 80° C. for 1 h. The progress of the reaction was monitored by TLC. The reaction mixture was quenched with ice cold water (150 mL) and stirred for 30 min. The solid was precipitated, filtered, washed with water (20 mL) and dried under vacuum to afford 2-benzyl-4-hydroxypyrrolo[1,2-d][1,2,4]triazin-1(2H)-one (5 g, 88%) as an off white solid.

MS: 242.07 [M+H]⁺.

Step 4: To a stirred solution of 2-benzyl-4-hydroxypyrrolo[1,2-d][1,2,4]triazin-1(2H)-one (2.0 g, 8.290 mmol) in DCM (200 mL) were added triethylamine (2.311 mL, 16.580 mmol) and trifluoromethanesulfonic anhydride (2.784 mL, 16.580 mmol) dropwise at 0° C. and the reaction mixture was stirred at RT for 16 h. The progress of the reaction was monitored by TLC. The reaction mixture was diluted with water (60 mL) and extracted with DCM (2×60 ml). The combined organic layers were dried over Na₂SO₄, filtered, and evaporated under reduced pressure to get a crude compound as brown gum. The crude compound was purified by column chromatography using silica gel (100-200) and eluted with DCM as a gradient. The product was eluted in 100% DCM. The pure fractions were collected and concentrated under reduced pressure to afford 2-benzyl-1-oxo-1,2-dihydropyrrolo[1,2-d][1,2,4]triazin-4-yl trifluoromethanesulfonate (1.9 g, 61%) as an off white solid.

MS: 374.36 [M+H]⁺.

Step 5: To a degassed solution of (2-methoxy-4-(trifluoromethyl)phenyl)boronic acid (1.679 g, 7.634 mmol) and triethylamine (2.128 mL, 15.269 mmol) in 1,4-dioxane (10 mL) was added Xantphos Pd G4 (0.490 g, 0.509 mmol) at RT and the reaction mixture was stirred at 100° C. for 3 min. Then 2-benzyl-1-oxo-1,2-dihydropyrrolo[1,2-d][1,2,4]triazin-4-yl trifluoromethanesulfonate (1.9 g, 5.090 mmol) in 1,4-dioxane (10 mL) was added dropwise for 20 min and the reaction mixture was stirred for 20 min at the same temperature. The progress of the reaction was monitored by TLC. The reaction mixture was diluted with water (30 mL) and extracted with EtOAc (2×70 ml). The combined organic layers were dried over Na₂SO₄, filtered, and evaporated under reduced pressure to get a crude compound as a brown gum. The crude product was purified by column chromatography using silica gel (100-200) and eluted with EtOAc in petroleum ether as a gradient. The product was eluted at 20% EtOAc in petroleum ether. The pure fractions were collected and concentrated under reduced pressure to afford 2-benzyl-4-(2-methoxy-4-(trifluoromethyl)phenyl)pyrrolo[1,2-d][1,2,4]triazin-1(2H)-one (1.5 g, 73.79%) as an off white solid.

MS: 400.20 [M+H]⁺.

Step 6: To the compound 2-benzyl-4-(2-methoxy-4-(trifluoromethyl)phenyl)pyrrolo[1,2-d][1,2,4]triazin-1(2H)-one (500 mg, 1.252 mmol) was added phosphoryl chloride (6 mL) dropwise at 0° C. and the reaction mixture was stirred at 110° C. for 48 h. The progress of the reaction was monitored by LCMS. Crude material was evaporated under reduced pressure. The reaction mixture was quenched with saturated ice cold bicarbonate solution (12 mL) and extracted with EtOAc (2×30 mL). The combined organic layers were washed with brine solution (20 mL), dried over anhydrous sodium sulfhate, filtered and concentrated under reduced pressure to get a crude product as a brown gum. The crude product was purified by column chromatography using silica gel (100-200) and eluted with EtOAc in petroleum ether as a gradient. The product was eluted at 20% EtOAc in petroleum ether. The pure fractions were collected and concentrated under reduced pressure to afford 1-chloro-4-(2-methoxy-4-(trifluoromethyl)phenyl)pyrrolo[1,2-d][1,2,4]triazine (60 mg, 14.62%) as an off-white solid.

MS: 328.10 [M+H]⁺.

Step 7: To a stirred solution of 1-chloro-4-(2-methoxy-4-(trifluoromethyl)phenyl)pyrrolo[1,2-d][1,2,4]triazine (40 mg, 0.122 mmol) in DCM (4 mL) was added boron tribromide 1M in dichloromethane (0.366 mL, 0.366 mmol) at −78° C. and the reaction mixture was warmed slowly to RT and stirred for 2 h. The progress of the reaction was monitored by LCMS. The reaction mixture was diluted with water (10 mL) and extracted with EtOAc (2×30 mL). The combined organic layers were washed with brine solution (20 mL), dried over anhydrous sodium sulfate, filtered and concentrated under reduced pressure to get a crude product as an off-white gum.

MS: 314.12 [M+H]⁺.

Step 8: To a stirred solution of 2-(1-chloropyrrolo[1,2-d][1,2,4]triazin-4-yl)-5-(trifluoromethyl)phenol (45 mg, 0.143 mmol) in N,N-diisopropylethylamine (0.5 mL) was added (1s,3s)-3-amino-1-methylcyclobutan-1-ol hydrochloride (23.690 mg, 0.172 mmol) in a sealed tube and the reaction mixture was stirred at 120° C. for 3 h. The progress of the reaction was monitored by LCMS. The reaction mixture was evaporated under reduced pressure to get a crude residue as a yellow solid. This product was purified by Prep HPLC purification (mobile phase: 10 mM ammonium bicarbonate (Aq): MeCN, column: X select-CSH, flow 5 ml/min, gradient method: 0/30, 10/60, 10.01/100, 16/100, 16.01, 30, 21/30). Fractions were collected, concentrated under reduced pressure and lyophilized to afford 2-(1-(((1s,3s)-3-hydroxy-3-methylcyclobutyl)amino)pyrrolo[1,2-d][1,2,4]triazin-4-yl)-5-(trifluoromethyl)phenol (5 mg, 9.2%) as an off white solid. ¹H NMR (400 MHz, DMSO) δ 10.82 (s, 1H), 7.64 (d, 1H), 7.43 (d, 1H), 7.38-7.24 (m, 2H), 7.14-6.99 (m, 2H), 6.71 (dd, 1H), 5.01 (s, 1H), 4.15 (h, 1H), 2.47-2.39 (m, 2H), 2.22-2.04 (m, 2H), 1.31 (s, 3H).

MS: 379.30 [M+H]⁺.

### Example 13: 2-(4-((1s,3s)-3-Hydroxy-3-methylcyclobutoxy)pyrrolo[1,2-d][1,2,4]triazin-1-yl)-5-(trifluoromethyl)phenol

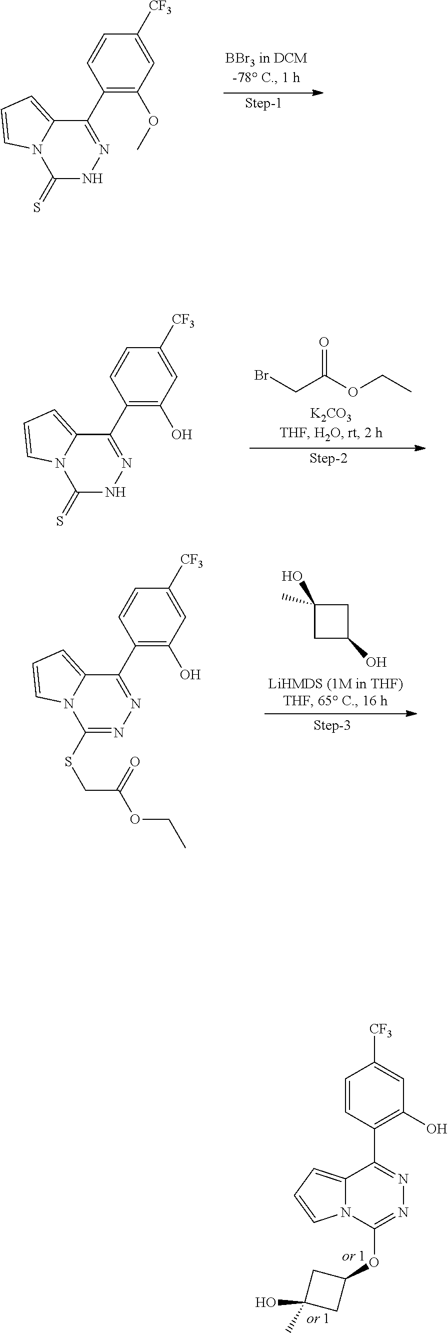

Step 1: To a stirred solution of 1-(2-methoxy-4-(trifluoromethyl)phenyOpyrrolo[1,2-d][1,2,4]triazine-4(3H)-thione (0.30 g, 0.922 mmol) in DCM (15.0 mL) was added boron tribromide 1.0M in DCM (2.767 mL, 2.767 mmol) at −78° C. and the reaction mixture was stirred for 1 h. The progress of the reaction was monitored by TLC. The reaction mixture was diluted with water (100 mL) and extracted with EtOAc (2×100 mL). The combined organic layers were washed with brine solution (50 mL), dried over anhydrous sodium sulfate, filtered and concentrated under reduced pressure to get a crude product as a pale yellow solid.

MS: 312.33[M+H]⁺.

Step 2: To a stirred degassed solution of 1-(2-hydroxy-4-(trifluoromethyl)phenyl)pyrrolo[1,2-d][1,2,4]triazine-4(3H)-thione (0.25 g, 0.803 mmol) in THF (10.0 mL) and water (5.0 mL) was added ethyl 2-bromoacetate (0.134 g, 0.803 mmol) and potassium carbonate (0.166 g, 1.205 mmol) at RT and the reaction mixture was stirred for 2 h. The progress of the reaction was monitored by TLC. The reaction mixture was diluted with water (100 mL) and extracted with EtOAc (2×200 mL). The combined organic layers were washed with brine solution (50 mL), dried over anhydrous sodium sulfate, filtered and concentrated under reduced pressure to get a crude product as a yellow solid.

MS: 398.23 [M+H]⁺.

Step 3: To a stirred solution of 1-methylcyclobutane-1,3-diol (0.964 g, 9.437 mmol) in THF (6.0 mL) was added LiHMDS 1M solution in THF (3.146 mL, 3.146 mmol) in one charge at RT and the reaction mixture was heated to 65° C. for 10 min. Then, ethyl 2-((1-(2-hydroxy-4-(trifluoromethyl)phenyl)pyrrolo[1,2-d][1,2,4]triazin-4-yl)thio)acetate (0.250 g, 0.629 mmol) in THF (4.0 mL) was added to the suspension at RT and the reaction mixture was heated to 65° C. and stirred for 16 h in a sealed tube. The progress of the reaction was monitored by TLC. The reaction mixture was diluted with water (100 mL) and extracted with EtOAc (2×200 mL). The combined organic layers were washed with brine solution (50 mL), dried over anhydrous sodium sulfate, filtered and concentrated under reduced pressure to get a crude product as a brown gum. The crude product was purified by Prep HPLC purification. (buffer A: 10 mm ABC, buffer B: ACN, mobile phase conditions (% of B): 0/40, 2/40, 10/70, 16/70, 16.01/9, column: inersil flow-19 mL/min). Pure fractions were collected and concentrated under reduced pressure and lyophilized to afford 2-(4-((1s,3s)-3-hydroxy-3-methylcyclobutoxy)pyrrolo[1,2-d][1,2,4]triazin-1-yl)-5-(trifluorornethyl)phenol (0.017 g, 7%) as an off white solid.

¹H NMR (400 MHz, DMSO) δ 12.23 (s, 1H), 7.84 (d, 1H), 7.77 (dd, 1H), 7.25 (s, 1H), 7.20 (d, 1H), 7.05 (dd, 1H), 6.96 (dd, 1H), 5.46-5.10 (m, 2H), 2.74-2.61 (m, 2H), 2.44-2.33 (m, 2H), 1.32 (s, 3H).

MS: 380.26 [M+H]⁺.

### Example 14: 2-[8-[[(3R)-1-Ethyl-3-piperidy]iamino]imidazo[1,2-d][1,2,4]triazin-5-yl]-5-(trifluoromethyl)phenol

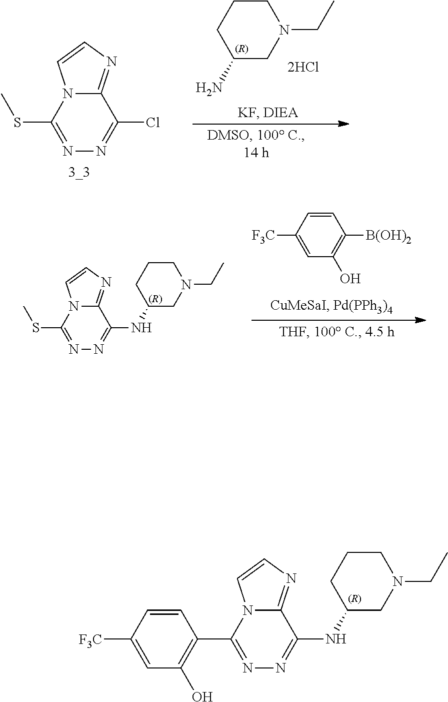

Step 1: A mixture of 8-chloro-5-methylsulfanyl-imidazo[1,2-d][1,2,4]triazine (0.5 g, 2.49 mmol, 1 eq), (3R)-1-ethylpiperidin-3-amine (1.00 g, 4.98 mmol, 2 eq, 2HCl), DIEA (1.29 g, 9.97 mmol, 1.74 mL, 4 eq), KF (289.54 mg, 4.98 mmol, 116.75 μL, 2 eq) in DMSO (5 mL) was degassed and purged with N₂ for 3 times, and then the mixture was stirred at 100° C. for 14 h under N₂ atmosphere. The reaction mixture was quenched by addition H₂O (50 mL) and then extracted with EtOAc (30 mL×3). The combined organic layers were washed with brine (50 mL×3), dried over Na₂SO₄, filtered and concentrated under reduced pressure to give a residue. The residue was purified by column chromatography (SiO₂, Petroleum ether/Ethyl acetate=4/1 to 1/1 and then DCM:MeOH:TEA=40:2:1 to DCM:MeOH:TEA=20:2:1, TLC (PE:EtOAc=1:1, R_{f}=0, DCM:MeOH:TEA=20:2:1, R_{f}=0.2). The purified product was diluted with EtOAc 50 mL and then washed with brine 150 mL (50 mL*3) to afford the title compound N-[(3R)-1-ethyl-3-piperidyl]-5-methylsulfanyl-imidazo[1,2-d][1,2,4]triazin-8-amine (470 mg, 43%) as a yellow oil. ¹H NMR (400 MHz, DMSO-d6) δ=7.90-7.85 (m, 1H), 7.67 (d, J=1.3 Hz, 1H), 7.14-6.95 (m, 1H), 4.25-4.13 (m, 1H), 2.93-2.85 (m, 1H), 2.71 (s, 3H), 2.66-2.60 (m, 1H), 2.43-2.33 (m, 2H), 2.21-2.03 (m, 2H), 1.84-1.74 (m, 1H), 1.72-1.64 (m, 1H), 1.62-1.46 (m, 2H), 1.05-0.98 (m, 3H).

MS: 293.0 [M+H]⁺.

Step 2: A mixture of N-[(3R)-1-ethyl-3-piperidyl]-5-methylsulfanyl-imidazo[1,2-d][1,2,4]triazin-8-amine (0.18 g, 615.59 μmol, 1 eq), [2-hydroxy-4-(trifluoromethyl)phenyl]boronic acid (253.53 mg, 1.23 mmol, 2 eq), cuprous; 2-hydroxy-3-methyl-benzoate (660.79 mg, 3.08 mmol, 5 eq) and Pd(PPh₃)₄ (426.81 mg, 369.35 μmol, 0.6 eq) in THF (10 mL) was degassed and purged with N₂ for 3 times, and then the mixture was stirred at 100° C. for 4.5 h under N₂ atmosphere. The reaction mixture was poured into EDTA (50 mL) and stirred at room temperature for 1 h. The mixture was extracted with EtOAc (50 mL×3). The combined organic layers were washed with brine (50 mL×3), dried over Na₂SO₄, filtered and concentrated under reduced pressure to give a residue. The residue was purified by column chromatography (SiO₂, Petroleum ether/Ethyl acetate=2/1 to 1/2 and then DCM:MeOH:TEA=20:2:1, TLC (PE:EtOAc=1:1, R_{f}=0, DCM:MeOH:TEA=20:2:1, R_{f}=0.2)) and then purified by column chromatography (SiO₂, Petroleum ether/Ethyl acetate=2/1 to 1/2 and then DCM:MeOH:TEA=20:2:1, TLC (PE:EtOAc=1:1, R_{f}=0, DCM:MeOH:TEA=20:2:1, R_{f}=0.2)) and then purified by prep-HPLC (column: Waters Xbridge Prep OBD C18 150*40 mm*10 um; mobile phase: [H₂O (10 mM NH₄HCO₃)-ACN]; gradient: 15%-45% B over 8.0 min) to afford 2[8-[[(3R)-1-ethyl-3-piperidyl]anninolimidazo[1,2-d][1,2,4]triazin-5-yl]-5-(trifluoromethyl)phenol (36.75 mg, 14%) as a white solid.

LC-MS(ES+, m/z): 407.1 [(M+H)⁺].

¹H NMR (400 MHz, DMSO-d₆) δ=11.41-10.70 (m, 1H), 7.77-7.66 (m, 1H), 7.64-7.57 (m, 1H), 7.56-7.49 (m, 1H), 7.39-7.20 (m, 3H), 4.44-4.22 (m, 1H), 2.99-2.83 (m, 1H), 2.74-2.57 (m, 1H), 2.39 (q, 2H), 2.25-2.02 (m, 2H), 1.88-1.79 (m, 1H), 1.77-1.46 (m, 3H), 1.02 (t, 3H).

MS: 407.1 [M+H]⁺.

### Example 15: 5-Chloro-2-[8-[[(3R)-1-ethyl-3-piperidyl]amino]imidazo[1,2-d][1,2,4]triazin-5-yliphenol

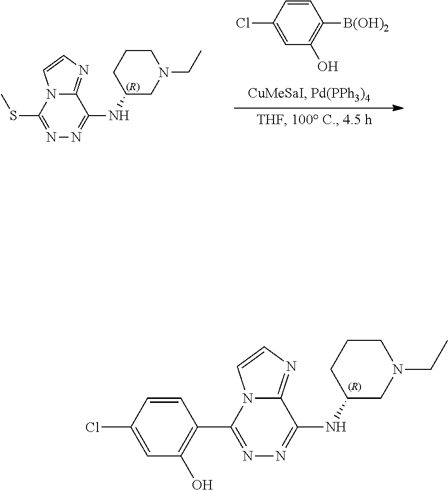

A mixture of N-[(3R)-1-ethyl-3-piperidyl]-5-methylsulfanyl-imidazo[1,2-d][1,2,4]triazin-8-amine (0.18 g, 615.59 μmol, 1 eq), (4-chloro-2-hydroxy-phenyl)boronic acid (212.22 mg, 1.23 mmol, 2 eq), cuprous; 2-hydroxy-3-methyl-benzoate (660.79 mg, 3.08 mmol, 5 eq) and Pd(PPh₃)₄ (426.81 mg, 369.35 μmol, 0.6 eq) in THF (10 mL) was degassed and purged with N₂ for 3 times, and then the mixture was stirred at 100° C. for 4.5 h in sealed tube under N₂ atmosphere. The reaction mixture was poured into EDTA (50 mL) and stirred at room temperature for 1 h. The mixture was extracted with EtOAc (50 mL×3). The combined organic layers were washed with brine (50 mL×3), dried over Na₂SO₄, filtered and concentrated under reduced pressure to give a residue. The residue was purified by column chromatography (SiO₂, Petroleum ether/Ethyl acetate=2/1 to 1/2 and then DCM:MeOH:TEA=20:2:1, TLC (PE:EtOAc=1:1, R_{f}=0, DCM:MeOH:TEA=20:2:1, R_{f}=0.2)) and further purified by prep-HPLC (column: Waters Xbridge Prep OBD C18 150*40 mm*10 um; mobile phase: [H₂O(10 mM NH₄HCO₃)-ACN]; gradient: 15%-45% B over 8.0 min) to afford 5-chloro-2-[8-[[(3R)-1-ethyl-3-piperidyl]amino]imidazo[1,2-d][1,2,4]triazin-5-yliphenol (42.27 mg, 18%) as a white solid.

¹H NMR (400 MHz, DMSO-d₆) δ=11.30-10.15 (m, 1H), 7.66-7.56 (m, 1H), 7.50 (br s, 2H), 7.32-7.15 (m, 1H), 7.09-6.97 (m, 2H), 4.37-4.19 (m, 1H), 2.98-2.82 (m, 1H), 2.71-2.56 (m, 1H), 2.42-2.33 (m, 2H), 2.24-2.01 (m, 2H), 1.88-1.77 (m, 1H), 1.76-1.45 (m, 3H), 1.01 (t, 3H).

MS: 373.1 [M+H]⁺.

Biological Assay Description

1.1 NLRP3 Inhibition Assay

The following assays were used to determine the inhibitory activity of test compounds on the NLRP3 inflammasome pathway using common stimuli Nigericin (Invivogen) or monosodium urate crystals (MSU) (Invivogen).

Cell Culture

Human monocyte-like cells were cultured in RPMI-1640 Glutamax medium supplemented with 10% heat inactivated FBS (Fetal Bovine Serum) and 50 U/ml penicillin-streptomycin (Life Technologies).

NLRP3 Inflammasome Pathway Activation Assay

Human monocyte-like cells were seeded at 25000 per well in a 384-well plate and were differentiated overnight into macrophages with 10 ng/ml PMA (Phorbol Myristate Acetate). The following day, medium containing 10 ng/ml LPS (Lipopolysaccharide) was added. After 3 hours of LPS priming, concentrations of test compound in the range from 100 μM to 380 μM were added 30 min prior to NLRP3 inflammasome pathway stimulation with Nigericin 3.75 μM or MSU 200 μg/ml for 3 h.

Measurement of IL-1β

For IL-1β quantification, supernatants were analyzed using HTRF kit according to the manufacturer's instructions (Cisbio 62HIL1BPEH), Briefly, in a 384-well ProxiPlate™ microplate, 8 μl of sample was mixed, with 5 μl of Anti-IL1β Cryptate antibody (40×) and Anti-IL1β XL antibody (40×). Then, incubated overnight at RT. Reading was done using an EnVision Reader (PerkinElmer).

IC₅₀ (concentration corresponding to 50% inhibition) were determined using GraphPad Prism 9.

The following example compounds were measured:

TABLE 1

| | IC₅₀ Human | IC₅₀ Human |
|---|---|---|
| | monocyte-like cells | monocyte-like cells |
| Examples | MSU (nM) | Nigericin (nM) |
| 1 | ++++ | ++++ |
| 2 | +++ | ND |
| 3 | +++ | ND |
| 4 | ++ | ++ |
| 5 | ++++ | +++ |
| 6 | ++ | ND |
| 7 | ++++ | ND |
| 8 | ++++ | +++ |
| 9 | ++ | ND |
| 10 | ++++ | ND |
| 11 | ++++ | +++ |
| 12 | +++ | ++ |
| 13 | +++ | ++ |
| 14 | ++++ | ND |
| 15 | ++++ | ND |

Legend:
++++ IC₅₀ ≤5 nM;
+++ IC₅₀ 5 nM < X < 20 nM;
++ IC₅₀ 20 nM < X < 100 nM;
+ IC₅₀ >100 nM;
ND not determined.

The tested compounds showed inhibition of IL-1 beta release in human monocyte-like cells using MSU or Nigericin as activators, see Table 1.

The tested compounds of the present invention 8 and 11 with Z₁=N and Z₂=C surprisingly showed significantly increased inhibition of IL-1 beta release in human monocyte-like cells using MSU or Nigericin as activators, see Table 1, compared to the equivalent compounds 13 and 12, respectively, in which Z₁=C and Z₂=N.

Results demonstrate that compounds of the present invention with Z₁=N and Z₂=C surprisingly show significantly increased inhibition of IL-1 beta release in human monocyte-like cells using MSU or Nigericin as activators, compared to the equivalent compounds in which Z₁=C and Z₂=N.

1.2 Inhibition of IL-1Beta (IL-1β) in an LPS/ATP Induced Acute Peritonitis Mouse Model

The efficacy of compound 5 in an LPS/ATP-induced acute peritonitis was evaluated: LPS (Lipopolysaccharide) and ATP (Adenosine triphosphate) were administered to BALB/c mice by intraperitoneal (i.p.) injection to elicit an acute inflammatory response in the peritoneal cavity.

Method

Mice (BALB/c mice, aged 6-8 w, females) were challenged with 50 μg/kg LPS by intraperitoneal (i.p.) injection. 120 min after the injection of LPS, ATP was intraperitoneally (i.p.) injected at 50 mM in 200 μL. Compound 5 was formulated at a dosing volume of 10 mL/kg in 0.5% carboxymethylcellulose sodium salt with 0.2% Tween 80 in water. Compound 5 was dosed 30 min before LPS injection. 30 min post ATP injection, animals were sacrificed by CO₂. For the peritoneal wash (PW) collection 3 mL cold phosphate-buffered saline (PBS)/heparin (25 U per mL) was injected in the peritoneal cavity using a 25 G needle and the abdominal area was gently massaged for 2 min. Lavage fluid was collected, and supernatant was harvested after centrifugation (300×g, for 10 min at 4° C.). The volume of the supernatant was reported, and aliquoted into three Eppendorf tubes of 250 μL each. Tubes were snap frozen on dry ice.

Cytometric Bead Array (CBA) Assay for IL-1beta detection was performed on the PW supernatant as follows: supernatants or cytokine standards were incubated with the capture beads mixture for 1 hour at room temperature. Dilution factor in peritoneal lavage: for TNF-α and IL-1b, samples were diluted 2-fold; for IL-6, samples were diluted 20-fold. The capture beads were then incubated with the phycoerythrin (PE) Detection Reagents mixture containing PE-conjugated anti-mouse IL-113, TNF, and IL-6 for 1 hour at room temperature. After washing in Wash Buffer containing 1x PBS, blocking protein, and detergent, the beads were re-suspended and analyzed by flow cytometry.

Data was exported and plotted using GraphPad Prism 7. The cytokine analysis raw data were analyzed using FACP. Data were expressed as the Mean±SD. One-way ANOVA was used to compare significance among groups.

Results

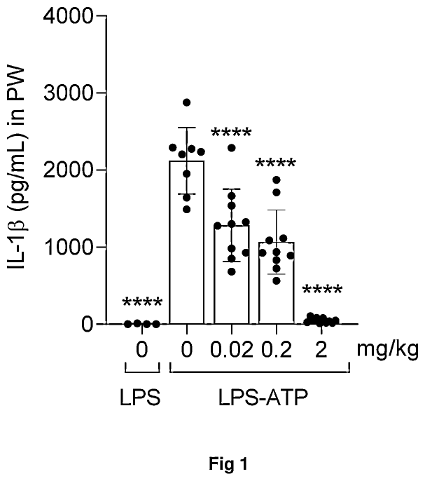

Mice challenged with LPS and ATP showed increased IL-1beta levels in plasma and in peritoneal wash (PW), indicating successful model induction. As seen from FIG. 1, compound 5 significantly reduced IL-18 release in PW in a dose responsive manner in an LPS-ATP acute peritonitis model (n=8, One-way ANOVA with Dunnett's multiplicity adjustment. Comparisons against group: LPS-ATP, 0 mg/kg. ****p>0.0001). It was further observed that compound 5 exhibited no inhibition on either IL-6 or TNF-α in plasma or PW (data not shown).

1. A compound of formula (I) 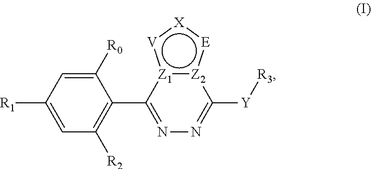
or a stereoisomer, a racemic mixture, a tautomer, a polymorph, a pharmaceutically acceptable salt, a prodrug, a hydrate, or a solvate thereof;
wherein
Z₁ and Z₂ are each selected from N and C whereby if Z₁ is N, Z₂ is C, and if Z₁ is C, Z₂ is N;
V, X and E are each independently selected from N and CRₐ;
wherein at least one of V, X and E is CRₐ;
Rₐ is independently selected from the group consisting of —H, -C₁-C₃alkyl, —CF₃ and halo;
R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH and O; and
R₃ is selected from the group consisting of
4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄alkyl, haloC₁-C₄alkyl, hydroxyC₁-C₄alkyl, —OH and halo;
5- or 6-membered aryl or heteroaryl, wherein heteroaryl contains one or two heteroatoms, wherein said heteroatom(s) is/are N, optionally substituted with one or two substituents independently selected from the group consisting of C₁-C₄alkyl, hydroxyC₁-C₄alkyl, haloC₁-C₄alkyl, —CN and halo;
C₃-C₆cycloalkyl optionally substituted with one or two substituents independently selected from the group consisting of C₁-C₄alkyl, haloC₁-C₄alkyl, hydroxyC₁-C₄alkyl, —OH and halo; and
C₁-C₆alkyl substituted with one or two substituents independently selected from the group consisting of —OH, halo, haloC₁-C₄alkyl and C₁-C₄alkoxy.

2. A compound of formula (I′) 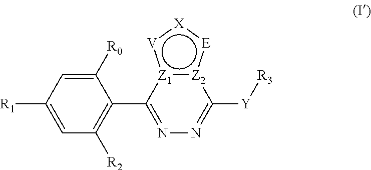
or a stereoisomer, a racemic mixture, a tautomer, a polymorph, a pharmaceutically acceptable salt, a prodrug, a hydrate, or a solvate thereof;
wherein
Z₁ is N;
Z₂ is C;
V, X and E are each independently selected from N and CRₐ;
wherein at least one of V, X and E is CRₐ;
Rₐ is independently selected from the group consisting of —H, -C₁-C₃alkyl, —CF₃ and halo;
R₀ is selected from the group consisting of —H, C₁-C₃alkyl and halo;
R₁ is selected from the group consisting of —CF₃, —OCF₃, —OCHF₂ and halo;
R₂ is selected from the group consisting of —OH, —H and —CF₃;
Y is selected from NH and O; and
R₃ is selected from the group consisting of
4-, 5- or 6-membered heterocycloalkyl containing one or two heteroatoms, preferably one heteroatom, wherein said heteroatom(s) is/are N, optionally substituted with one or two substituents independently selected from the group consisting of C₁-C₄alkyl, haloC₁-C₄alkyl, hydroxyC₁-C₄alkyl, —OH and halo;
5- or 6-membered aryl or heteroaryl, wherein heteroaryl contains one or two heteroatoms, wherein said heteroatom(s) is/are N, optionally substituted with one or two substituents independently selected from the group consisting of C₁-C₄alkyl, hydroxyC₁-C₄alkyl, haloC₁-C₄alkyl, —CN and halo;
C₃-C₆ cycloalkyl optionally substituted with one or two substituents independently selected from the group consisting of C₁-C₄alkyl, haloC₁-C₄alkyl, hydroxyC₁-C₄alkyl, —OH and halo; and
C₁-C₆ alkyl substituted with one or two substituents independently selected from the group consisting of —OH, halo, haloC₁-C₄alkyl and C₁-C₄alkoxy.

3. The compound according to claim 1, having the formula (Ib) 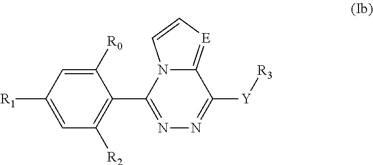
or a stereoisomer, a racemic mixture, a tautomer, a polymorph, a pharmaceutically acceptable salt, a prodrug, a hydrate, or a solvate thereof;
wherein R₀, R₁, R₂, R₃, E and Y are as defined for formula (I).

4. The compound according to claim 1, having the formula (Ic) 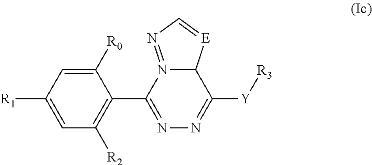
or a stereoisomer, a racemic mixture, a tautomer, a polymorph, a pharmaceutically acceptable salt, a prodrug, a hydrate, or a solvate thereof;
wherein R₀, R₁, R₂, R₃, E and Y are as defined for formula (I), preferably E is CRₐ
wherein CRₐ is as defined for formula (I).

5. The compound according to claim 1, having the formula (Ie) 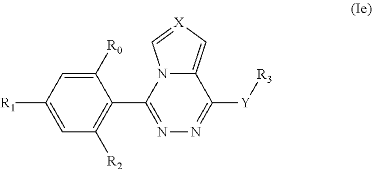
or a stereoisomer, a racemic mixture, a tautomer, a polymorph, a pharmaceutically acceptable salt, a prodrug, a hydrate, or a solvate thereof;
wherein
X is selected from N and CRₐ;
Rₐ is selected from selected from the group consisting of -C₁-C₃alkyl, —CF₃ and halo; and
R₀, R₁, R₂, R₃ and Y are as defined for formula (I).

6. The compound according to claim 1, having the formula (Ia) 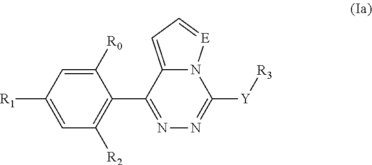
or a stereoisomer, a racemic mixture, a tautomer, a polymorph, a pharmaceutically acceptable salt, a prodrug, a hydrate, or a solvate thereof;
wherein
E is selected from N and CRₐ;
Rₐ is selected from the group consisting of —H, -C₁-C₃alkyl and —CF₃;
R₀, R₁, R₂, R₃, and Y are as defined for formula (I).

7. The compound according to claim 1, having the formula (Id) 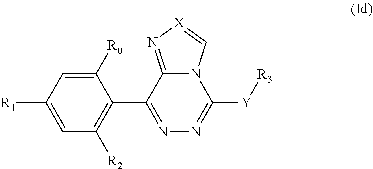
or a stereoisomer, a racemic mixture, a tautomer, a polymorph, a pharmaceutically acceptable salt, a prodrug, a hydrate, or a solvate thereof;
wherein R₀, R₁, R₂, R₃, X and Y are as defined for formula (I), preferably X is CRₐ
wherein CRₐ is as defined for formula (I).

8. The compound of formula I, I′, Ia, Ib, Ic, Id or Ie according to claim 1, wherein
R₃ is selected from the group consisting of
4-, 5- or 6-membered heterocycloalkyl containing one heteroatom, wherein said heteroatom is N, optionally substituted with one or two substituents independently selected from the group consisting of C₁-C₄alkyl;
5- or 6-membered heteroaryl, wherein heteroaryl contains one or two heteroatoms, wherein said heteroatom(s) is/are N, optionally substituted with one or two substituents independently selected from the group consisting of C₁-C₄alkyl;
C₃-C₆ cycloalkyl optionally substituted with one or two substituents independently selected from the group consisting of C₁-C₄alkyl, —OH and halo; and
hydroxyC₁-C₆ alkyl.

9. The compound of formula I, I′, Ia, Ib, Ic, Id or Ie according to claim 1, wherein R₃ is selected from the group consisting of 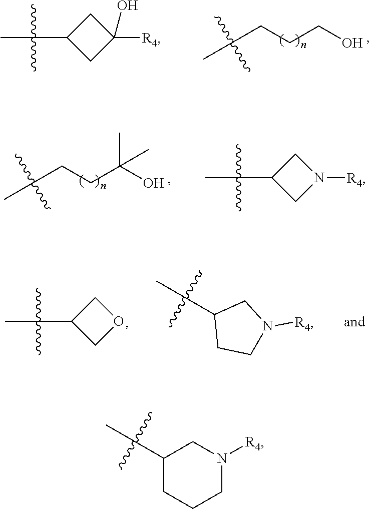 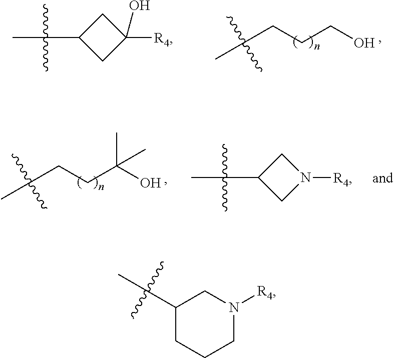
preferably
wherein R₄ is independently selected from —H or -C₁-C₃alkyl; and
n is selected from 0, 1 or 2.

10. The compound of formula I, I′, Ia, Ib, Ic, Id or Ie (preferably the compound of formula I′, Ib, Ic or Ie) according to claim 1,
wherein
R₀ is —H or —CH₃;
R₁ is —CF₃; and
R₂ is —OH.

11. The compound of formula I, I′, Ia, Ib, Ic, Id or Ie according to claim 1, which is selected from 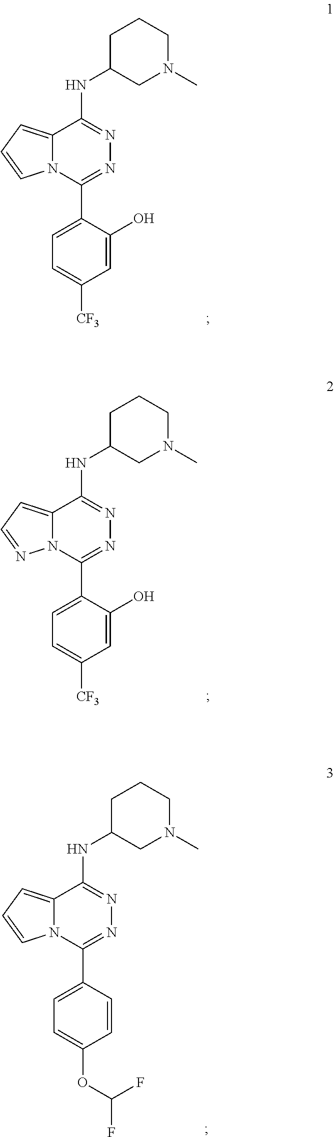 [SMILES: CN1CCCC(Nc2nnc(-c3ccc(C(F)(F)F)cc3O)n3cccc23)C1 ; CN1CCCC(Nc2nnc(-c3ccc(C(F)(F)F)cc3O)n3nccc23)C1 ; CN1CCCC(Nc2nnc(-c3ccc(OC(F)F)cc3)n3cccc23)C1] 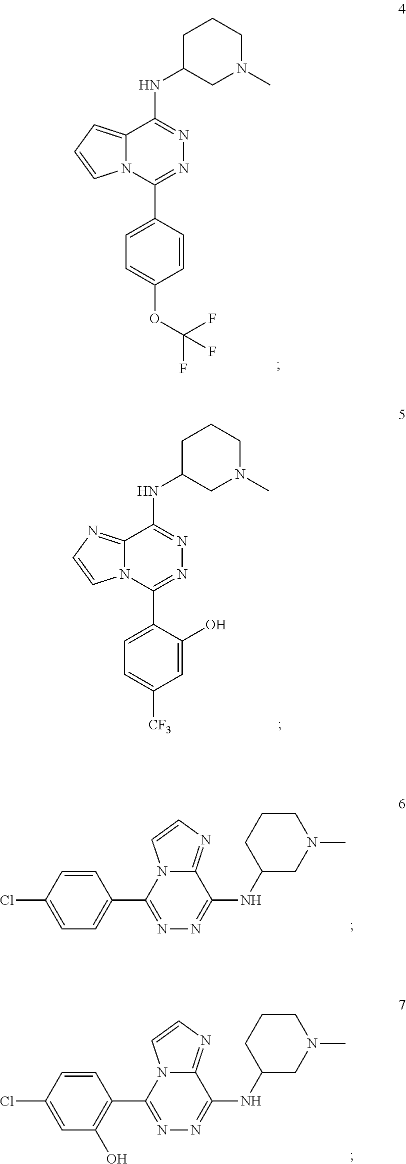 [SMILES: CN1CCCC(Nc2nnc(-c3ccc(OC(F)(F)F)cc3)n3cccc23)C1 ; CN1CCCC(Nc2nnc(-c3ccc(C(F)(F)F)cc3O)n3ccnc23)C1 ; CN1CCCC(Nc2nnc(-c3ccc(Cl)cc3)n3ccnc23)C1 ; CN1CCCC(Nc2nnc(-c3ccc(Cl)cc3O)n3ccnc23)C1] 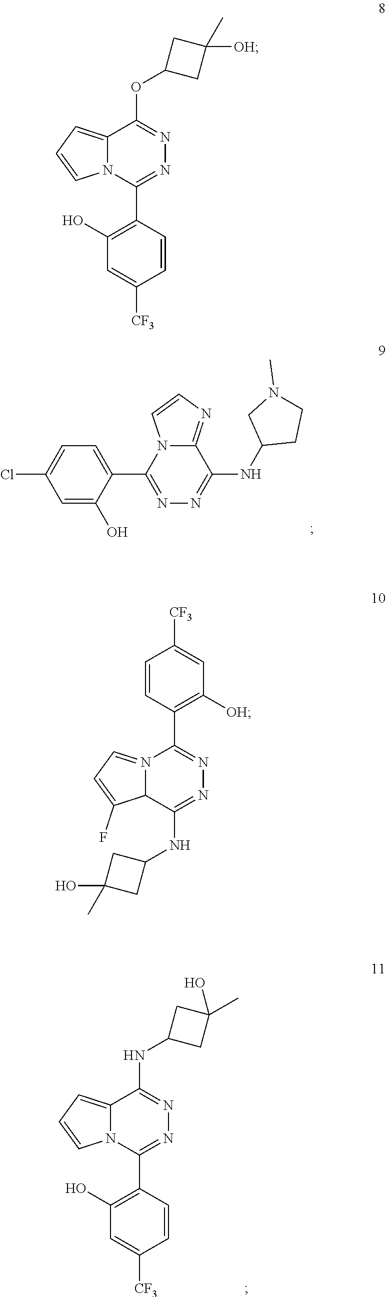 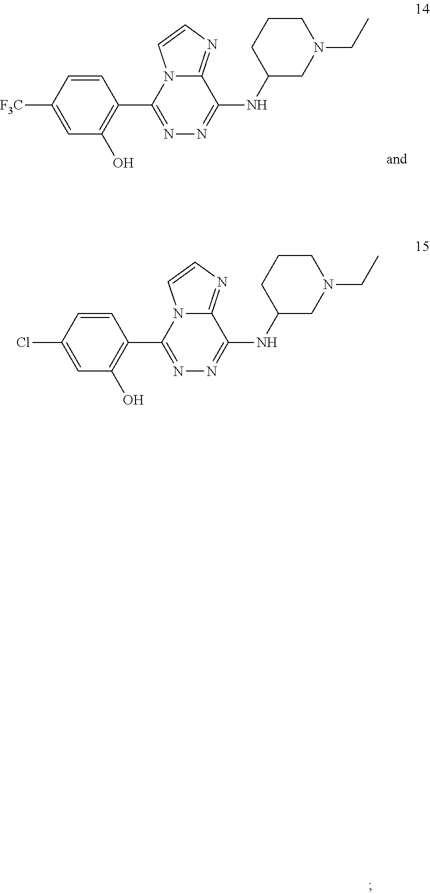 [SMILES: CCN1CCCC(Nc2nnc(-c3ccc(C(F)(F)F)cc3O)n3ccnc23)C1 ; CCN1CCCC(Nc2nnc(-c3ccc(Cl)cc3O)n3ccnc23)C1]
or a stereoisomer, a racemic mixture, a tautomer, a polymorph, a pharmaceutically acceptable salt, a prodrug, a hydrate, or a solvate thereof.

12. A pharmaceutical composition comprising a compound as defined in claim 1, or a stereoisomer, a racemic mixture, a tautomer, a polymorph, a pharmaceutically acceptable salt, a prodrug, a hydrate, or a solvate thereof, and optionally comprising at least one pharmaceutically acceptable carrier, diluent, adjuvant or excipient.

13. (canceled)

14. A method for the treatment, alleviation or prevention of a disease, or a disorder or an abnormality which is responsive to the modulation of a component of the NLRP3 inflammasome pathway and/or which is responsive to the modulation of IL-1 beta and/or IL-18 levels, comprising administering to a subject in need thereof an effective amount of the compound according to claim 1, or a stereoisomer, a racemic mixture, a tautomer, a polymorph, a pharmaceutically acceptable salt, a prodrug, a hydrate, or a solvate thereof.

15. The method according to claim 14, wherein the modulation is the reduction and/or inhibition of IL-1 beta.

16. The method according to claim 14, wherein the component of the inflammasome pathway is NLRP3 inflammasome.

17. The method according to claim 14 or 16, wherein the activation of NLRP3 inflammasome pathway is inhibited.

18. The method according to claim 14, wherein the disease, the disorder or the abnormality is selected from Alzheimer's disease, Parkinson's disease, amyotrophic lateral sclerosis, multiple sclerosis, demyelination, viral encephalitis, epilepsy, stroke, brain haemorrhage, atherosclerosis, asthma, allergic inflammation, cryopyrin-associated periodic syndromes (CAPS), Muckle-Wells syndrome (MWS), familial cold autoinflammatory syndrome (FCAS), neonatal-onset multisystem inflammatory disease (NOM ID), gout, pseudo-gout, inflammatory bowel disease, nonalcoholic fatty liver disease, nonalcoholic steatohepatitis, hypertension, myocardial infarction, oxalate-induced nephropathy, graft-versus host disease, type 1 diabetes, type 2 diabetes, rheumatoid arthritis, myelodysplastic syndrome, familial Mediterranean fever (FMF), TNF receptor associated periodic syndrome (TRAPS), mevalonate kinase deficiency (MKD), hyperimmunoglobulinemia D, periodic fever syndrome (HIDS), deficiency of interleukin 1 receptor (DIRA) antagonist, Majeed syndrome, acne, pyogenic arthritis pyoderma gangrenosum and acne (PAPA), haploinsufficiency of A20 (HA20), PLCG2-associated antibody deficiency and immune dysregulation (PLAID), pediatric granulomatous arthritis (PGA), PLCG2-associated autoinflammation, antibody deficiency and immune dysregulation (APLAID), sideroblastic anemia with B-cell immunodeficiency, periodic fevers, developmental delay (SIFD), chronic nonbacterial osteomyelitis (CNO), Sweet's syndrome, chronic recurrent multifocal osteomyelitis (CRMO), synovitis, pustulosis, acne, eczema, alopecia areata, actinic keratosis, hyperostosis, osteitis syndrome (SAPHO), multiple sclerosis (MS), psoriasis, Behcet's disease, Sjogren's syndrome, Schnitzler syndrome, chronic obstructive pulmonary disorder (COPD), steroid-resistant asthma, asbestosis, silicosis, cystic fibrosis, motor neuron disease, Huntington's disease, cerebral malaria, brain injury from pneumococcal meningitis, obesity, age-related macular degeneration (AMD), corneal infection, uveitis, dry eye, chronic kidney disease, diabetic nephropathy, alcoholic liver disease, skin contact hypersensitivity, sunburn, osteoarthritis, systemic juvenile idiopathic arthritis, adult-onset Still's disease, relapsing polychondritis, Chikungunya virus, Ross River virus, influenza, HIV, Coronaviruses, Dengue, Zika virus, hidradenitis suppurativa (HS), lung cancer metastasis, pancreatic cancers, gastric cancers, myelodisplastic syndrome, leukemia; polymyositis, colitis, helminth infection, bacterial infection, abdominal aortic aneurism, wound healing, depression, psychological stress, pericarditis including Dressler's syndrome, ischaemia reperfusion injury, frontotemporal dementia, HIV-associated neurocognitive disorder, Coronavirus-associated inflammatory pathologies, and traumatic brain and spinal cord injury, inflammatory pain, chronic pain, neuropathic pain, metastatic cancer-induced bone pain, chemotherapy induced peripheral neuropathy and migraine; preferably the disorder is selected from Alzheimer's disease, Parkinson's disease, amyotrophic lateral sclerosis, multiple sclerosis, demyelination, viral encephalitis, epilepsy, stroke, atherosclerosis, asthma, allergic inflammation, cryopyrin-associated periodic syndromes (CAPS), gout, inflammatory bowel disease, non-alcoholic fatty liver disease (NAFLD), non-alcoholic steatohepatitis (NASH), hypertension, myocardial infarction, oxalate-induced nephropathy, graft-versus host disease, type 1 diabetes, type 2 diabetes, rheumatoid arthritis, myelodysplastic syndrome, anti-neutrophil cytoplasmic antibody-associated vasculitis (AAV), lupus nephritis, anti-glomerular basement membrane (GMB) disease, IgA nephropathy, glomerulonephritis (GN), systemic lupus erythematosus (SLE), Focal Segmental Glomerulosclerosis, Minimal change disease (MCD), Psoriatic Arthritis, Hereditary Recurrent Fevers (HRFs), and amyloidosis (including AL amyloidosis, AA amyloidosis, ATTR amyloidosis, hereditary amyloidoses (including apolipoprotein A-I (AApoAI), apolipoprotein A-II (AApoAII), gelsolin (AGeI), fibrinogen (AFib), and lysozyme (ALys)), Beta-2 Microglobulin amyloidosis, iAPP amyloidosis).

19. The method according to claim 18, wherein the disease, the disorder or the abnormality is selected from Alzheimer's disease and Parkinson's disease.

20. The method according to claim 18, wherein the disease, the disorder or the abnormality is selected from cryopyrin-associated periodic syndromes (CAPS), non-alcoholic fatty liver disease (NAFLD), non-alcoholic steatohepatitis (NASH) and gout.

21. A method of analysis or in vitro screening, wherein the compound according to claim 1, or a stereoisomer, a racemic mixture, a tautomer, a polymorph, a pharmaceutically acceptable salt, a prodrug, a hydrate, or a solvate thereof, is applied as an analytical reference or an in vitro screening tool.
